(19) United States
(12) Patent Application Publication
(10) Pub. No.: US 2012/0171680 A1
(43) Pub. Date: Jul. 5, 2012

(54) SINGLE-MOLECULE PCR FOR AMPLIFICATION FROM A SINGLE NUCLEOTIDE STRAND

(76) Inventors: Ehud Y. Shapiro, Nataf (IL); Tuval Ben-Yehezkel, Ramat Gan (IL); Gregory Linshiz, Rehovot (IL); Shai Kaplan, Rehovot (IL); Uri Shabi, Kfar Saba (IL)

(21) Appl. No.: 12/997,601

(22) PCT Filed: Jun. 12, 2009
(86) PCT No.: PCT/IB2009/052508

§ 371 Date: Feb. 9, 2012

Related U.S. Application Data
(60) Provisional application No. 61/129,218, filed on Jun. 12, 2008.
(60) Provisional application No. 61/129,933, filed on Jul. 30, 2008.

(51) Int. Cl. C12Q 1/68; C12P 19/34

(52) U.S. Cl. 435/6.12; 435/91.2

(57) ABSTRACT

A method, apparatus and system for performing single molecule PCR for amplification from single stranded polynucleotides.

### FIELD OF THE INVENTION

The present invention is of a method, apparatus and system for performing single molecule PCR for amplification from a single strand polynucleotide.

### BACKGROUND OF THE INVENTION

The broad availability of synthetic DNA oligonucleotides has enabled the development of many powerful applications in biotechnology. Longer synthetic DNA molecules and libraries (made by the assembly of these oligonucleotides) in the 0.5-5 Kb range are now becoming increasingly available thanks to newly developed synthesis and error correction methods (1-7). Broad availability of such molecules, much needed since the advent of synthetic biology and modern genetic engineering, is expected to enable routine creation of new genetic material as well as offer an alternative to obtaining DNA from natural sources.

Unfortunately, the synthetic DNA oligonucleotides used as building blocks for making the longer constructs are error prone. Such errors accumulate linearly with the length of the constructed molecule and result in an exponential decrease in the fraction of error-free molecules. Hence an exponentially increasing number of molecules have to be screened, i.e. cloned into a host organism and sequenced, in order to obtain ever longer error-free molecules. In order to mitigate this effect a two-step assembly process (4, 7) is often used, in which fragments in the 500-1000 bp range are first screened via cloning and sequencing and then synthesis proceeds from the error-free clones.

In vivo cloning (1-7) is time consuming, manual-labor intensive, difficult to scale up and automate. This combined with the sheer number of clones that need to be screened to obtain long error-free synthetic DNA makes the cloning phase a bottleneck in de novo DNA synthesis and prevents synthetic DNA from being routinely produced in a fast, cheap and high-throughput manner. Reducing the number of clones required to obtain an error-free molecule is the subject of intensive ongoing research (1, 2, 4, 6), also recently addressed by the present inventors (5) with a method that relieves much of this burden.

However, there is another major issue for increasing the rapidity of DNA construction, namely replacing the time consuming and labor intensive in vivo cloning procedure associated with synthetic DNA synthesis with a faster and less laborious in vitro cloning procedure.

Since its introduction, PCR (8) has been implemented in a myriad of variations, one of which is PCR on a single DNA template molecule (9), which essentially creates a PCR “clone”. Single molecule PCR (smPCR) is a faster, cheaper, scalable, and automatable alternative to traditional in vivo cloning. Its standard application in molecular biology has been non-systematic, most commonly for the amplification of single molecules for sequencing, genotyping or downstream translation purposes (8-12). Recently, it has been systematically integrated into high-throughput DNA reading (sequencing) (13, 14).

### SUMMARY OF THE INVENTION

The background art does not teach or suggest a method, apparatus and system for performing single molecule PCR for amplification from single stranded polynucleotides. The background art also does not teach or suggest such a method, apparatus and system for constructing polynucleotides through the use of single molecule PCR (smPCR).

The present invention overcomes these drawbacks of the background art by providing, in at least some embodiments, a method, apparatus and system for performing single molecule PCR for amplification from single stranded polynucleotides. In some embodiments, the present invention also provides a method, apparatus and system for constructing polynucleotides, optionally and preferably as a process for in vitro cloning, for example, as well as for other types of polynucleotide synthesis procedures, including without limitation the widely used two step assembly PCR method (7).

According to some embodiments of the present invention, the method, apparatus and system for polynucleotide construction preferably also incorporates the recursive synthesis and error correction procedure of the present inventors, known as the “Divide and Conquer” (D&C) method, with smPCR. The D&C method (5), which combines recursive synthesis and error-correction, operates as follows. D&C is used in silico to divide the target DNA sequence to be constructed into fragments short enough to be synthesized by conventional oligo synthesis, albeit with errors (15); these oligos are synthesized and are recursively combined in vitro, forming target DNA molecules with roughly the same error rate as the source oligos; error-free parts of these molecules, identified by cloning and sequencing, are extracted and used as new, typically longer and more accurate inputs to another iteration of the recursive synthesis procedure. Typically, an error-free clone is obtained after one iteration of this procedure.

According to other embodiments, the present invention provides a method, system and apparatus for bar coding molecules for polynucleotide construction.

According to still other embodiments, the present invention provides use of Real-Time PCR for determining the dilution required for single molecule amplification.

As defined herein, the term “in vivo” relates to the environment of living matter, such as a cell for example. For example, cloning performed in bacteria, yeast, mammalian cell lines or indeed any type of cell is referred to herein as “in vivo cloning”. The term “in vitro” relates to an environment free of any living matter, although potentially including proteins, nucleotides and so forth, as described in greater detail below.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

Where ranges are given, endpoints are included within the range. Furthermore, it is to be understood that unless otherwise indicated or otherwise evident from the context and understanding of one of ordinary skill in the art, values that are expressed as a range can assume any specific value or subrange within the stated range in different embodiments of the invention, to the tenth of the unit of the lower limit of the range, unless the context clearly dictates otherwise. Where a percentage is recited in reference to a value that intrinsically has units that are whole numbers, any resulting fraction may be rounded to the nearest whole number.

In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A and 1B describe an exemplary method for performing the smPCR process according to some embodiments of the present invention in place of in vivo cloning;

FIG. 2 relates to the problem of primer dimers and anticipation;

FIG. 3A shows the percent of molecules that are error-free as a function of construct length for the typical range of error-rate of synthetic oligos;

FIG. 3B shows the number of clones required in order to obtain error-free synthetic molecules using different construction methods as a function of construct length;

FIG. 3C shows the percent of dsDNA that is homoduplex as a function of DNA length;

FIG. 4 shows the effect of termination time on the formation of homodimers;

FIG. 5 shows that hetero-dimers hinder smPCR;

FIG. 6 shows the effect of dilution on PCR;

FIG. 7 shows that the number of cycles required for single molecule amplification can be accurately anticipated given the initial and final amount of DNA in a PCR with known amplification efficiency, as for the above described experimental efficiency;

FIG. 8 shows the use of randomized primers and the results thereof;

FIG. 9 shows that the population of molecules featuring such an error is reduced as the cycle number increases during which the error is inserted;

FIG. 10 shows the average error-rate of DNA molecules amplified from a single error-free molecule using PCR with Taq polymerase as a function of number of PCR cycles performed;

FIG. 11 relates to the average error-rate of DNA molecules amplified from a single error-free molecule using PCR with Taq polymerase as a function of number of PCR cycles performed;

FIG. 12 shows the results of experiments with a proof reading polymerase, indicating that error-free molecules are readily cloned using smPCR;

FIG. 13 shows an overview of the process for constructing a 1.8 Kb polynucleotide using the smPCR procedure; and

FIGS. 14-16 show the results of an exemplary construction process according to some embodiments of the present invention.

### DETAILED DESCRIPTION OF SOME EMBODIMENTS

The present invention provides, in at least some embodiments, a method, apparatus and system for performing single molecule PCR for amplification from single stranded polynucleotides. In some embodiments, the present invention also provides a method, apparatus and system for constructing polynucleotides, optionally and preferably as a process for in vitro cloning, for example, as well as for other types of polynucleotide synthesis procedures, including without limitation the two step assembly PCR method.

According to some embodiments of the present invention, the method is combined with the D&C method for construction with error correction.

### EXAMPLES SECTION

This Section relates to some illustrative, non-limiting Examples for implementing various embodiments of the present invention.

### Example 1

### smPCR for In Vitro Cloning

This non-limiting, illustrative Example shows that in vitro cloning based on smPCR can be used as a practical alternative to conventional in vivo cloning by using the below described, illustrative, DNA synthesis protocol. In particular, a 1.8 Kb-long DNA molecule was successfully constructed from synthetic unpurified oligos using the recursive synthesis and error correction procedure of the present inventors with smPCR, and as a control also constructed the same molecule using conventional in vivo cloning. The results are compared below.

The throughput of DNA reading (sequencing) has dramatically increased recently due to the incorporation of in vitro clonal amplification. The throughput of DNA writing (synthesis) is trailing behind, with cloning and sequencing constituting the main bottleneck. To overcome this bottleneck, an in vitro alternative for in vivo DNA cloning must be integrated into DNA synthesis methods. This Example shows how a new smPCR-based procedure can be employed as a general substitute to in vivo cloning thereby allowing for the first time in vitro DNA synthesis. Although this Example demonstrates incorporating smPCR in a particular method, the approach is general and can be used in principle in conjunction with other DNA synthesis methods as well.

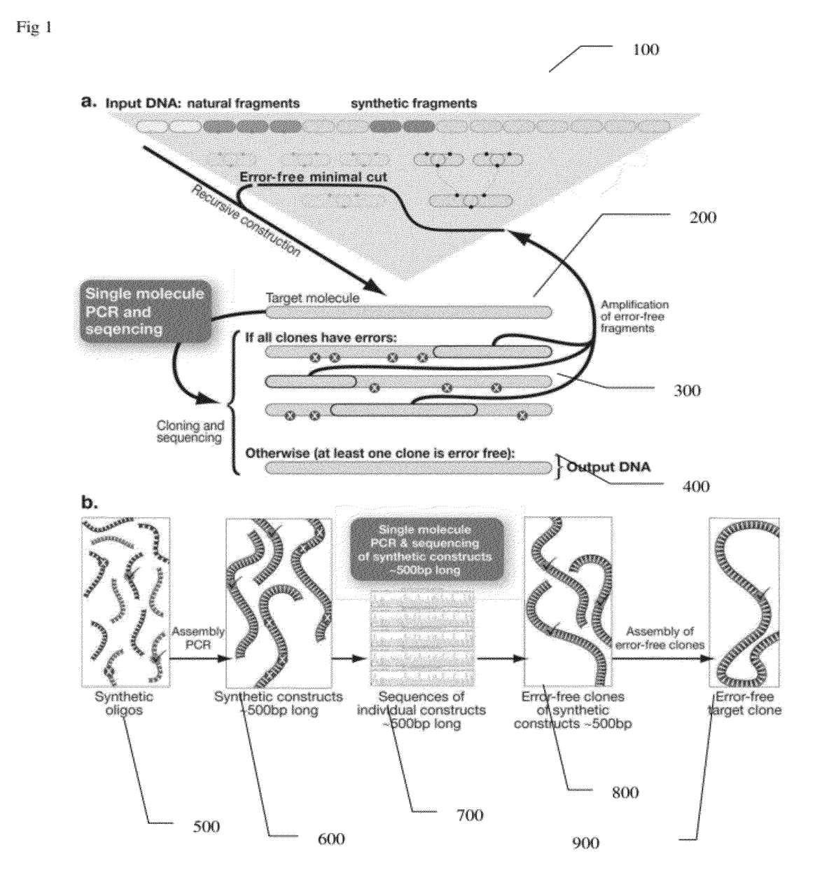

The overall method is described with regard to FIGS. 1A and 1B, which describe an exemplary method for performing the smPCR process according to some embodiments of the present invention in place of in vivo cloning.

FIG. 1A shows that target synthetic molecules are recursively constructed from oligos and then error-corrected using the new smPCR procedure instead of in vivo cloning. In brief, in stage 100, preparation of the target DNA molecules (which as shown may optionally be natural and/or synthetic fragments) for smPCR amplification is carried out by a PCR process that introduces sites for the smPCR primer. This PCR process is preferably stopped at the exponential phase of amplification so that heterodimers are not formed. The PCR products are then diluted according to calculations and experimental results and used as template for smPCR with a special primer (in this example and for the purposes of illustration only, a C-A primer) that doesn't produce non-specific amplification products, as shown in stage 300. The DNA “clones” amplified using smPCR are then sequenced and an error-correction process is performed, in stage 300, using the smPCR amplified molecules as starting material until an error free molecule is obtained, as shown in stage 400.

FIG. 1B shows a conceptual illustration of how the smPCR procedure could also be used in principle, with a two-step assembly PCR. From left to right, in box 500, oligos are assembled in groups and amplified to yield fragments 400-500 bp long, as shown in box 600. These could be cloned using exactly the same smPCR procedure described in this work and sequenced, as shown in box 700. The error-free clones are then selected for further assembly of the target sequence using various methodologies, as shown in box 800, to produce a final error-free target clone 900.

Optionally the process may be automated with the use of a robot for example, in which the initial material is placed in a container. As described in greater detail below, the oligonucleotides and/or polynucleotides are labeled, for example with the bar code method described below. The container is then optionally placed within a PCR machine (or alternatively the container is stationary and the PCR machine is moved) for performing the necessary PCR reactions. The robot then preferably dilutes the solution to a single molecule dilution, as described in greater detail below, after which the container is again located within the PCR machine. This process is optionally repeated one or more times.

The results of this process may optionally then be examined with sequencing and/or subjected to one or more other procedures, including but not limited to cleaning and purification, cloning, enzymatic reaction or any other process for which polynucleotides may optionally be used.

The process may optionally be completely automated in terms of production of the polynucleotide, thereby enabling cloning to be performed automatically, in vitro, without the requirement for whole cells or any cellular material apart from the enzymes etc required for performing PCR, such that the process is not performed within any living matter. Thus, there are no problems of biohazards, requirements for manually performed processes and so forth.

As noted above the smPCR process according to the present invention is performed with single stranded polynucleotides, which has many advantages. Without wishing to be limited, use of single stranded polynucleotides enables the process to be performed completely in vitro, thereby avoiding the problems associated with in vivo cloning (ie cloning within a living cell). Also the use of such polynucleotides enables a homogenous population of molecules to be amplified and avoids the problems associated with heterodimer formation, also as described in greater detail below.

Specific description of more detailed exemplary, illustrative methods is provided below, with regard to a particular non-limiting experimental example. Some of the general methods used herein are described as non-limiting examples before the more detailed description of the exemplary materials and methods.

Description of the Recursive Construction Method

Divide and Conquer, the quintessential recursive problem solving technique, was applied to divide the target DNA sequence in silico into fragments short enough to be synthesized by conventional oligo synthesis, albeit with errors due to the oligos; these error-prone molecules are recursively combined in vitro, forming error-prone target DNA molecules; error-free parts of these molecules are identified, extracted and used as new, typically longer and more accurate, inputs to another iteration of the recursive construction procedure. One execution of this procedure typically yields error free molecule. Nevertheless, in principle, if errors remain the entire process can be repeated until an error-free target molecule is formed.

Description of the Error Correction Method

In general, a composite object constructed from error-prone building blocks is expected to have a higher number of errors than each of its building blocks. However, if errors are randomly distributed among the building blocks and occur randomly during construction, and if several copies of an object are constructed, it is expected that few if not all of the error prone copies would contain some error-free components with a certain minimal size. Moreover, based on the known rate and distribution of errors it is possible to predict a specific property of these error-free components, namely the number of times they will occur in a given number of constructed objects. Furthermore, it is possible to calculate the probability that a certain number of error-free components would collectively span the entire target object.

Conversely (and more importantly), it is possible to calculate the number of object copies (clones) required so that their error-free components span the entire target object with a desired probability. If such components could be identified and utilized from the faulty objects, they could be reused as building blocks for another recursive construction of the object.

Based on this observation, the recursive construction procedure may optionally be re-applied to correct errors in synthetic constructed molecules, as follows: error-free parts of the erroneous target DNA molecules are identified by cloning and sequencing and used as new, typically longer, inputs to the same recursive construction procedure. Since this construction starts from typically larger DNA building blocks that are error-free, the number of errors in the resulting reconstructed DNA is expected to decrease, possibly down to zero, eschewing additional screening of clones.

Description of the Minimal Cut

A cut in a tree is a set of nodes that includes a single node on any path from the root to a leaf. Let T be a recursive construction protocol tree and S a set of strings. We say that S covers T if there is a set of strings C such that every string in C is a substring of some string in S and C is a cut C of T. In such a case we also say that S covers T with C.

Claim: If S covers T, then there is a unique minimal set C such that S covers T with C. Proof: Given an RC protocol T and a set of subcomponents S, find a minimal C such that S covers T with C. Then C is created and the recursive construction is performed starting with C.

Computing the Minimal Cut

A recursive approach is used for computing the minimal cut of a protocol tree. Each node in the tree represents a biochemical process with a product and two precursors. The algorithm starts with the root of the tree (target molecule) and for each node checks whether its product sequence exists with no errors in one of the clones. If such a clone exists this product is marked as a new basic building block for reconstruction of the target molecule and its primer pair and relevant clone (as template) are registered as its generating PCR reaction. If there is no clone which contains an error free sequence of the node product the reaction is registered as existing reaction in the new protocol and the algorithm is recursively executed on the two precursors of the product. The output of such a protocol is a tree of reactions which comprises a minimal cut of the original tree. It contains leaves for which error free products exist and that all its internal nodes are have no error free clone that contain them. An automated program that utilizes these new error free building blocks for recursive construction of the target molecule is generated for the robot.

Materials & Methods

RT-PCR (Real Time PCR)

All PCRs were performed using the Bio-Rad MyiQ Single-Color Real-Time PCR Detection System.

Capillary Electrophoresis Fragment Analysis

Fragment analysis of PCR products was performed to single base pair resolution using an ABI analyzer and the LIZ500(−250) size marker (see below for a detailed description).

Cloning

Fragments were cloned into the pGEM T easy Vector System 1 from PROMEGA. Vectors containing cloned fragments were transformed into JM109 competent cells from PROMEGA1 and sequenced.

Single Molecule PCR

smPCR was performed with hot-start Accusure (BioLine) for the longer Mitochondrial and with Taq Polymerase (ABgene) for the GFP fragment:

Template concentration was determined according to calculations described in the paper and dissolved in 5 ul DDW. 10 pmol of the CA primer dissolved in 10 μl DDW. Reaction contained 25 mM TAPS pH 9.3 at 25° C., 2 mM MgCl₂, 50 mM KCl, 1 mM β-mercaptoethanol, 200 μM each of dNTP, 1.9 units AccuSure DNA Polymerase (BioLINE).

RT-PCR Thermal Cycler program: Enzyme activation at 95° C. 10 min, Denaturation 95° C. 30 sec, Annealing at Tm of primers 30 sec, Extention 72° C. 1.5 min per Kb, 50 cycles. It is important that the PCR is prepared in a sterile environment using sterile equipment and uncontaminated reagents.

Description of the Calibration Experiment for Correctly Determining the Required Dilution Factor to Reach the Optimal Concentration.

For this, RT-PCR amplification of the synthetic construct to be cloned was terminated within the phase of exponential amplification (see below for a description). The terminated PCR was then diluted to a few different concentrations and pools of 96 PCR's were performed using each dilution as template. The ratio of amplified vs. non-amplified reactions was determined for each dilution pool. The dilution which resulted in the correct amplification ratio (i.e. close to the calculated optimal concentration of template specified in supplementary methods) was chosen as the required dilution factor for PCR's from then on. An important but non-limiting factor is that the RT-PCR preceding the smPCR is optimally terminated at a specific stage of the amplification process, as determined by the RT-PCR curve (see below for a description). After this calibration, accurate dilutions for smPCR were made easy by terminating the PCR preceding the smPCR at the predetermined stage and making the predetermined dilution.

Chemical Oligonucleotide Synthesis

Oligonucleotides for all experiments were ordered by commercial providers (Sigma Genosys & IDT) with standard desalting.

DNA Purification

Manual DNA Purification was performed with QIAGEN's MinElute PCR purification kit using standard procedures.

Methods for Recursive Construction and Error Correction.

The core recursive construction and reconstruction (error-correction) step requires four basic enzymatic reactions: phosphorylation, elongation, PCR and Lambda exonucleation. They are described in the order of execution by the protocol of the present inventors.

Phosphorylation of all PCR primers used by the recursive construction protocol is performed beforehand simultaneously, according to the following protocol:

300 μmol of 5′ DNA termini in a 50 μl reaction containing 70 mM Tris-HCl, 10 mM MgCl₂, 7 mM dithiothreitol, pH 7.6 at 37° C., 1 mM ATP, 10 units T4 Polynucleotide Kinase (NEB). Incubation is at 37° C. for 30 min, inactivation 65° C. for 20 min.

Overlap Extension Elongation Between Two ssDNA Fragments:

1-5 pmol of 5′ DNA termini of each progenitor in a reaction containing 25 mM TAPS pH 9.3 at 25° C., 2 mM MgCl₂, 50 mM KCl, 1 mM β-mercaptoethanol 200 μM each of dNTP, 4 units Thermo-Start DNA Polymerase (ABgene). Thermal cycling program is as follows: Enzyme activation at 95° C. 15 min, slow annealing 0.1° C./sec from 95° C. to 62° C., elongation at 72° C. for 10 mM.

PCR Amplification of the Above Elongation Product with Two Primers, One Of which is Phosphorylated:

1-0.1 fmol template, 10 pmol of each primer in a 25 μl reaction containing 25 mM TAPS pH 9.3 at 25° C., 2 mM MgCl₂, 50 mM KCl, 1 mM β-mercaptoethanol 200 μM each of dNTP, 1.9 units AccuSure DNA Polymerase (BioLINE). Thermal Cycler program is: Enzyme activation at 95° C. 10 min, Denaturation 95° C., Annealing at Tm of primers, Extention 72° C. 1.5 min per kb to be amplified 20 cycles.

Lambda Exonuclease Digestion of the Above PCR Product to Re-Generate ssDNA:

1-5 pmol of 5′ phosphorylated DNA termini in a reaction containing 25 mM TAPS pH 9.3 at 25° C., 2 mM MgCl₂, 50 mM KCl, 1 mM β-mercaptoethanol 5 mM 1,4-Dithiothreitol, 5 units Lambda Exonuclease (Epicentre). Thermal Cycler program is 37° C. 15 min, 42° C. 2 min, Enzyme inactivation at 70° C. 10 min.

Results

An error free 1.8 Kb molecule was constructed from synthetic unpurified oligos using recursive synthesis and error correction with in vitro cloning based on smPCR. At the same time the exact same procedure was performed but with traditional in vivo cloning as a control. The results show that the smPCR-based procedure is comparable to traditional cloning in terms of the fidelity of the clones. Although the accuracy of in vivo cloning is higher than smPCR, this has a minor effect on the number of clones required to obtain an error-free clone for molecules in the several-Kb range. The relatively small difference in fidelity is greatly outweighed by the improved time, cost and throughput offered by the in vitro procedure.

Preferably several modifications are incorporated into smPCR methodology according to at least some embodiments of the present invention in order for it to be suitable for de novo DNA synthesis, as discussed in the results section below. These included improved primer selection, computational optimization and experimental calibration of template concentration, real-time diagnosis of faulty reactions, avoiding the cloning of heteroduplexes, bar-coding molecules and creating a process with adequate fidelity.

Careful Selection of Adequate Primers is Needed to Enable Single Molecule Amplification

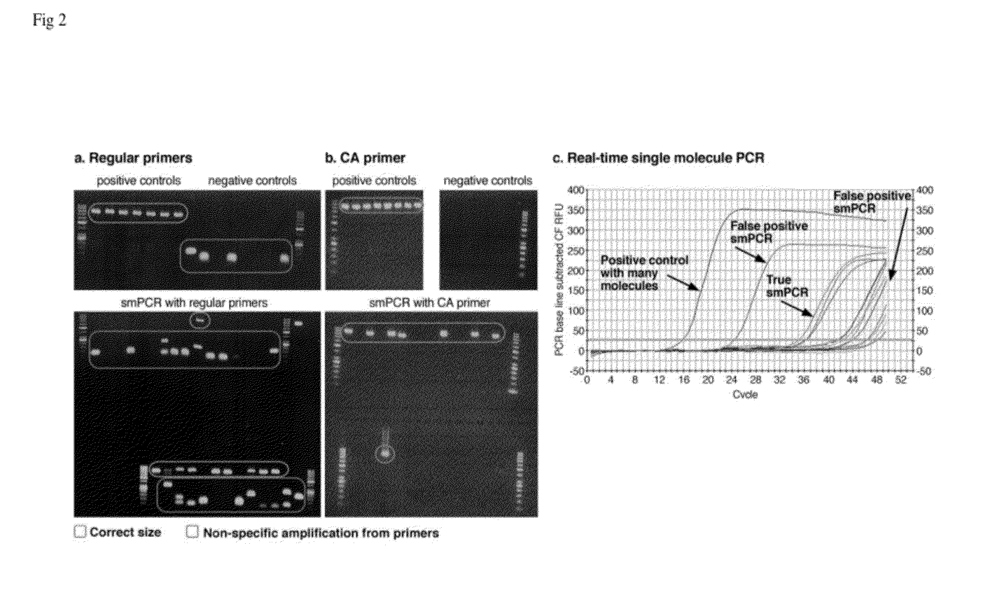

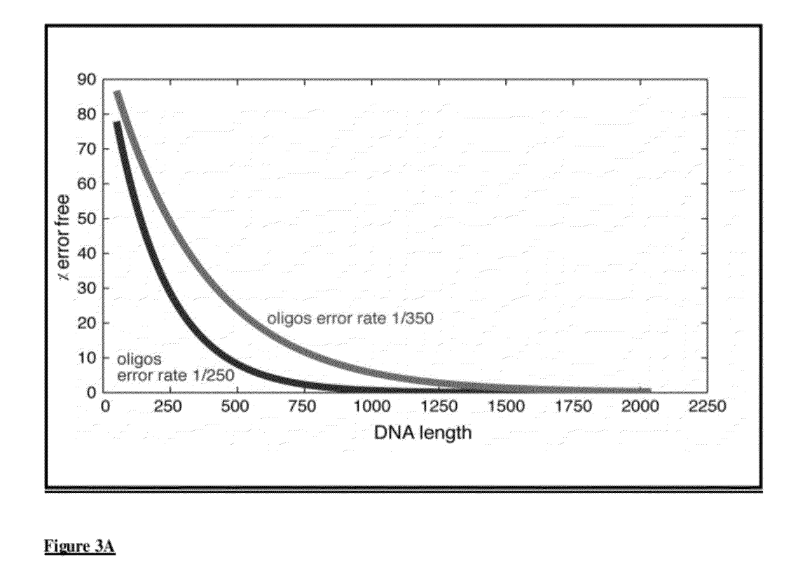

smPCR amplification requires extensive cycling (9-12). This often leads to the amplification of non-specific products originating from interaction between the PCR primers, as shown with regard to FIG. 2A. This often inhibits the amplification of the single molecule template, typically resulting in either no amplification of the target molecule due to dimer formation or in amplification of the primer dimer on top of the correct PCR product. Consequently, a large fraction of the smPCRs performed cannot be used for synthesis since they didn't amplify or have non specific amplification products. This has to be compensated for by performing more smPCRs than are actually needed for synthesis.

FIG. 2 relates to the problem of primer dimers and anticipation. Adequate selection of primers leads to improved specificity in smPCR; RT-PCR can distinguish true single-molecule PCRs from false positives. As part of this embodiment of the present invention, a special primer was designed for smPCR as described below.

FIG. 2A shows smPCRs with regular primers show many non-specific amplification products. Top gel: Lanes 1-7: positive control (many template molecules) PCRs show bands at the correct size. Lanes 8-15: no-template control PCRs have non-specific amplification from primers. Bottom gel: smPCR experiments—a large fraction of reactions show non-specific amplification from primers which inhibit smPCR and hinder its use.

To solve this problem a special primer was designed for smPCR consisting of a single sequence (complementary to both ends of the single molecule template) which contains a sequence of Cytosine and Adenine DNA bases only, referred to herein as the “C-A primer” or “CA primer”. It was thought that this should reduce the formation of PCR products that originate from primer-primer interactions due to the non-complementary nature of the Cytosine and Adenine bases. This successfully eliminated non-specific amplification resulting from interaction between primers and its inhibiting effect on single molecule amplification, which in turn significantly decreased the total number of PCRs needed to obtain the minimal number of smPCR clones required for synthesis of error-free DNA. The sites for the C-A primer (as well as the random bar coding bases to be discussed later on) at the termini of the target molecules are incorporated by either an a-priori PCR or during the synthesis of the molecule as part of the target sequence.

FIG. 2B shows that smPCRs with the CA primer provide specific amplification. Top left gel: positive control (multiple template molecules) PCRs show bands at the correct size. Top right gel: no-template control PCRs do not have non-specific amplification. Bottom gel: smPCR experiments bands at the correct size and frequency with no non-specific amplification

FIG. 2C shows that real-time PCR helps determining whether PCRs are true single-molecule PCRs or false positives due to non-specific amplification from primers or contamination.

Heteroduplexes Prevent In Vitro Cloning of Synthetic DNA

Initially, the sequencing of all true smPCR experiments resulted in shifted sequencing chromatograms which could not be read properly, despite the fact that in vivo clones from the same DNA sequenced correctly. The cause of this turned out to be that de novo constructed DNA is double stranded (1-4, 6, 7), with each strand having different errors originating from different synthetic oligo species. Performing smPCR on such a heteroduplex creates two distinct populations of amplified molecules, one from each strand. The abundance of deletions and insertions in synthetic oligos (4, 15) causes the sequencing chromatograms of these dual population PCRs to be frame shifted and their sequence cannot be determined.

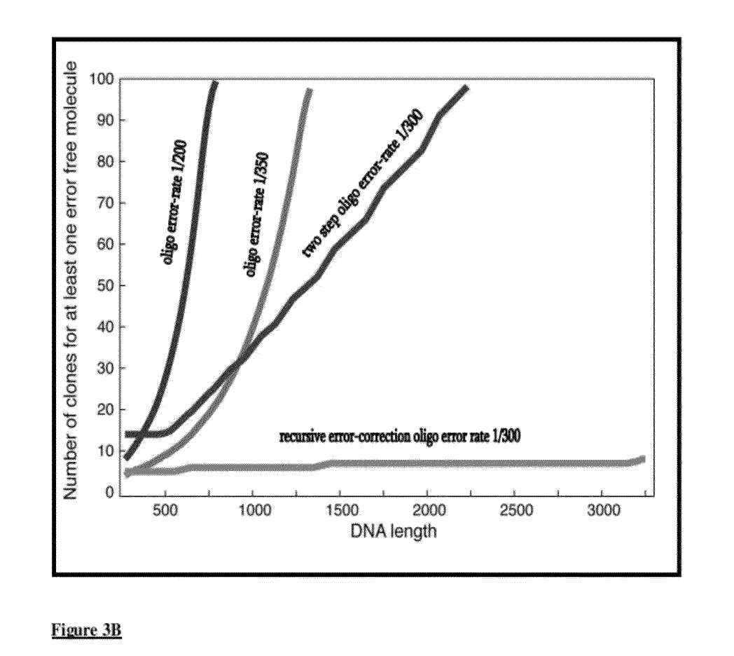

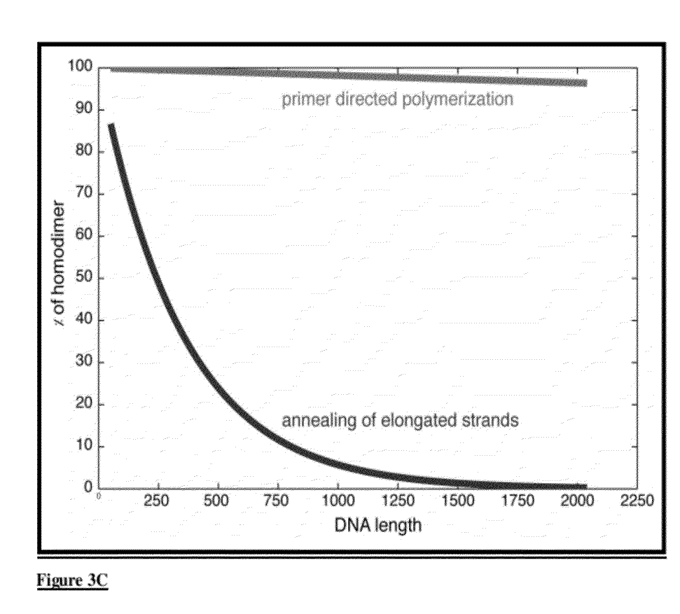

FIG. 3A shows the percent of molecules that are error-free as a function of construct length for the typical range of error-rate of synthetic oligos (and hence of constructs). The right curve shows an error-rate of 1/350 and is labeled “oligos error rate 1/350”; the left curve shows an error rate of 1/250 and is labeled “oligos error rate 1/250”. The high error-rate results in a large drop in the fraction of error-free molecules even in short fragments 500-1000 bp long.

FIG. 3B shows the number of clones required in order to obtain error-free synthetic molecules using different construction methods as a function of construct length. The error rates are as follows: green plot—error-rate 1/350. blue plot 1/200. red plot 1/300 two step construction. cyan plot 1/300 using recursive construction and error-correction. Here all construction methods are assumed.

These smPCR cloning results were reinforced by calculations that show that, according to the error-rate of oligos (4, 15), heteroduplexes are much more abundant than homoduplexes at the typical cloning length, as demonstrated by FIG. 3C. FIG. 3C shows the percent of dsDNA that is homoduplex as a function of DNA length. The lower plot, labeled “annealing of elongated strands”, shows PCR that is allowed to cycle past the phase of 100% amplification efficiency. The upper plot, labeled “primer directed polymerization”, shows PCR that is not allowed to cycle past the phase of 100% amplification efficiency. The y-axis shows the percent of homodimers formed, while the x-axis shows the length of the DNA formed during PCR. In practice almost all synthetic clones were heteroduplexes (due to insertions or deletions) which could not be sequenced properly.

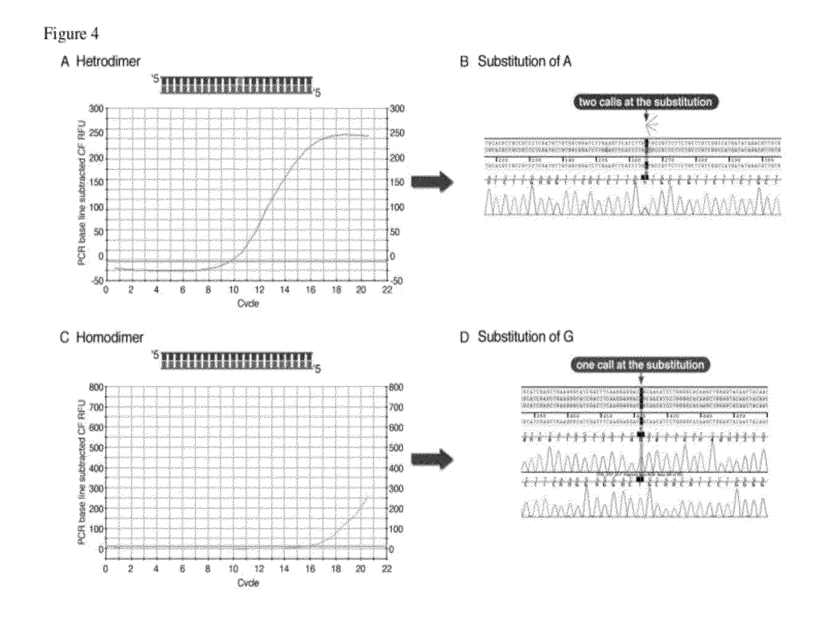

Rare exceptions were clones that were heteroduplexes only due to substitutions in one or both strands (which do not result in frame-shifts) and were therefore sequenced properly. These results are shown in FIG. 4A, which shows that over-cycling of the PCR past the phase of 100% amplification efficiency leads to the formation of hetero-dimers. FIG. 4B shows that the sequencing chromatogram of a PCR amplified substitution hetero-dimer shows 2 different base calls at the mutation but are not frame-shifted from the site of the mutation. In this diagram, substitution is with the nucleotide “A”.

FIG. 4C shows that a PCR process that is terminated before the end of 100% amplification efficiency generates homodimers, not hetero-dimers. FIG. 4D shows that the sequencing chromatograms of homodimers are readable and not frame-shifted and always show a single base call at each base even if one or more mutations (with respect to the target sequence) are present. In this diagram, substitution is with the nucleotide “G”.

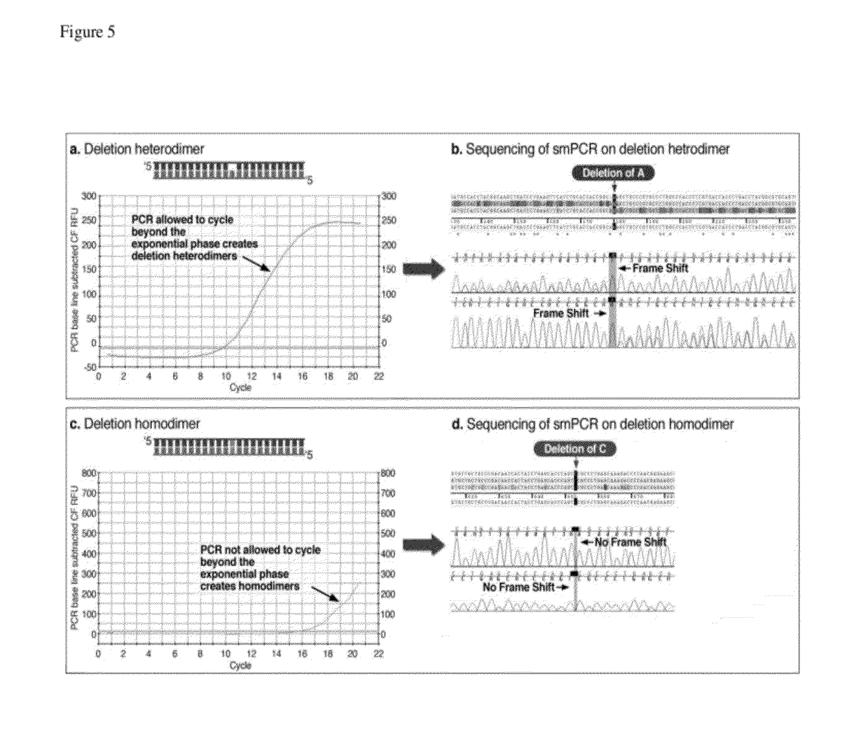

FIG. 5 shows that hetero-dimers hinder smPCR. The template for smPCR is produced with an ordinary PCR reaction. If this PCR is not terminated at the exponential phase of amplification it produces heterodimers, which hinder smPCR. FIG. 5A shows that over-cycling of the PCR past the exponential phase of amplification leads to the formation of hetero-dimers by re-annealing of different elongated strands; the inflection point is indicated with an arrow. The y-axis is the PCR base line; the x-axis refers to the number of cycles. The graphic above the plot shows a schematic heterodimer.

FIG. 5B shows that the sequencing chromatograms of both sense and anti-sense strands of a PCR amplified hetero-dimer are frame-shifted and unreadable from the site of the (insertion or deletion) mutation and on. In this case, the insertion is of the nucleotide “A”, thereby causing a frame shift.

FIG. 5C shows that a PCR terminated before the end of the exponential amplification generates homodimers, not hetero-dimers (x-axis and y-axis are as for FIG. 5A). The graphic above the plot shows a schematic homodimer.

FIG. 5D shows that the sequencing chromatogram of a PCR amplified homodimer is readable and not frame-shifted even if one or more mutations (with respect to the target sequence) are present. For example, deletion of the nucleotide “C” as shown does not result in frame shifting.

The reason that heteroduplexes were not reported to be a problem so far in de novo synthesis (1-4, 6, 7) is probably the ubiquitous use of in vivo cloning, which converts the erroneous mismatched DNA into perfectly matched DNA, albeit erroneous compared to the target sequence. A true smPCR should therefore be performed on either one ssDNA molecule or on two perfectly complemented molecules, i.e. one homoduplex dsDNA.

As suggested by the above results, according to some embodiments of the present invention, generating homoduplex dsDNA may be performed by terminating the PCR amplification of synthetic DNA prematurely, not allowing it past the exponential phase of amplification, as monitored by RT-PCR and as shown above. Terminating the PCR at the exponential phase of amplification assures that each dsDNA molecule is formed by primer-directed polymerization which forms homoduplexes, and not by the annealing of previously elongated strands which forms heteroduplexes. A comparison between smPCRs executed using templates generated by primer-directed polymerization and by annealing of previously elongated strands are shown above.

According to alternative embodiments of the present invention, although optionally this method may be used in addition to the above, synthetic dsDNA constructs labeled with a 5′ phosphate at one end were treated with Lambda exonuclease to convert them into ssDNA. smPCR on ssDNA templates generated by this enzymatic treatment indeed resulted in a larger fraction of smPCRs which can be sequenced.

Computational Optimization and Experimental Calibration of Template DNA Concentration

smPCR reactions are generally similar to regular PCR reactions in their basic biochemistry, the difference is that while PCR typically start the amplification with multiple copies of the template molecule, the goal in smPCR is to amplify a single template molecule. This is achieved by diluting a solution with template molecules in a known concentration so that the template aliquot is expected to have about one molecule. As the dilution is a stochastic process, at any such dilution some aliquots would have no template molecule and some would have multiple template molecules. As these two cases cannot be avoided, smPCR is done as a batch of multiple parallel reactions, with the hope that at least some would be true smPCRs, namely successful PCR reactions that amplify single template molecules. “False positive” smPCR's, which amplify multiple template molecules, are identified using sequencing as described in the previous example. The cost of sequencing is a major component of synthetic DNA synthesis, and the sequencing of false positives can render smPCR unpractical if their fraction in the total number of reactions is too high.

Standard gel/capillary electrophoreses (C.E)/real-time PCR(RT-PCR) analyses can be used to differentiate no-template (negative) reactions from (positive) PCRs with template, however, they cannot be used to differentiate a true smPCR from false positive reactions.

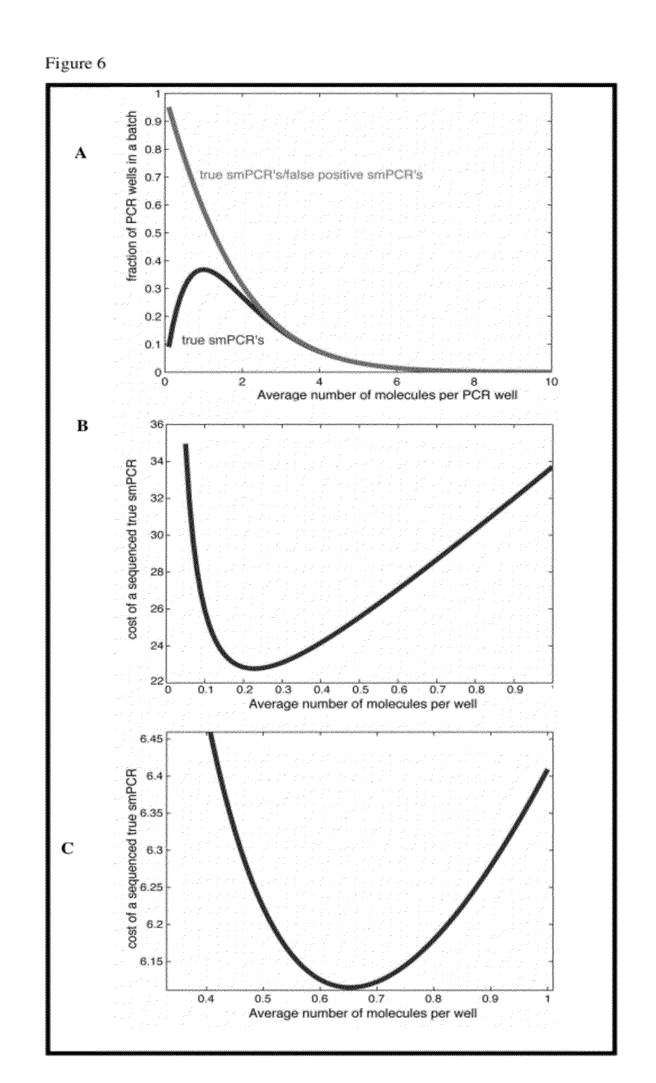

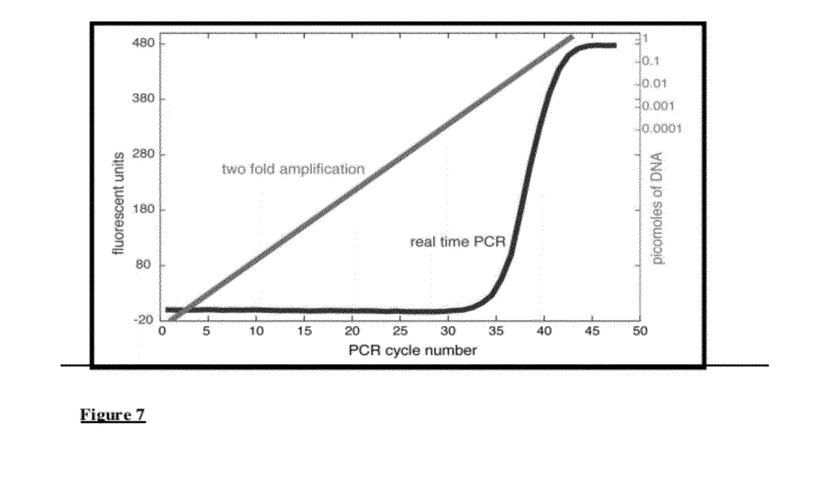

FIG. 6A shows the average number of molecules per PCR well Vs. fraction of reactions. The lower plot, labeled “true smPCRs”, shows PCR's that have exactly 1 molecule out of all the PCR's performed. The upper plot, labeled “true smPCRs/false positive smPCRs”, shows reactions that have exactly 1 molecule out of all the reactions that amplified (i.e. excluding those with zero molecules that didn't amplify). The x-axis shows the average number of molecules per well, while the y-axis shows the fraction of wells in a batch.

As shown, diluting the template to one molecule per well on average maximizes the fraction of true smPCRs out of all the reactions in the batch (FIG. 6A, lower curve). However, it does not maximize the ratio of true smPCRs to false positives (FIG. 6A, upper curve) which is important for avoiding futile sequencing. For example, aiming for one molecule per well on average leads to >50% futile sequencing of false positives (FIG. 6A, lower curve). Further reducing template concentration reduces the extent of futile sequencing of PCRs with multiple template molecules, however, it increases the extent of futile PCRs due to no-template reactions.

Determining the template concentration that would result in an optimal ratio between true smPCRs, false positives and no-template reactions can only be determined by associating a cost to performing sequencing and smPCR reactions. FIGS. 6B and C show the average number of molecules per PCR well vs. cost of obtaining a sequenced true smPCR. FIG. 6B shows that the cost of sequencing is 12 times higher than PCR. The x-axis shows the average number of molecules per well, while the y-axis shows the cost of a sequenced true smPCR. FIG. 6C shows that sequencing and PCR have equal cost (axes are identical to those of FIG. 6B). Higher Sequencing/PCR cost ratios shift the minimum of the graph (minimal cost for obtaining a sequenced smPCR) to fewer molecules per well and vice versa.

The optimal concentration to be ˜0.6 template molecules per smPCR well if an equal cost is associated with smPCR and sequencing and ˜0.2 molecules per well if sequencing is assigned the more realistic cost of 8 times that of smPCR. Performing smPCRs at the optimal template concentration reduces the overall cost of obtaining each sequenced true smPCR and the overall cost of using smPCR with de novo DNA synthesis since it reduces futile sequencing from 50% (with 1 molecule per well) to 10% (with ˜0.2 molecules/well). A standard 260 nm O.D measurement can be used to determine the optimal concentration.

Even though most of the smPCRs performed using 0.2 molecules per well (i.e. 80% of reactions) have no template, these no-template PCRs are easily identified and distinguished from “true” smPCRs, and their sequencing is avoided. Additionally, the cost of no template PCRs is further diminished by performing the reactions in very low volume (down to 2 ul in standard liquid handling robots). It was also found that RT-PCR can be used to accurately determine the dilution required to dilute the template to the calculated optimal concentration (0.2 molecules per well). A one-time calibration, as described above, allows the routine use of RT-PCR to determine the dilution required before each smPCR experiment. This strategy proved as accurate and as robust as performing the dilution according to a 260 nm O.D measurement and was used throughout the work presented in this paper.

RT-PCR Facilitates the Diagnosis of Faulty Reactions

RT-PCR was used to confirm that the efficiency at which the C-A primer of some embodiments of the present invention amplifies DNA is close to 100%. Given this efficiency, the number of PCR cycles required to reach PCR amplification saturation can be predicted from the initial and typical final template concentrations.

FIG. 7 shows that the number of cycles required for single molecule amplification can be accurately anticipated given the initial and final amount of DNA in a PCR with known amplification efficiency, as for the above described experimental efficiency. The upper curve, labeled “two fold amplification” shows the number of amplified DNA molecules in a PCR reaction that started from a single molecule as a function of cycle number assuming 100% amplification efficiency. The lower curve, labeled “real time PCR”, shows an amplification curve from a smPCR performed with real-time detection. The y-axis for the lower curve is shown to the left and features the number of fluorescent units. The y-axis for the upper curve is shown to the right and features the number of picomoles of DNA formed. The x-axis for both curves relates to the PCR cycle number.

The RT-smPCR results confirm that this prediction is accurate all the way down to single molecule amplification, which displays an amplification curve that is detectable from cycle ˜32 and saturates after ˜42 cycles as described above. This prediction allows real-time determination of whether PCRs are true smPCRs or false positives (e.g. contaminated, actually had many template molecules or primer dimers) since they do not exhibit a typical amplification curve which indicates single molecule amplification, eschewing their further analysis.

Single-Molecule Verification with Random Oligos

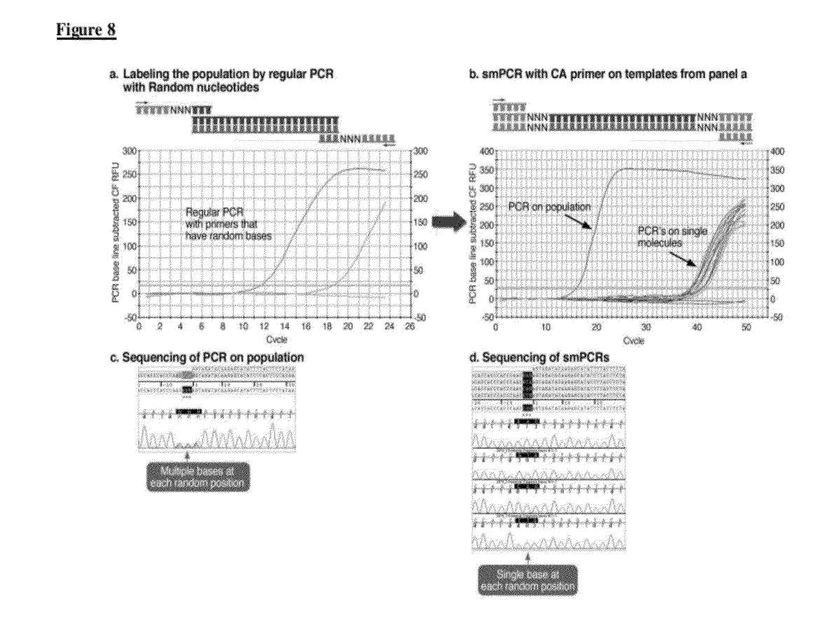

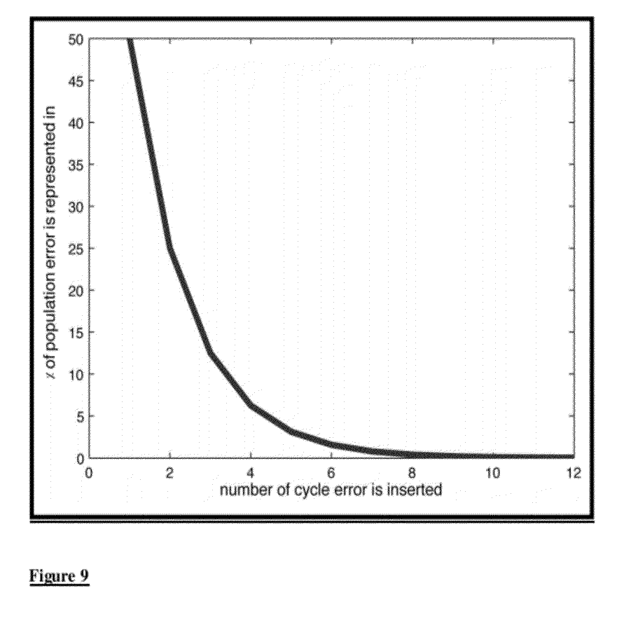

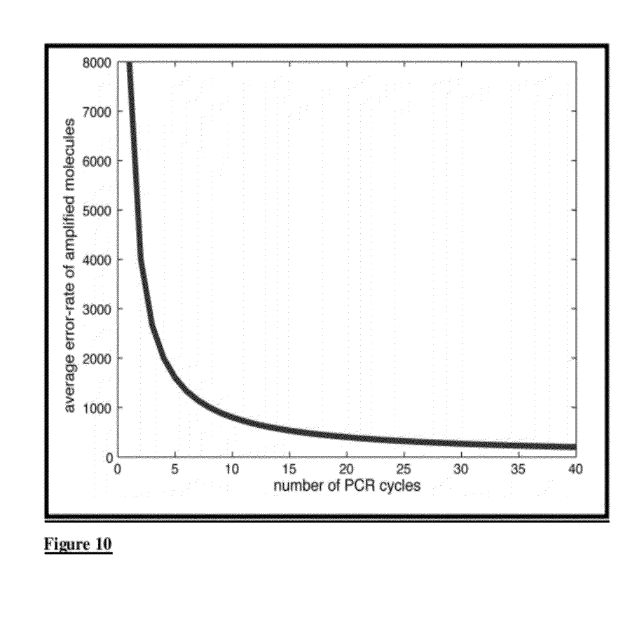

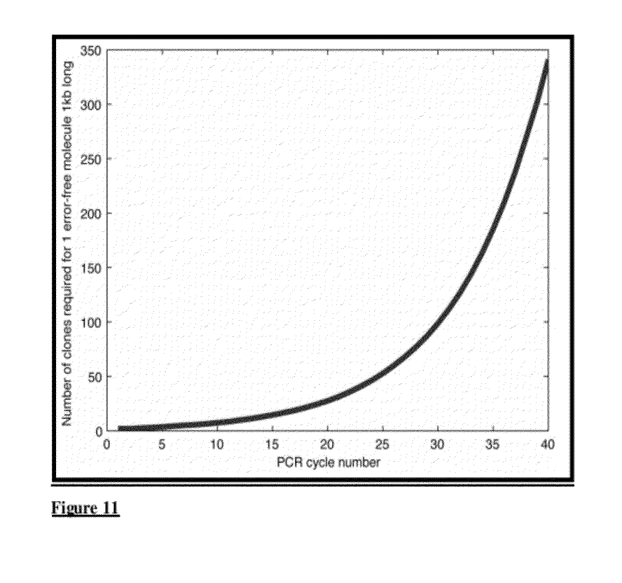

To facilitate the simple identification of rare smPCRs that despite the measures reported above were still not performed on single molecules, another feature is preferably incorporated into this embodiment of the present invention. This feature includes the use of oligos with three random bases at both ends of the synthetic DNA constructs that are to be cloned, effectively bar-coding the molecules with a 4 letter code at 6 positions (4̂6=4096 tags). FIG. 8 shows the use of randomized primers and the results thereof. As shown in FIG. 8A, primers with random bases are inserted into the termini of the molecules by PCR and the reaction is terminated at the exponential phase to avoid hetero-dimers. The upper illustration shows a schematic randomized primer reaction.

FIG. 8B shows that DNA molecules from the right hand PCR curve shown in FIG. 8A are diluted and used as templates for smPCR with the CA primer (PCRs on single molecules). As control a “false positive” smPCR with the same DNA but with many template molecules was also performed. Again, the upper illustration shows a schematic randomized primer reaction.

FIG. 8C shows that the sequencing chromatogram of the “false positive” smPCR from FIG. 8B shows all 4 bases at the 3 random positions, indicating that the reaction was not a true smPCR. FIG. 8D shows that the sequencing chromatograms of 4 different smPCRs from FIG. 8B show only one base call at each of the three random positions, indicating they were true smPCRs.

Overall, sequencing these molecules shows that the sequence at the location of the random bases is always singular in the sequencing of a true smPCR as shown in FIG. 8D and multiple in PCRs performed on >1 template molecules, as shown in FIG. 8C.

Fidelity of Single Molecule Amplification

Errors produced by smPCR pose a minor problem in sequencing and genotyping applications since they can only produce artifacts if inserted during the first few rounds of amplification (11). Errors inserted after the first few cycles (i.e. the remaining ˜36-37 cycles) are represented in a low fraction of the population and are not detectable by sequencing. For example, FIG. 9 shows that the population of molecules featuring such an error is reduced as the cycle number increases during which the error is inserted (ie in later cycles). The y-axis shows the percentage of the population of molecules featuring this error while the x-axis shows the number of the cycle in which the error is inserted.

Nevertheless, errors are inserted during all cycles of smPCR at a fixed rate. For example, FIG. 10 shows the average error-rate of DNA molecules amplified from a single error-free molecule using PCR with Taq polymerase as a function of number of PCR cycles performed. The y-axis shows the average error rate of the amplified molecules while the x-axis relates to the number of PCR cycles.

Although this hardly affects DNA reading applications, for the reasons given above, it dramatically affects DNA writing using smPCR since the smPCR amplified molecules are used as building blocks for further synthesis. Using a standard Taq polymerase with an error-rate of 1/8000 (17) to amplify single error-free DNA molecules results in amplified copies that have an average error rate of 1/200 compared to the original sequence after the 40 PCR cycles required for single molecule amplification, as shown in FIG. 10. This linear increase of error-rate with polymerase cycling results in an exponential increase in the number of clones that have to be sequenced in order to obtain an exact copy of a template molecule 1 Kb long, as shown in FIG. 11. FIG. 11 relates to the average error-rate of DNA molecules amplified from a single error-free molecule using PCR with Taq polymerase as a function of number of PCR cycles performed.

The 800 bp long DNA coding for the GFP from synthetic unpurified oligos was recursively constructed and error corrected using the above described smPCR-based procedure with a Taq DNA polymerase. The clones produced from the uncorrected GFP constructs were sequenced and had an error rate of 1/129, as shown in Table 1 for GFP construction. Table 1 shows a summary of errors from the sequencing of clones (made by the smPCR procedure with Taq) before error correction. Only error-free fragments from them were used for the reconstruction of the full-length molecule.

The error rate of full length error corrected GFP molecules (after reconstruction) with the smPCR procedure was determined by traditional cloning of the error corrected molecules into E. coli and sequencing. The results for the in vitro method were poor in comparison to traditional cloning, as expected, reflecting an error-rate of 1/215, as shown in Table 2 for GFP reconstruction. Table 2A shows the summary of errors from the sequencing of clones (made by the smPCR procedure with Taq) of GFP constructs after error correction. Table 2B shows the summary of errors from the sequencing of clones (made by in vivo cloning) of GFP constructs after error correction.

No error-free GFP molecules were found among the 12 clones, reinforcing the above calculations. The error-corrected clones turned out to be error-prone even though the segments used for their reconstruction were error-free. These segments seemed error free in the sequencing of smPCR clones since most of the errors inserted during smPCR amplification (i.e. during the last ˜37 of the 40 cycles required) are invisible in the sequencing chromatogram. To make sure the errors originated from smPCR and not from the oligos we repeated the exact same error-correction procedure using traditional in vivo cloning of the GFP fragments into E. coli instead of smPCR. As with the smPCR procedure, error-free segments were chosen and used for reconstruction of the target GFP molecule. This control procedure yielded error-free GFP molecules out of almost every clone, as described above.

Therefore, the entire procedure using Taq is less effective for de novo DNA synthesis since the error-rate resulting from smPCR amplification is roughly the error-rate of the synthetic molecules before any error-correction. Moreover, error-correction using smPCR with Taq may even increase the number of clones needed compared to construction with no error-correction, depending on the error-rate of the oligos used, as described in greater detail below.

Nevertheless, technically the procedure was successful (i.e. there were no frame-shifting heteroduplexes, properly calculated limiting dilution, no primer-dimer problems, etc.), indicating that the remaining difficulty is indeed the error rate of the polymerase.

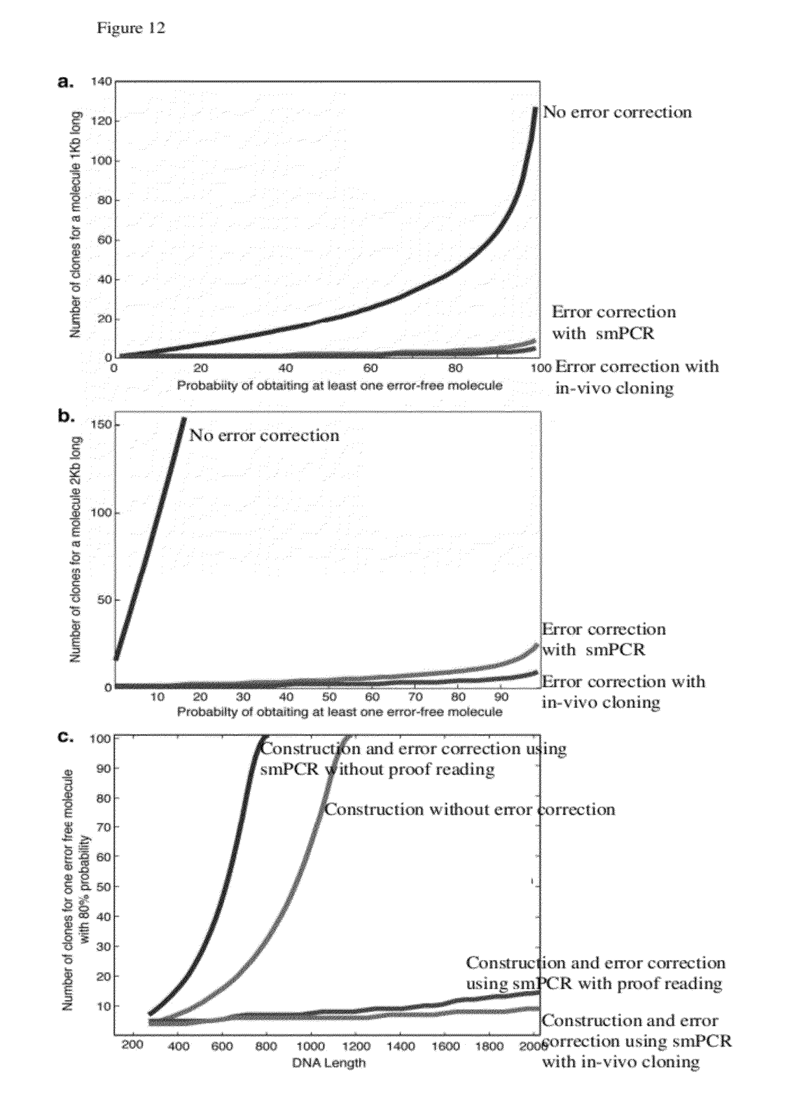

These problems were overcome by selecting appropriate conditions to overcome the problem of the error rate of the polymerase. One optional embodiment of the present invention features a proof reading polymerase to overcome this problem. FIG. 12 shows the results of experiments with such a proof reading polymerase, indicating that error-free molecules are readily cloned using smPCR.

FIGS. 12A (for a 1 Kb molecule) and 12B (for a 2 Kb molecule) show the probability that at least one of the molecules after error correction is error-free as a function of the number of molecules screened. As shown for both Figures, the blue plot indicates no error-correction or error-correction with smPCR using Taq (error-rate 1/200); the green plot shows error-correction with smPCR using a proofreading polymerase; and the red plot shows error-correction with in vivo cloning.

FIG. 12C shows the total number of clones needed for the construction of at least one error-free molecule with 90% probability as a function of the length of the molecule, including clones of construction.

De Novo Synthesis of a 1.8 Kb Mitochondrial DNA Using the smPCR Procedure

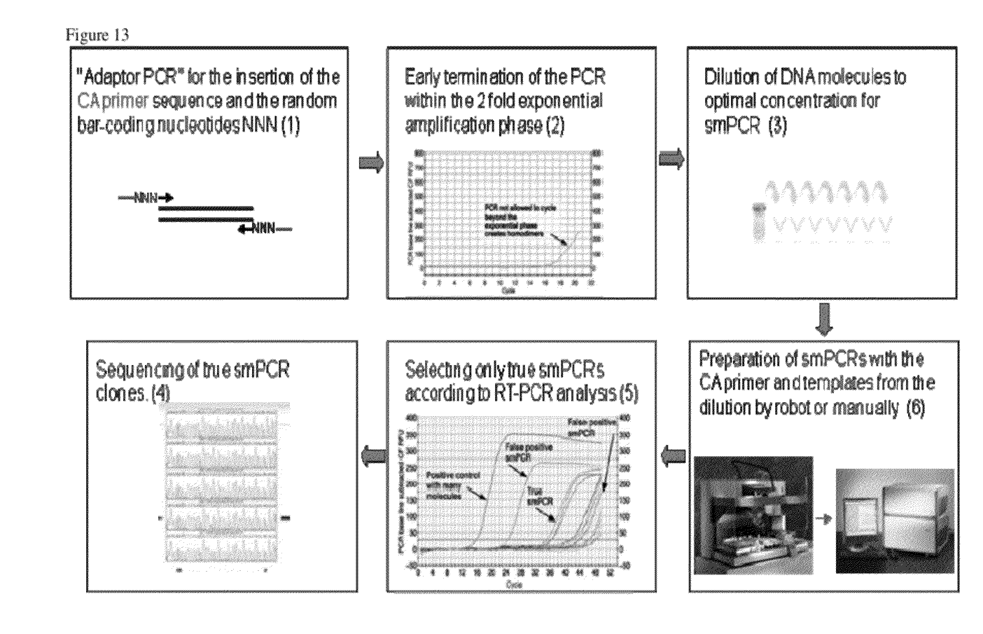

The above described processes were performed in order to construct a 1.8 Kb polynucleotide using the smPCR procedure. FIG. 13 shows an overview of the process. As shown in stage 1, an adaptor PCR is used for the insertion of the CA primer sequence and the random bar-coding nucleotides NNN. In stage 2, early termination of the PCR within the 2 fold exponential amplification phase, in order to obtain all or at least mainly homodimers.

In stage 3, the DNA molecules were diluted to an optimal concentration for smPCR. In stage 4, smPCRs were prepared with the CA primer and templates from the dilution by robot or through manual preparation.

In stage 5, only true smPCRs were selected according to RT-PCR analysis. In stage 6, the true smPCR clones were sequenced.

The procedure was tested by using Accusure, a more accurate (proof-reading) DNA polymerase. The process was used to construct a longer synthetic construct 1.8 Kb long, since a fragment of this length would demonstrate that the procedure can be used for the complete in vitro synthesis and error correction of most synthetic genes. Its synthesis and error correction was conducted as a comparative analysis between the in vitro smPCR-based procedure and an in vivo cloning-based procedure.

Overall, the molecule was constructed from unpurified oligos up to the cloning phase and then the error-correction process was split into two separate and parallel courses executed side-by-side using the same starting material, one with smPCR and the other with in vivo cloning.

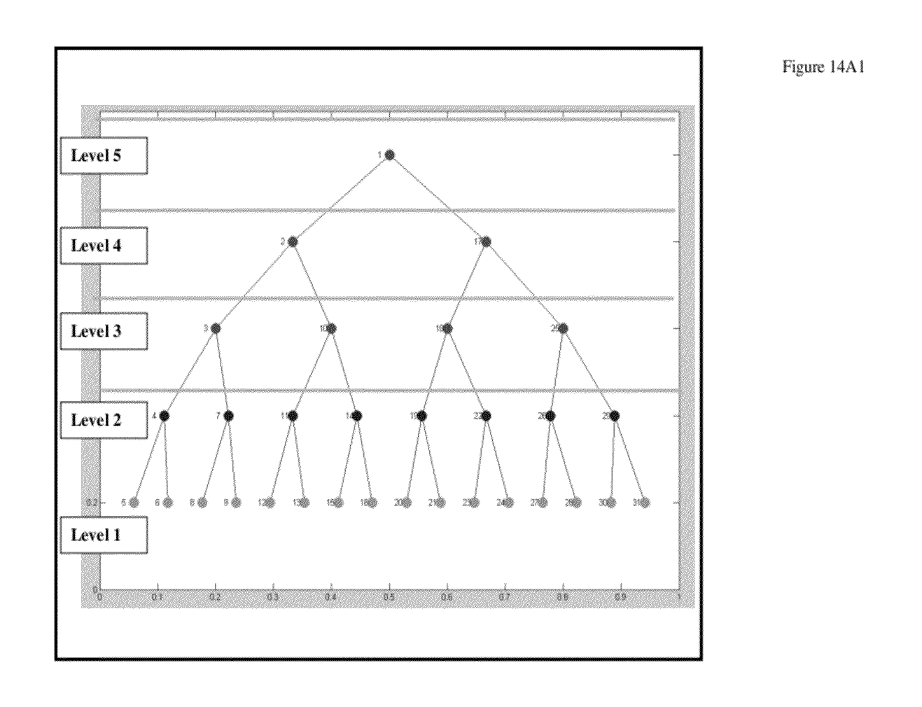

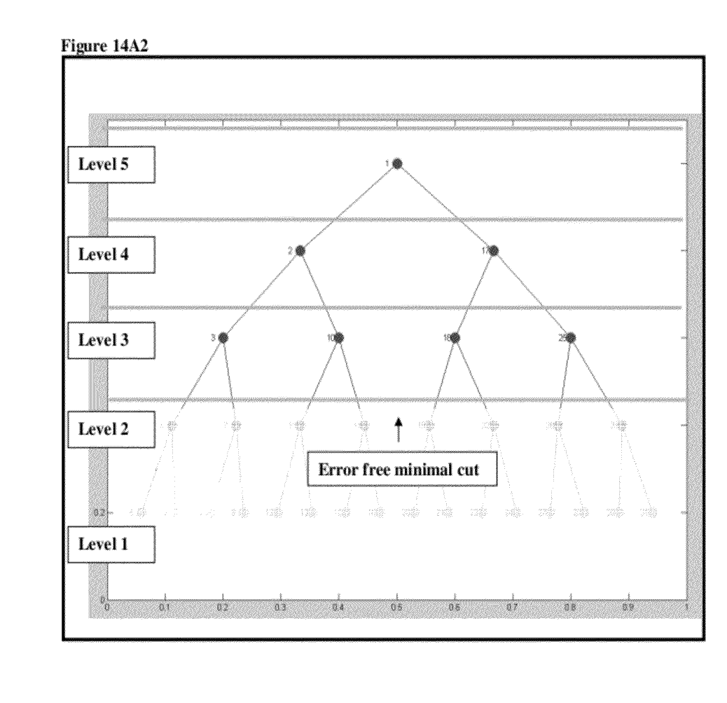

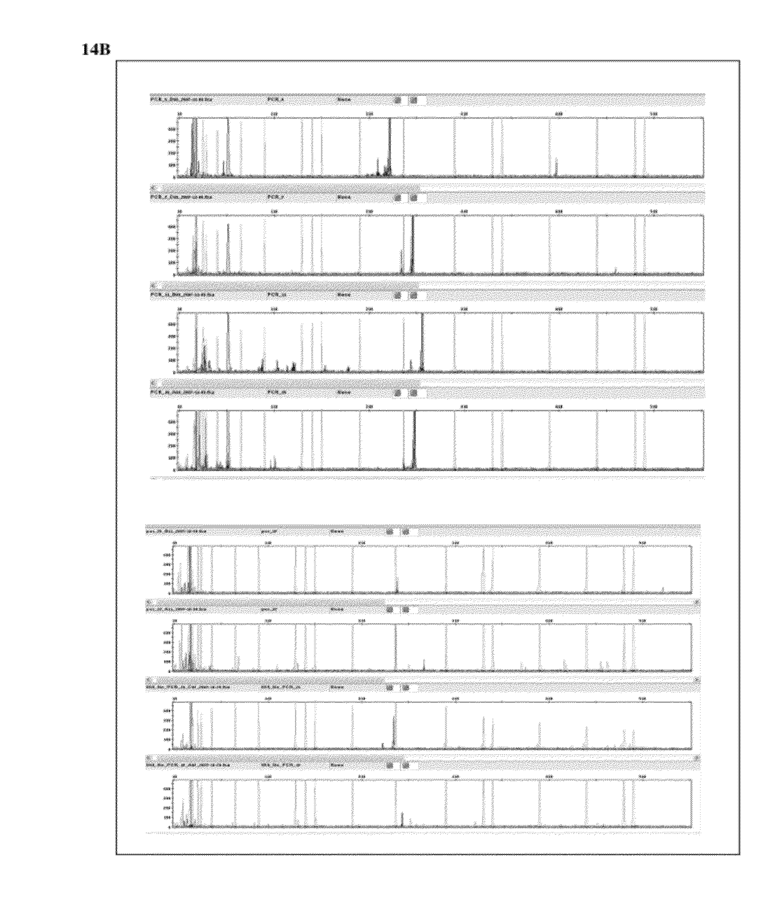

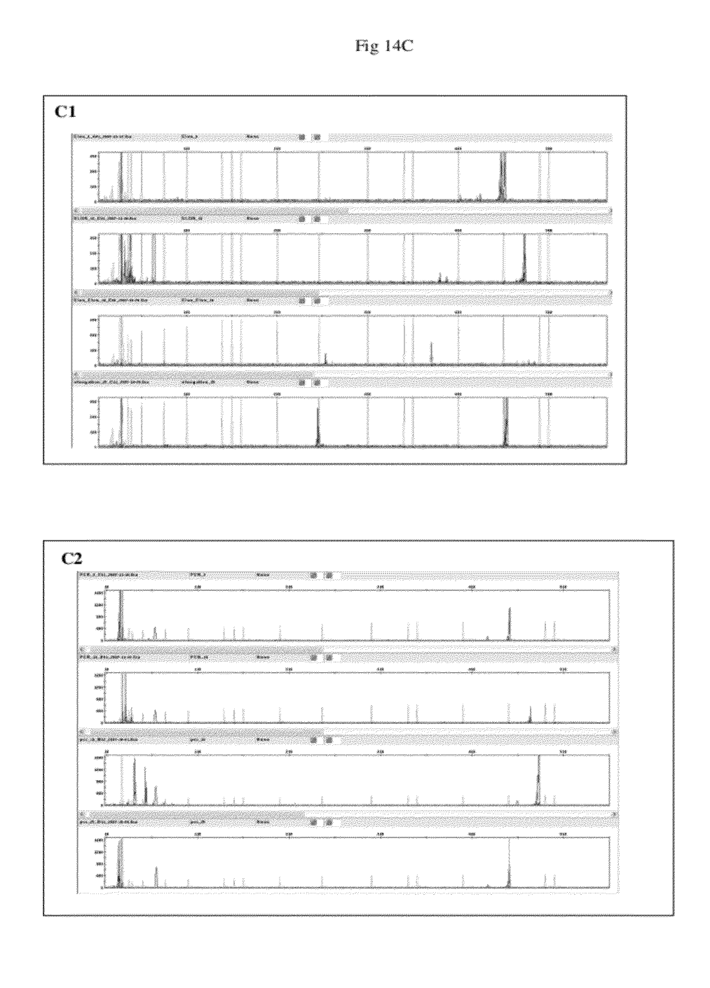

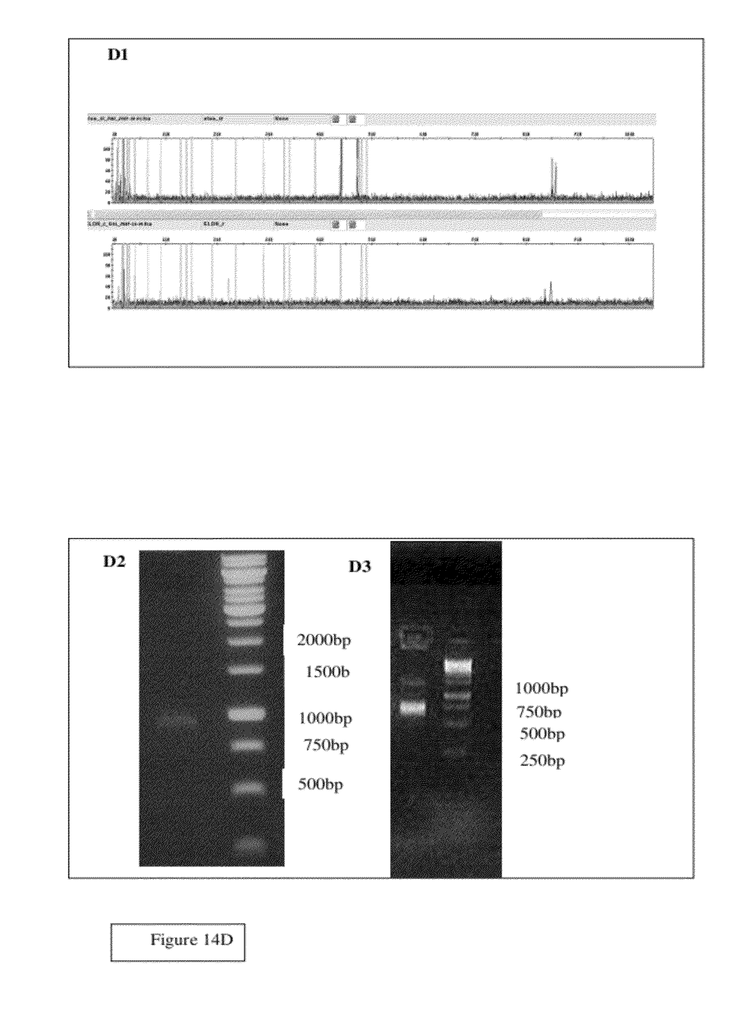

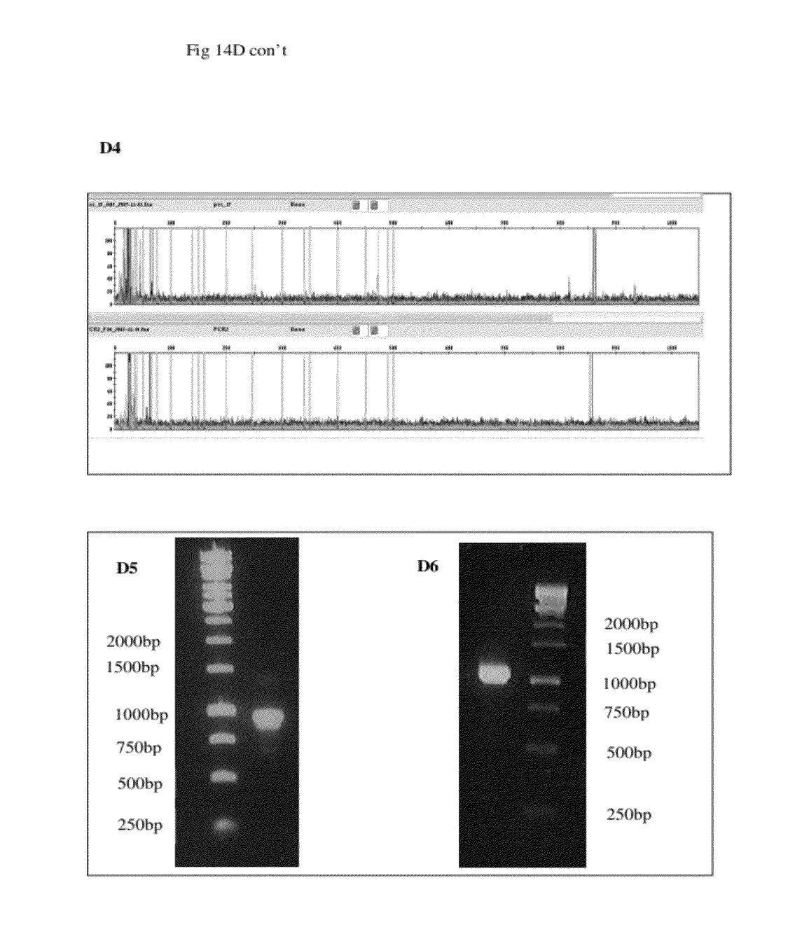

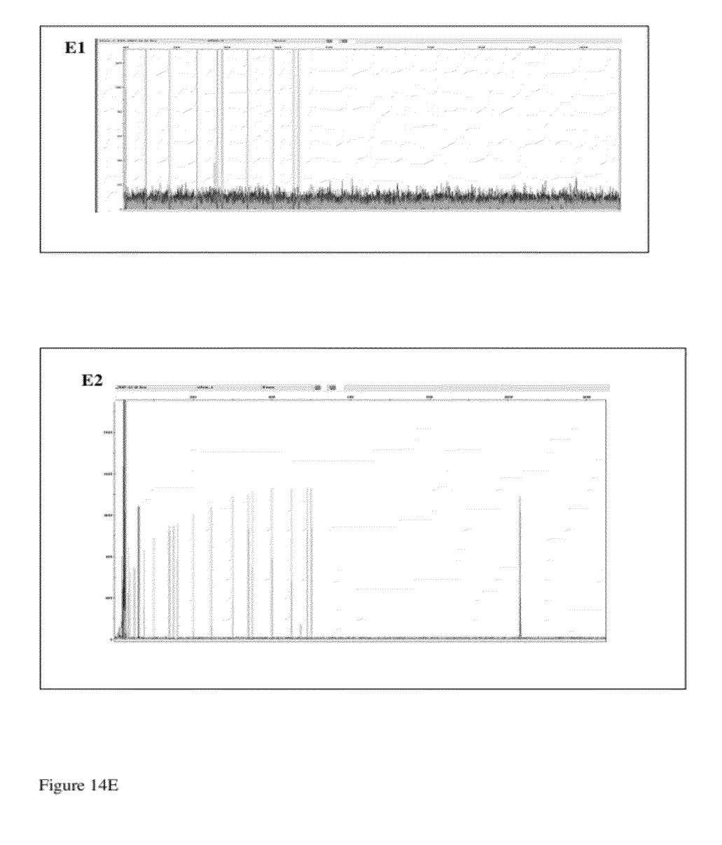

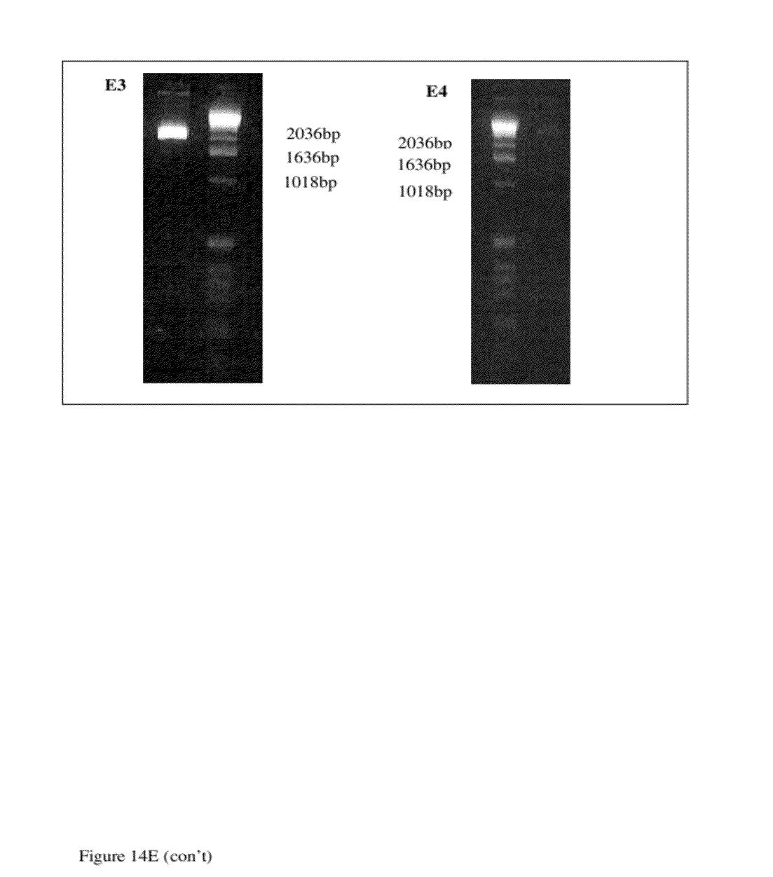

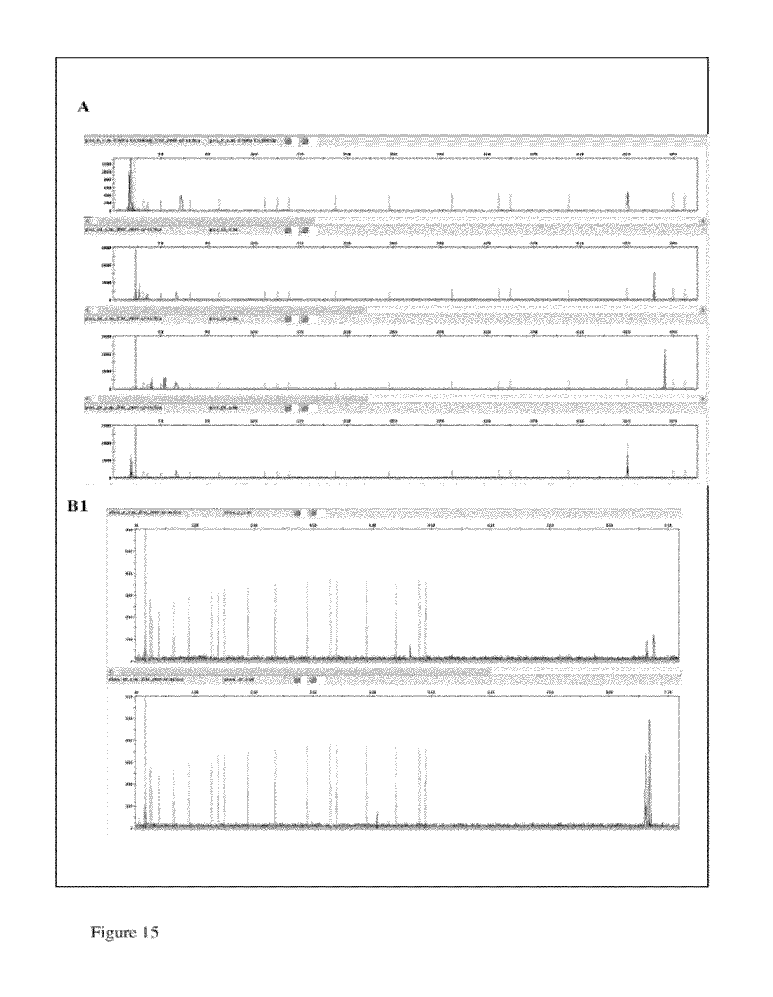

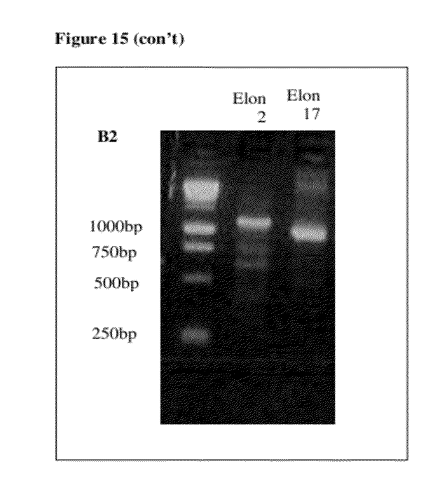

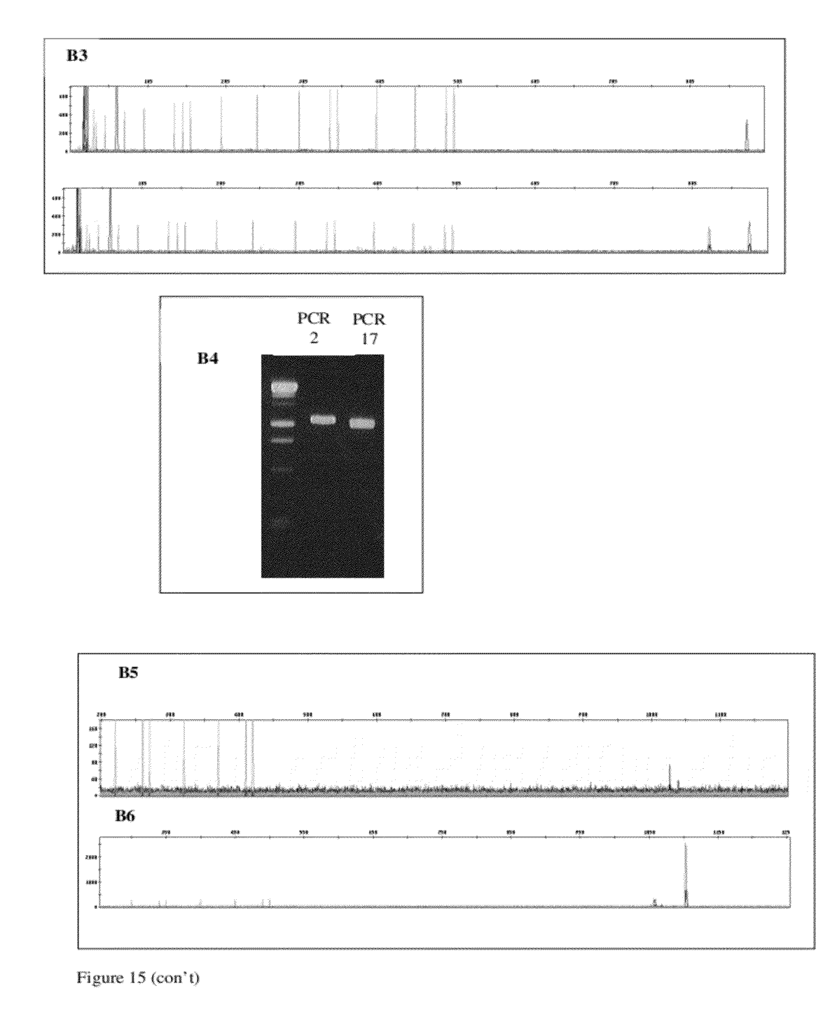

Turning now to the construction process, as shown in FIG. 14A, the construction protocol of the molecule is represented as a tree divided to levels of construction. Fragments that occur during construction and reconstruction are represented as the numbered nodes in the tree. This numbering is used for the description of the other parts of FIG. 14, as well as for FIG. 15. It should be noted that FIG. 14A1 shows the process as performed for FIG. 14, while FIG. 14A2 shows the process as performed for FIG. 15, with the addition of the error free minimal cut, indicated by an arrow.

FIG. 14B shows the PCRs of construction level 1. The capillary electrophoresis (CE) results are of PCRs of the following nodes, from top to bottom: 4, 7, 11, 14, 29, 22, 26, 19. Their expected sizes in base pairs (bp) are, from top to bottom: 221, 219, 221, 217, 218, 219, 219, 220.

FIG. 14C1 shows the results of the elongations from construction level 2. The CE results show elongations of the following nodes, from top to bottom: 3, 10, 18, 25. Their expected sizes in base pairs are, from top to bottom: 440, 438, 439, 437.

FIG. 14C2 shows PCRs of construction level 2. The CE results are related to PCRs of the following nodes, from top to bottom: 3, 10, 18, 25. Their expected sizes in base pairs are, from top to bottom: 440, 438, 439, 437.

FIG. 14D1 shows the results of the elongation of construction level 3. The CE results show elongation of the following nodes, from top to bottom: 17, 2. Their expected sizes in base pairs are, from top to bottom: 876, 878.

FIG. 14D2 shows the results of the elongation of node 2 from construction level 3, as determined according to gel electrophoresis, due to size restrictions for CE. The expected size in base pairs is: 878.

FIG. 14D3 shows the results of the elongation of node 17 from construction level 3, as determined according to gel electrophoresis, due to size restrictions for CE. The expected size in base pairs is: 876.

FIG. 14D4 shows the results of PCRs from construction level 3. The CE results show the PCRs of the following nodes, from top to bottom: 17, 2. Their expected sizes in base pairs are, from top to bottom: 876, 878.

FIG. 14D5 shows the result of the PCR of node 2 from construction level 3 (again as performed by gel electrophoresis due to size constraints). The expected size in base pairs is: 878.

FIG. 14D6 shows the result of the PCR of node 17 from construction level 3 (again as performed by gel electrophoresis due to size constraints). The expected size in base pairs is: 876.

FIG. 14E1 shows the results of the elongation of node 1 from mitochondria construction level 4 (again as performed by gel electrophoresis due to size constraints). The expected size in base pairs is: 1754.

FIG. 14E2 shows the results of PCR of node 1 from mitochondria construction level 4 (again as performed by gel electrophoresis due to size constraints). The expected size in base pairs is: 1754.

FIG. 14E3 shows the results of PCR of node 1 from mitochondria construction level 4 (as performed by gel electrophoresis due to size constraints). The expected size in base pairs is: 1754.

FIG. 14E4 shows the results of elongation of node 1 from mitochondria construction level 4 (as performed by gel electrophoresis due to size constraints). The expected size in base pairs is: 1754.

FIG. 15 relates to CE and Gel fragment analysis of reactions from the error corrective reconstruction using the smPCR protocol.

FIG. 15A shows PCRs from reconstruction level 2; the CE results show PCR of the following nodes, from top to bottom: 3, 10, 18, 25. Their expected sizes in base pairs are, from top to bottom: 440, 438, 439, 437.

FIG. 15B1 shows the results of elongation from reconstruction level 3. The CE results show elongation of the following nodes, from top to bottom: 2, 17. Their expected sizes in base pairs are, from top to bottom: 878, 876.

FIG. 15B2 shows the results of elongation from reconstruction level 3 (as performed by gel electrophoresis due to size constraints). The gels show elongation of the following nodes, from top to bottom: 2, 17. Their expected sizes in base pairs are, from top to bottom: 878, 876.

FIG. 15B3 shows the results of PCRs from reconstruction level 3. The CE results show PCR of nodes 2 and 17 from top to bottom. Expected sizes in by from top to bottom are: 878, 876.

FIG. 15B4 shows the results of PCRs from reconstruction level 3 (as performed by gel electrophoresis due to size constraints). The gels show PCR of nodes 2 and 17 from top to bottom. Expected sizes in by from top to bottom are: 878, 876.

FIG. 15B5 shows the CE results of elongation from reconstruction level 4, node 1. Expected size in by is: 1754.

FIG. 15B6 shows the CE results of PCR from reconstruction level 4, node 1. Expected size in by is: 1754.

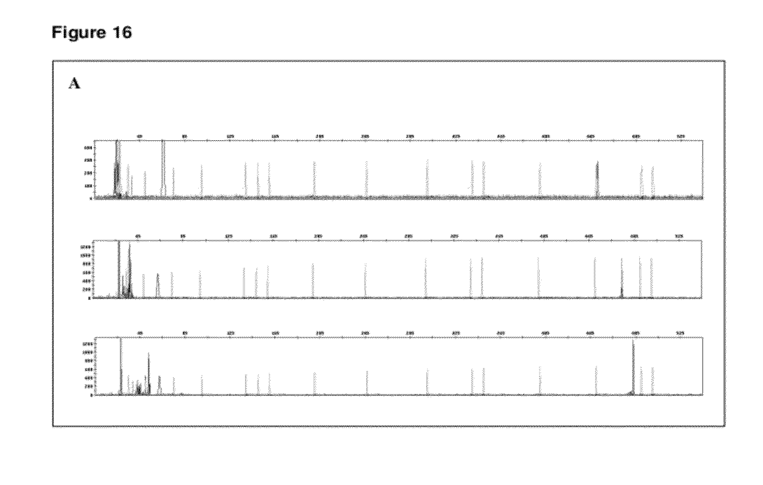

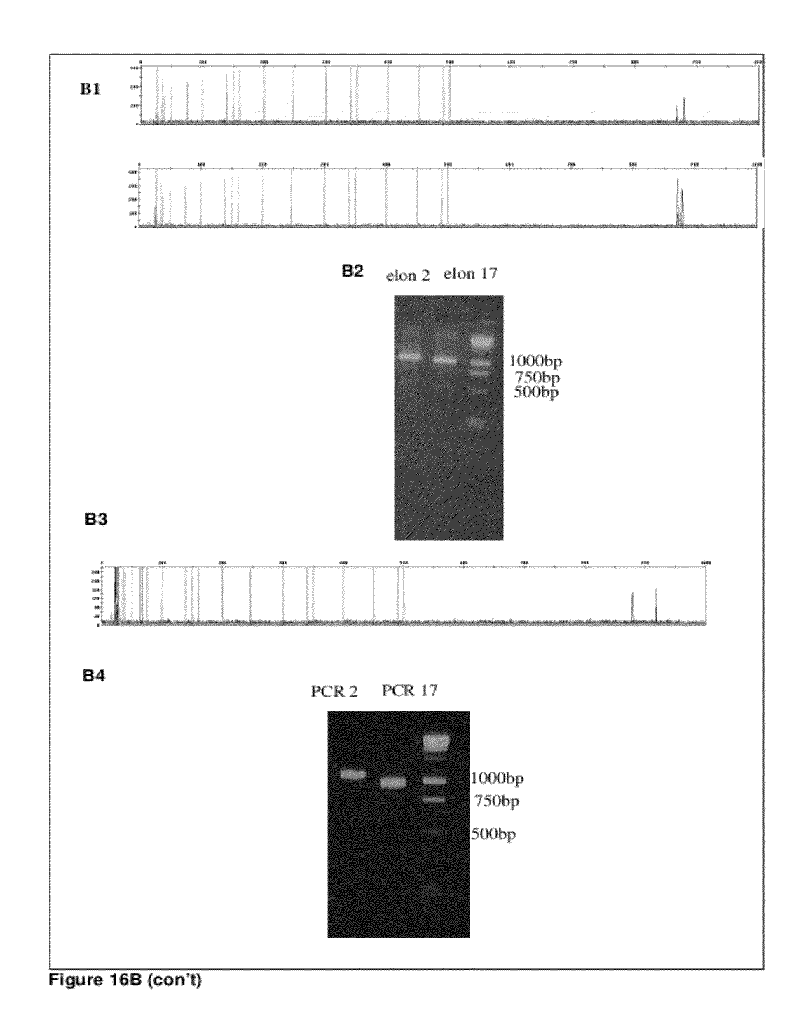

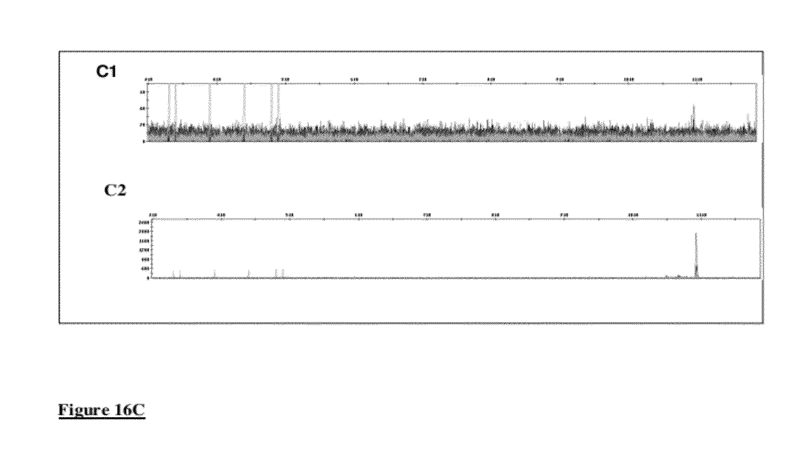

FIG. 16 shows the results of CE and gel fragment analysis of reactions from the error corrective reconstruction using in vivo cloning.

FIG. 16A shows the results of PCR for mitochondria reconstruction clone level 2, from top to bottom: 3, 10, 18. Expected sizes in by from top to bottom are: 440, 438, 439.

FIG. 16B1 shows the results of elongation of reconstruction level 3. CE results are from top to bottom of nodes: 2, 17. Expected sizes in by from top to bottom are: 878, 876.

FIG. 16B2 shows the results of elongation of reconstruction level 3. Gels are from top to bottom of nodes: 2, 17. Expected sizes in by from top to bottom are: 878, 876.

FIG. 16B3 shows the results of PCRs of reconstruction level 3. CE results are for node: 2. Expected size in bp: 878.

FIG. 16B4 shows the results of PCRs of reconstruction level 3. Gels are from top to bottom of nodes: 2, 17. Expected sizes in by from top to bottom are: 878, 876.

FIG. 16C1 shows the results of elongation of reconstruction level 4. The CE results are for node 1. Expected size in by is: 1754.

FIG. 16C2 shows the results of PCR of reconstruction level 4. The CE results are for node 1. Expected size in by is: 1754.

Clones generated by both methods before error-correction were sequenced and their error-rate was the same, as shown in Tables 3 and 4, for Mitochrondria construction. Table 3 shows the summary of errors from the sequencing of clones (made by in vivo cloning) of the 1.8 Kb mitochondrial fragment before error correction. Table 4 shows the summary of errors from the sequencing of clones (made by the smPCR procedure) of the 1.8 Kb mitochondrial fragment before error correction. It is expected that the same error-rate would be obtained for both, reflecting the error-rate of the synthetic oligos used in synthesis (4, 15).

As previously described, the same set of error-free of segments (i.e. the minimal cut) was identified in both sets of clones and used them to reconstruct the target 1.8 Kb molecule twice, once from each set of clones and using the exact same protocol for reconstruction. Once reconstructed from error-free segments, the two 1.8 Kb synthetic constructs were cloned into E. coli and sequenced in order to evaluate their error-rate.

Target constructs from the smPCR procedure had an error-rate of 1/1128 (Table 6, Mitochondria construction) (there is no reference to compare this with as the Accusure error-rate is not known), giving a ˜6 fold improvement compared to the same procedure using Taq polymerase (See GFP results) and to the error-rate of initial uncorrected synthetic DNA. Table 6 shows a summary of errors from the sequencing of clones (made by in vivo cloning) of the 1.8 Kb mitochondrial fragment after error correction (using the smPCR procedure).

Error-free synthetic 1.8 Kb target molecules were easily obtained from a small number of clones with this improved error-rate (see previously described FIG. 12). The control in vivo cloning procedure also yielded error-free clones at an error-rate of 1/2193 (Table 5, Mitochondria construction). Table 5 shows a summary of errors from the sequencing of clones (made by in vivo cloning) of the 1.8 Kb mitochondrial fragment after error correction (using in vivo cloning).

The 1/1128 error rate obtained using a proof-reading enzyme for the smPCR-procedure is sufficient for the synthesis of most genes with a reasonable number of clones (see previously described FIG. 12). This error-rate is a result of two factors, namely the errors inserted during smPCR amplification and errors inserted during the PCR amplifications required for the reconstruction process. The 1/2193 error rate obtained from error correction using traditional cloning is most probably largely due to the errors inserted during the PCR amplifications required for reconstruction since in vivo amplification of DNA is very accurate. Although the overall error rate of the procedure using in vivo cloning is better than with the in vitro cloning presented here, this ˜2 fold difference in error rates only slightly affects the number of clones required for obtaining error-free synthetic molecules of most genes (see previously described FIG. 12). In general, the probability that a given synthesis process yields error-free molecules largely depends on the number of clones that are sequenced.

For example, even synthesis without error correction can, in principle, produce error-free clones with high probability if a very large number of clones are screened. Conversely, the same process is unlikely to produce error-free molecules if a small number of clones are screened. Therefore, it is useful to describe for different synthesis methods how the number of sequenced clones influences the probability of obtaining error-free clones and, more practically, vice versa, how the required probability of success of obtaining error-free clones determines the number of clones that one should sequence (see previously described FIGS. 12A and B).

The test results show the smPCR procedure according to some embodiments of the present invention is highly comparable to traditional cloning. Even with high success requirements (90% probability) the difference between the smPCR procedure and traditional cloning is negligible up to the 2 Kb range at least (see previously described FIGS. 12A and B). For example, finding error-free fragments after error correction 1 kb and 2 Kb long with probability of at least 90% requires only 4 and 8 clones respectively after using our smPCR method compared to 2 and 3 clones after using in vivo cloning.

Discussion

The results described herein show that, even though smPCR has typically been used in DNA reading applications to date (11-14), by following the procedures described herein (as non-limiting examples only of the present invention), it can also be used for the typically cloning intensive de novo DNA writing (construction). For the first time a general method for the synthesis of long synthetic fragments was demonstrated from unpurified oligos completely in vitro. The entire method as reported here is highly accessible to every lab since it is performed using off-the-shelf reagents, standard lab equipment and requires no special expertise.

The total construction and error correction of synthetic error free fragments of at least ˜2 Kb can be made from a small number of clones using our in vitro method and that these results are comparable to construction using traditional in vivo cloning (see previously described FIG. 12C). The use of other thermostable enzymes with improved fidelity (18) is expected to enable synthesis of even larger synthetic DNA molecules using the same or similar procedure. Alternatives to high fidelity DNA amplification with thermostable polymerases, for example mesophilic amplification based on the isothermal strand displacement polymerization activity of the phi29 polymerase may also be considered in the future. The phi29 polymerase, already shown to be useful in the amplification of single DNA molecules (19) is comparable in accuracy to high fidelity thermostable polymerases (20), however its integration into a DNA synthesis scheme is not straightforward.

Although these experiments demonstrate the integration of in vitro cloning based on smPCR with a specific DNA synthesis method, the present invention is not limited to this implementation; indeed, these embodiments of the present invention may optionally be used as an alternative to the cloning phase of other DNA synthesis methods as well and for the cloning of synthetic DNA in general. Cloning of synthetic DNA molecules using smPCR is more rapid (˜3 hours), it is amenable to automation (using standard liquid handling robots) and scalable (using 96 or 384 well PCR plates), whereas traditional cloning is time consuming (˜1-2 days), manual labor intensive and difficult to automate.

A major requirement for automated DNA synthesis is robustness and reproducibility. Performing PCR directly on colonies is that it is not as robust and reproducible as traditional production and purification of plasmids. Additionally, although automated colony picking does exist it requires relatively expensive specialty equipment, while the process reported in this manuscript only requires standard lab equipment and turned out to be a highly robust and reproducible process.

Furthermore, automation of traditional cloning doesn't sum up to only automated colony picking. It also requires inoculation of bacteria in sterile conditions into a Petri dish and overnight growing of colonies. These are difficult to automate and time consuming, respectively. It should be noted that automated colony picking may be substituted by in vivo cloning-by-dilution, but this may hold difficulties of its own such as the absence of selection for blue/white colonies which helps avoid futile sequencing.

In any case, all this is preceded by the process of inserting DNA into cells (the transformation itself) which may be performed in 96-well electro-poration devices or by heat shock but usually requires some manual labor and is not easily performed in an automated robotic setup. Moreover, the new procedure described here does not require the use of cells of any kind and therefore reduces potential biohazards associated with replicating specific DNA fragments in vivo, for example by not overusing antibiotic resistance for cloning, and also allows processing of fragments that are difficult to replicate in vivo.

Although these experiments describe a small scale process, clearly these embodiments of the present invention could easily be scaled up and automated. The method's simplicity, rapidness and amenability to automation make it a possible alternative to traditional cloning practice in DNA synthesis.

### Example 2

### Bar Coding Molecules for Polynucleotide Construction

According to some embodiments, the present invention provides a method, system and apparatus for bar coding molecules for polynucleotide construction. By “bar coding” it is meant that a “code” of nucleotides is added to the polynucleotides during construction, in order to identify these polynucleotides (for example, to ensure that a particular polynucleotide has been successfully amplified and/or otherwise detected.

To facilitate bar-coding, preferably oligos with a plurality and preferably three random bases are used at least one, but more preferably at both ends of the synthetic DNA constructs that are to be cloned, effectively bar-coding the molecules with a 4 letter code at 6 positions (4̂6=4096 tags) in the case of oligos having three random bases used at both ends of the constructs. Preferably, primers with random bases are inserted into the termini of the molecules by PCR; any type of amplification may optionally be used with such bar coding.

Without wishing to be limited, this process may optionally be used for many applications. For example, it may optionally be used to label polynucleotides within a large population, in order to be able to detect each such polynucleotide separately or by category (or group). Optionally and preferably, such detection may also optionally be used to thereby separate out a single polynucleotide or a category of such polynucleotides. Furthermore, optionally the process may be used to determine the origin of a particular polynucleotide or group thereof within a larger mixture of molecules. Thus, the bar code may optionally be used for detection, identification and/or separation of a polynucleotide (or group thereof) from a plurality of polynucleotides.

### Example 3

### Determining Dilution for Single Molecule Amplification

According to some embodiments, the present invention provides use of Real-Time PCR(RT-PCR) for determining the dilution required for single molecule amplification. As described herein in a non-limiting example, RT-PCR can be tracked to determine the dilution required for a single molecule to be amplified. Specifically, the number of cycles required for single molecule amplification can be accurately anticipated given the initial and final amount of DNA in a PCR with known amplification efficiency.

For example, a process for PCR having a known amplification efficiency could be used to amplify a DNA molecule. If the initial amount of the DNA molecule is known, then the known amplification efficiency, the dilution and the initial amount in combination could optionally be used to determine the number of cycles required for single molecule PCR. Alternatively or additionally if the amplification efficiency, the dilution and the initial amount in combination are known, then it is possible to determine the amount of polynucleotide obtained at each cycle. Alternatively, if the amplification efficiency, the dilution, the number of cycles and the final amount are known, then the initial amount may optionally be determined.

### Example 4

### Determining Correct SNP Patterns in a Population

According to some embodiments, the present invention provides a method for determining the correct SNP patterns in a population, by enabling actual SNPs at a plurality of different locations to be detected. Currently, by using in vivo cloning with bacterial cells for example, it is possible to detect SNPs but it is not possible to determine the correct pattern, since the bacterial cells may cause SNP combinations to appear in the cloned material which do not occur in the population.

By contrast, according to some embodiments of the present invention, smPCR with single stranded polynucleotides as performed according to the present invention detects the true pattern of SNPs and does not generate new (false) combinations of SNPs at a plurality of locations. Thus, it is possible to automatically detect the correct SNP patterns within a population and/or to compare such patterns between populations.

Tables

TABLE 1
GFP construction

| Reference | | 002 | 001 | 004 | 005 | 006 | 007 | 008 | 009 | 010 | 014 | 015 | 016 | 017 | 018 | Total |
|---|---|---|---|---|---|---|---|---|---|---|---|---|---|---|---|---|
| 1 | G | | | | | | | | | C | | | | | | 1 |
| 3 | A | | | | | | | | | : | | | | | | 1 |
| 4 | T | | | | | | | | | : | | | | | | 1 |
| 9.1 | : | | | | | | | | C | | | | | | | 1 |
| 10 | G | | | | | | | | | A | | | | | | 1 |
| 19 | C | | | | | | | | : | | | | | | | 1 |
| 48 | C | | | | | : | | | | | | | | | | 1 |
| 49 | C | T | T | | | : | | | | | | | | | | 3 |
| 50 | G | | | | | : | | | | | | | | | | 1 |
| 51 | G | | | | | : | | | | | | | | | | 1 |
| 52 | G | | | | | : | | | | | | | | | | 1 |
| 53 | G | | | | | : | | | | | | | | | | 1 |
| 61 | C | | | | | | | | | | T | | | | | 1 |
| 65.1 | : | | | | | | C | | | | | | | | | 1 |
| 71 | G | | | | | | | | | | | | | T | | 1 |
| 73 | G | | : | | | | | | | | | | | | | 1 |
| 74 | C | | : | | | | | | | | | | | | | 1 |
| 75 | T | | : | | | | | | | | | | | | | 1 |
| 76 | G | | : | | | | | | | | | | | | | 1 |
| 77 | G | | : | | | | | | | | | | | | | 1 |
| 78 | A | | : | | | | | | | | | | | | | 1 |
| 86 | G | | A | | | | | | | | | | | | | 1 |
| 100 | G | | | | | | | | | | | | A | | | 1 |
| 104.1 | : | | | | | | A | | | | | | | | | 1 |
| 123 | A | | G | | | | | | | | | | | | | 1 |
| 124 | G | | | | | | | | : | | | | | | | 1 |
| 125 | G | | | | | | | | : | | | | | | | 1 |
| 126 | G | | | | | | | | : | | | | | | | 1 |
| 127 | C | | | | | | | | : | | | | | | T | 2 |
| 128 | G | | | | | | | | : | | | | | | | 1 |
| 129 | A | | | | | | | | : | | | | | | | 1 |
| 139.1 | : | | | | | | | | | | | | | C | | 1 |
| 141 | G | | | | | : | | | | | | | | | | 1 |
| 189 | C | | | | | | | | | | | T | | | | 1 |
| 207 | C | | | | | | | | | | | | T | | | 1 |
| 222 | G | | | | | | : | | | | | | | | | 1 |
| 240.1 | : | | | | | | | | | | | | G | | | 1 |
| 246 | C | | | | | | | | T | | | | | | | 1 |
| 247 | C | | | | | : | | | | | | | | | | 1 |
| 248 | G | | | | | | | | | | | : | | | | 1 |
| 259 | G | | | | | | | | | | | | : | | | 1 |
| 260 | C | | | | | | | | | | | | : | | | 1 |
| 272 | T | | | | C | | | | | | | | | | | 1 |
| 289 | C | | | | | : | | | | | | | | | | 1 |
| 290 | G | | | | | : | | | | | | A | | | | 2 |
| 292 | A | | | | | : | | | | | | | | | | 1 |
| 303 | A | | | C | | | | | | | | | | | | 1 |
| 305 | G | | | | | | | | | A | | | | | | 1 |
| 306 | A | G | | | | | | | | | | | | | | 1 |
| 317 | T | | | C | | | | | | | | | | | | 1 |
| 344 | A | | | G | | | | | | | | | | | | 1 |
| 364 | C | | | | T | | | | | | | | | | | 1 |
| 388.1 | : | | | | | | | | C | | | | | | | 1 |
| 389.1 | : | | | | | A | | | | | | | | | | 1 |
| 406 | C | : | | | | | | | | | | | | | | 1 |
| 470 | G | | | | | | T | | | | | | | | | 1 |
| 482 | G | : | | | | | | | | | | | | | | 1 |
| 493 | G | | | A | | | | | | | | | | | | 1 |
| 501 | G | | | | | | | | | | | | | | : | 1 |
| 546 | G | | | | | A | | | | | | | | | | 1 |
| 576 | A | | | | | | | | G | | | | | | | 1 |
| 594 | G | | | | | : | | | | | | | | | | 1 |
| 594.1 | : | | | G | | | | | | | | | | | | 1 |
| 598.1 | : | | | | | | | | | | | | C | | | 1 |
| 606 | T | C | | C | | | | | | | | | C | C | | 4 |
| 615 | A | | | | | | | : | | | | | | | | 1 |
| 626 | A | G | | | | | | | | | | | | | | 1 |
| 639 | C | | | | | | | T | | | | | | | | 1 |
| 669 | A | | G | | | | | | | | | | | | | 1 |
| 675 | T | | | | | | | | | | C | | | | | 1 |
| 686.1 | : | | | | | | G | | | | | | | G | | 2 |
| 704 | G | | | | | | | | | | | | | A | | 1 |
| 719 | A | G | | | | | | | | | | | | | | 1 |
| 720 | T | | | | | | C | | | | | | | | | 1 |
| 738 | A | | | | | | | G | | | | | | | | 1 |

| | | | | | | | | | | | | | | | |
|---|---|---|---|---|---|---|---|---|---|---|---|---|---|---|---|
| Total | 7 | 10 | 6 | 2 | 14 | 6 | 3 | 11 | 5 | 2 | 3 | 7 | 5 | 2 | 83 |

TABLE 2A
GFP reconstruction: Summary of errors from the sequencing of clones
(made by the smPCR procedure with Taq) of GFP constructs after
error correction

| Reference | | 001 | 003 | 004 | 010 | 012 | 015 | 018 | Total |
|---|---|---|---|---|---|---|---|---|---|
| 46 | C | | A | | | | | | 1 |
| 61 | C | | | A | A | | | | 2 |
| 95 | C | A | | | | | | | 1 |
| 103 | C | | | | A | | | | 1 |
| 116 | G | | | | | | T | | 1 |
| 183 | C | | | | A | | | | 1 |
| 184 | C | | | | | | A | | 1 |
| 190 | C | | | | A | | | | 1 |
| 200 | C | | | A | | | | | 1 |
| 214 | G | T | | | | | | | 1 |
| 217 | C | | | | | | A | | 1 |
| 446 | G | T | | | | T | | | 2 |
| 496 | G | | | | | | | T | 1 |
| 517 | C | | | | A | | | | 1 |
| 542 | G | | | | | T | | | 1 |
| 563 | C | A | | | | | | | 1 |
| 580 | C | | | | | | A | | 1 |
| 610 | C | | | | | | A | | 1 |
| 632 | C | | | A | | | | | 1 |
| 636 | C | A | | | | | | | 1 |
| 671 | C | A | | | | | | | 1 |
| 722 | G | | | | | T | | | 1 |
| 725 | G | | | | | T | | | 1 |

| | | | | | | | | |
|---|---|---|---|---|---|---|---|---|
| Total | 6 | 1 | 3 | 5 | 4 | 5 | 1 | 25 |

TABLE 2B
GFP reconstruction

| | Reference | | | | | | |
|---|---|---|---|---|---|---|---|
| | 80001 | 80009 | 80013 | 80015 | 80017 | 80019 | Total |
| Total | 0 | 0 | 0 | 0 | 0 | 0 | 0 |

TABLE 3
Mitochondria construction - Summary of errors from the sequencing of
clones (made by in vivo cloning) of the 1.8 Kb mitochondrial fragment before error
correction

| Reference | | 03 | 04 | 05 | 06 | 07 | 08 | 09 | 10 | 11 | 12 | 13 | 15 | 16 | 17 | 18 | 21 | 02 | Total |
|---|---|---|---|---|---|---|---|---|---|---|---|---|---|---|---|---|---|---|---|
| 2 | G | | | | | T | | | | | | | | | | | | | 1 |
| 4 | A | | | | | C | | | | | | | | | | | | | 1 |
| 8 | A | | | | | : | | | | | | | | | | | | | 1 |
| 9 | C | | | | | T | | | | | | | | | | | | | 1 |
| 22.1 | : | | | | | | | | | T | | | | | | | | | 1 |
| 31 | A | | | | | | T | | | | | | | | | | | | 1 |
| 36 | T | | | | | : | | | | | | | | | | | | | 1 |
| 37 | T | | | | | : | | | | | | | | | | | | | 1 |
| 38 | T | | | | | : | | | | | | | | | | | | | 1 |
| 39 | T | | | | | : | | | | | | | | | | | | | 1 |
| 41 | A | | | | | : | | | | | | | | | | | | | 1 |
| 45 | C | | | | | | | | | | | T | | | | | | | 1 |
| 63 | A | | | | | | | | | | | | | | | C | | | 1 |
| 67 | A | | | | | | | | | | : | | | | | | | | 1 |
| 69 | T | | | | | | C | | | | | | | | | | | | 1 |
| 73 | T | | | | | C | | | | | | | | | | | | | 1 |
| 76 | G | | | | | | | | | | | | | T | | | | | 1 |
| 119 | T | | | | | | | | | C | | | | | | | | | 1 |
| 156 | C | | | | | | | | | | | | | | : | | | | 1 |
| 174 | G | | | | | | | | | | | : | | | | | | | 1 |
| 211 | A | | | | | | | | | | | | : | | | | | | 1 |
| 231 | T | | | | | | | | | | C | | | | | | | | 1 |
| 262 | A | | | : | | | | | | | | | | | | | | | 1 |
| 284 | A | | | | | | | | | | G | | | | | | | | 1 |
| 297 | T | | | | | | | | | | | | | C | | | | | 1 |
| 334 | C | T | | | | | | | | | | | | | | | | | 1 |
| 377 | G | | | | | | | | | | | | | | | N | | | 1 |
| 378 | T | | | | | | | | | | | | | | | N | | | 1 |
| 396 | T | | | : | | | | | | | | | | | | | | | 1 |
| 467 | A | | | : | | | | | | | | | | | | | | | 1 |
| 476 | T | | | | | | | | | | | | | | | | | C | 1 |
| 481 | C | | | : | | | | | | | | | | | | | | | 1 |
| 502 | T | | | | | | | | | | | | | | | : | | | 1 |
| 532 | T | | | | : | | | | | | | | | | | | | | 1 |
| 534 | T | | | | | | C | | | | | | | | | | | | 1 |
| 544 | A | | | | | | | | | | | : | | | | | | | 1 |
| 575 | A | | | | | | | | | | | | | : | | | | | 1 |
| 607 | A | | | | | | | | | | | : | | | | | | | 1 |
| 654 | T | | | | | | | | | | | | | | | | C | | 1 |
| 678 | T | | | | | | | : | | | | | | | | | | | 1 |
| 679 | T | | | | | | | : | | | | | | | | | | | 1 |
| 680 | A | | | : | | | | | | | | | | | | | | | 1 |
| 757 | G | | | | | | | | | | | A | | | | | | | 1 |
| 762 | T | | : | | | | | | | | | | | | | | | | 1 |
| 954 | G | | | | | | | | T | | | | | | | | | | 1 |
| 965 | T | | : | | | | | | | | | | | | | | | | 1 |
| 1,016 | T | | | | | | | | | | | | | | | : | | | 1 |
| 1,018 | G | | | | T | | | | | | | | | | | | | | 1 |
| 1,042 | T | | | | : | | C | | | | | | | | | | | | 2 |
| 1,048 | G | | A | | | | | | | | | | | | | | | | 1 |
| 1,056 | G | | | | | : | | | | | | | | | | | | | 1 |
| 1,057 | T | | | | | : | | | | | | | | | | | | | 1 |
| 1,058 | A | | | | | : | | | | | | | | | | | | | 1 |
| 1,059 | T | | | | | : | | | | | | | | | | | | | 1 |
| 1,060 | T | | | | | : | | | | | | | | | | | | | 1 |
| 1,065 | T | | C | | | | | | | | | | | | | | | | 1 |
| 1,141 | C | | | | | | | | | | | : | | | | | | | 1 |
| 1,187 | T | | | | | | | | | | | C | | | | | | | 1 |
| 1,205 | A | : | | | | | | | | | | | | | | | | | 1 |
| 1,211 | T | : | | | | | | | | | | | | | | | | | 1 |
| 1,226 | A | | | | | | | | | | | | | | | | | G | 1 |
| 1,234 | T | : | | | | | | | | | | | | | | | | | 1 |
| 1,261 | C | | | A | | | | | | | | | | | | | | | 1 |
| 1,288 | C | | | | | | | | | | | | | | | | | N | 1 |
| 1,317 | A | | | | | | | | : | | | | | | | | | | 1 |
| 1,372 | A | | | | | | | | | | | | | | | | G | | 1 |
| 1,524 | T | | | | | | | | | | G | | | | | | | | 1 |
| 1,549 | A | | | | | | | | | | | | | | | T | | | 1 |
| 1,579 | G | | | | | | | | | | | : | | | | | | | 1 |
| 1,619 | T | | | | | | | | | | | | | | | | C | | 1 |
| 1,682 | C | | | | T | | | | | | | | | | | | | | 1 |
| 1,695 | A | | | | | | | | : | | | | | | | | | | 1 |
| 1,706 | C | | : | | | | | | | | | | | | | | | | 1 |
| 1,707 | T | | : | | | | | | | | | | | | | | | | 1 |
| 1,708 | G | | : | | | | | | | | | | | | | | | | 1 |
| 1,709 | A | | : | | | | | | | | | | | | | | | | 1 |
| 1,710 | G | | : | | | | | | | | | | | | | | | | 1 |
| 1,711 | C | | : | | | | | | | | | | | | | | | | 1 |
| 1,712 | A | | : | | | | | | | | | | | | | | | | 1 |
| 1,714 | T | | : | | | | | | | | | | | | | | | | 1 |
| 1,728 | C | | | | | | | | | | | | A | | | | | | 1 |

| | | | | | | | | | | | | | | | | | | |
|---|---|---|---|---|---|---|---|---|---|---|---|---|---|---|---|---|---|---|
| Total | 4 | 12 | 6 | 4 | 15 | 4 | 2 | 3 | 2 | 4 | 8 | 2 | 3 | 1 | 6 | 3 | 3 | 82 |

TABLE 4
Mit construction - Summary of errors from the sequencing of clones
(made by the smPCR procedure) of the 1.8 Kb mitochondrial fragment before error
correction

| Reference | | 1 | 10 | 11 | 12 | 13 | 14 | 15 | 16 | 17 | 18 | 19 | 2 | 20 | 21 | 22 | 25 |
|---|---|---|---|---|---|---|---|---|---|---|---|---|---|---|---|---|---|
| 2 | G | | | | | | | | | | | | | | | | |
| 29 | A | G | | | | | | | | | | | | | | | |
| 30 | C | | | | | | | | | | | : | | | | | |
| 32 | A | | | | | | | : | | | | | | | | | |
| 61 | A | | | | | | | | | | | | | | | | |
| 72 | A | | | | | : | | | | | | | | | | | |
| 74.1 | : | | | | | | | | | | | | | | | | |
| 112 | A | | | | | | | | | | | | | | | | |
| 129 | T | | | | | | | | | | | | | | | | |
| 136 | A | | | | | | | | | | N | | | | | | |
| 137 | T | | | | | | | | | | N | | | | | | |
| 138 | T | | | | | | | | | | N | | | | | | |
| 139 | A | | | | | | | | | | | | | | | | |
| 154 | T | | | | | | | | | | | | | | | | |
| 184 | A | | | | | | | | | | | | | | : | | |
| 198 | T | | | | | | | | | | | | | | | | |
| 206 | G | | | | | | | | | | | | | | : | | |
| 244 | C | | | | | | | | | | | | | | | | |
| 247 | T | | | | | | | | | C | | | | | | | |
| 258 | T | | | | | | | | | | | C | | | | | |
| 262 | A | | G | | | | | | | | | | | | | | |
| 264 | C | | | | | | | | | | | | | | | | |
| 276 | T | | | | | | | | | | | | | | | | |
| 282 | C | | | | | | | | | | | | | | | | |
| 287 | G | | | | A | | | | | | | | | | | | |
| 288 | T | | | | | | | | | | | | | | | | |
| 292 | T | | | | | | | | | | | | | | C | | |
| 319 | A | | | | | | | | | | | | G | | | | |
| 330 | A | | | | | | | | | | | | | | | | |
| 337 | A | | | | | | | | | | | | | | | | |
| 350 | T | | | | | | | | | | | | | | | | |
| 355 | A | | | | | | | | | | | | | | | | |
| 368 | G | | | | | | | | | | | | | : | | : | |
| 381 | T | | | | | | | | | | | | | | | | |
| 393 | C | | | | | | | | | | | | | | | | : |
| 453 | T | | | | | | | | C | | | | | | | | |
| 484 | A | | | | | : | | | | | | | | | | | |
| 490 | T | | | | | | | | | | | | | | | | |
| 491 | A | | G | | | | | | | | | | | | | | |
| 498 | T | | | | | | | | : | | | | | | | | |
| 499 | A | | | | | | | | | | | | | | | | |
| 520 | A | | | | | | | | | | | | | : | | | |
| 521 | T | | | | | | | | | | | | | | | | |
| 537 | T | | | | | | | | : | | | | | | | | |
| 538 | T | | | | | | | | : | | | | | | | | |
| 539 | T | | | | | | | | : | | | | | | | | |
| 572 | T | | | | | | | | | | | | | | | | |
| 579 | C | | | | | | | | | | | | | | | | |
| 583 | T | | | | | | | | | | | | | | | | |
| 585 | A | | | | | | | | | C | | | | | | | |
| 589 | A | | | | | | | | | | | | | | | | |
| 620 | A | | | | | | | | | | | | | | G | | |
| 627 | T | | | | | C | | | | | | | | | | | |
| 634 | T | | | | | | | | : | | | | | | | | |
| 635 | T | | | | | | | | : | | | | | | | | |
| 640 | T | | | | | | | | | | | | | : | | | |
| 656.1 | : | | | | | | | | | | | | | | | | |
| 673 | G | | | | | | | | | | | | | | | | |
| 686 | A | | | | | | | | | | | | | | | | |
| 726 | G | | | | | | | A | | | | | | | | | |
| 727 | T | | | | | | | | | | | | | | | | |
| 733 | A | | | | | | | | | | | | : | | | | G |
| 751 | G | | | | | | | | | | | | | | | | |
| 784 | C | : | | | | | | | | | | | | | | | |
| 785 | A | : | | | | | | | | | | | | | | | |
| 787 | G | C | | | | | | | | | | | | | | | |
| 789 | G | : | | | | | | | | | | | | | | | |
| 790 | C | : | | | | | | | | | | | | | | | |
| 792 | T | : | | | | | | | | | | | | | | | |
| 802 | T | | | | | | | | | | | | | | | C | |
| 810 | A | | | | | | | | | | | | | | | | |
| 829 | G | | | | | | | | | | | | | | | | |
| 859 | C | | | | | | | | | | A | | | | | | |
| 874 | C | | | | | | | | | | | T | | | | | |
| 877 | G | | T | | | | | | | | | | | | | | |

| | | | | | | | | | | | | | | | | |
|---|---|---|---|---|---|---|---|---|---|---|---|---|---|---|---|---|
| Total | 7 | 3 | 0 | 1 | 3 | 0 | 2 | 7 | 2 | 4 | 4 | 1 | 3 | 4 | 2 | 1 |

| Reference | | 26 | 27 | 28 | 29 | 3 | 30 | 33 | 34 | 4 | 5 | 6 | 7 | 9 | Total |
|---|---|---|---|---|---|---|---|---|---|---|---|---|---|---|---|
| 2 | G | | | | | | | | | | | | : | | 1 |
| 29 | A | | | | | | | | | | | | | | 1 |
| 30 | C | | | | | | | | | | | | | | 1 |
| 32 | A | | | | | | | | | | | | | | 1 |
| 61 | A | | | | G | | | | | | | | | | 1 |
| 72 | A | | | | | | | | | | | | | | 1 |
| 74.1 | : | | | T | | | | | | | | | | | 1 |
| 112 | A | | T | | | | | | | | | | | | 1 |
| 129 | T | | C | | | | | | | | | | | | 1 |
| 136 | A | | | | | | | | | | | | | | 1 |
| 137 | T | | | | | | | | | | | | | | 1 |
| 138 | T | | | | | | | | | | | | | | 1 |
| 139 | A | | | | | | | G | | | | | | | 1 |
| 154 | T | | | | | | | : | | | : | | | | 2 |
| 184 | A | | | | | | | | | | | | | | 1 |
| 198 | T | | | | | | | C | | | | | | | 1 |
| 206 | G | | | | | | | | | | | | | | 1 |
| 244 | C | | | | | | | | | | : | | | | 1 |
| 247 | T | | | | | | | | | | | | | | 1 |
| 258 | T | | | | | | | | | | | | | | 1 |
| 262 | A | | | | | | | | | | | | | | 1 |
| 264 | C | | | : | | | | | | | | | | | 1 |
| 276 | T | | | | | | | | | | | C | | | 1 |
| 282 | C | | | | | | | | | | A | | | | 1 |
| 287 | G | | | | | | | | | | | | | | 1 |
| 288 | T | | | | | | | | | | | | | G | 1 |
| 292 | T | | | | | | | | | | | | | | 1 |
| 319 | A | | | | | | | | | | | | | | 1 |
| 330 | A | | | | G | | | | | | | | | | 1 |
| 337 | A | | | | | | | G | | | | | | | 1 |
| 350 | T | | | C | | | | | | | | | | | 1 |
| 355 | A | | G | | | | | | | | | | | | 1 |
| 368 | G | | | | | | | | | | | | | | 2 |
| 381 | T | | | | | : | | | | | | | | | 1 |
| 393 | C | | | | | | | | | | | | | | 1 |
| 453 | T | | | | | | | | | | | | | | 1 |
| 484 | A | | | | | | | | | | | | | | 1 |
| 490 | T | | | | | C | | | | | | | | | 1 |
| 491 | A | | | | | | | | | | | | | | 1 |
| 498 | T | | | | | | | | | | | | | | 1 |
| 499 | A | G | | | | | | | | | | | | | 1 |
| 520 | A | | | | | | | | | | | | | | 1 |
| 521 | T | | | | | | | | | | | C | | | 1 |
| 537 | T | | | | | | | | | | | | | | 1 |
| 538 | T | | | | | | | | | | | | | | 1 |
| 539 | T | | | | | | | | | | | | | : | 2 |
| 572 | T | | | | | | | | C | | | | | | 1 |
| 579 | C | | T | | | | | | | | | | | | 1 |
| 583 | T | | | | | | | | | | | | | C | 1 |
| 585 | A | | | | | | | | | | | | | | 1 |
| 589 | A | | G | | | | | | | | | | | | 1 |
| 620 | A | | | | | | | | | | | | | | 1 |
| 627 | T | | | | | | | | | | | | | | 1 |
| 634 | T | | | | | | | | | | | | | | 1 |
| 635 | T | | | | | | | | | | | | | | 1 |
| 640 | T | | | | | | | | | | | | | | 1 |
| 656.1 | : | | | T | | | | | | | | | | | 1 |
| 673 | G | | | : | | | | | | | : | | | | 2 |
| 686 | A | | | | | | | | | | | | | G | 1 |
| 726 | G | | | | | | | | | | | | | | 1 |
| 727 | T | | | C | | | | | | | | | | | 1 |
| 733 | A | | | | | | | | | | | | | | 2 |
| 751 | G | | | | | T | | | | | | | | | 1 |
| 784 | C | | | | | | | | | | | | | | 1 |
| 785 | A | | | | | | | | | | | | | | 1 |
| 787 | G | | | | | | | | | | | | | | 1 |
| 789 | G | | | | | | | | | | | | | | 1 |
| 790 | C | | | | | | | | | | | | | | 1 |
| 792 | T | | | | | | | | | | | | | | 1 |
| 802 | T | | | | | | | | | | | | | | 1 |
| 810 | A | | | | | | | | | | | G | | | 1 |
| 829 | G | | | | | | T | | | | | | | | 1 |
| 859 | C | | | | | | | | | | | | | | 1 |
| 874 | C | | | | | | | | | | | | | | 1 |
| 877 | G | | | | | | | | | | | | | | 1 |

| | | | | | | | | | | | | | | |
|---|---|---|---|---|---|---|---|---|---|---|---|---|---|---|
| Total | 2 | 5 | 6 | 2 | 3 | 1 | 4 | 1 | 0 | 4 | 3 | 1 | 4 | 80 |

TABLE 5
Mit reconstruction - Summary of errors from the sequencing of clones
(made by in vivo cloning) of the 1.8 Kb mitochondrial fragment after error
correction (using in vivo cloning)

| Reference | | 001 | 002 | 003 | 004 | 005 | 006 | 007 | 008 | 010 | 012 | Total |
|---|---|---|---|---|---|---|---|---|---|---|---|---|
| 82 | T | C | | | | | | | | | | 1 |
| 953 | G | | | | | | A | | | | | 1 |
| 1,089 | T | | | | | | | A | | | | 1 |
| 1,321 | T | : | | | | | | | | | | 1 |
| 1,322 | T | : | | | | | | | | | | 1 |
| 1,401 | G | | | | | | | | | | A | 1 |
| 1,662 | C | | | | | | | | | | A | 1 |
| 1,670 | C | | | A | | | | | | | | 1 |

| | | | | | | | | | | | |
|---|---|---|---|---|---|---|---|---|---|---|---|
| Total | 3 | 0 | 1 | 0 | 0 | 1 | 1 | 0 | 0 | 2 | 8 |

TABLE 6
Mit reconstruction - Summary of errors from the sequencing of clones
(made by in vivo cloning) of the 1.8 Kb mitochondrial fragment after error
correction (using the smPCR procedure)

| Reference | | 001 | 002 | 003 | 004 | 005 | 007 | 009 | 010 | 012 | Total |
|---|---|---|---|---|---|---|---|---|---|---|---|
| 102 | T | | C | | | | | | | | 1 |
| 125 | C | | | | T | | | | | | 1 |
| 176 | G | | | | | | | | | T | 1 |
| 293 | G | A | | | | | | | | | 1 |
| 394 | T | | | | | | | A | | | 1 |
| 596 | A | | | G | | | | | | | 1 |
| 722 | T | | C | | | | | | | | 1 |
| 764 | T | | | | | | | | | C | 1 |
| 864 | T | | | : | | | | | | | 1 |
| 926 | A | | | | | | | | G | | 1 |
| 952 | G | | | | | | | | T | | 1 |
| 1,012 | G | | | | | | | | | T | 1 |
| 1,214 | T | | | | | C | | | | | 1 |
| 1,269 | C | | A | | | | | | | | 1 |

| | | | | | | | | | | |
|---|---|---|---|---|---|---|---|---|---|---|
| Total | 1 | 3 | 2 | 1 | 1 | 0 | 1 | 2 | 3 | 14 |

### REFERENCES

1. Bang, D. and Church, G. M. (2008) Gene synthesis by circular assembly amplification. Nat Methods, 5, 37-39.
2. Carr, P. A., Park, J. S., Lee, Y. J., Yu, T., Zhang, S, and Jacobson, J. M. (2004) Protein-mediated error correction for de novo DNA synthesis. Nucleic Acids Res, 32, e162.
3. Kodumal, S. J., Patel, K. G., Reid, R., Menzella, H. G., Welch, M. and Santi, D. V. (2004) Total synthesis of long DNA sequences: synthesis of a contiguous 32-kb polyketide synthase gene cluster. Proc Natl Acad Sci USA, 101, 15573-15578.
4. Tian, J., Gong, H., Sheng, N., Zhou, X., Gulari, E., Gao, X. and Church, G. (2004) Accurate multiplex gene synthesis from programmable DNA microchips. Nature, 432, 1050-1054.
5. Linshiz, G., Yehezkel, T. B., Kaplan, S., Gronau, I., Ravid, S., Adar, R. and Shapiro, E. (2008) Recursive construction of perfect DNA molecules from imperfect oligonucleotides. Mol Syst Biol, 4, 191.
6. Xiong, A. S., Yao, Q. H., Peng, R. H., Duan, H., Li, X., Fan, H. Q., Cheng, Z. M. and Li, Y. (2006) PCR-based accurate synthesis of long DNA sequences. Nat Protoc, 1, 791-797.
7. Xiong, A. S., Yao, Q. H., Peng, R. H., Li, X., Fan, H. Q., Cheng, Z. M. and Li, Y. (2004) A simple, rapid, high-fidelity and cost-effective PCR-based two-step DNA synthesis method for long gene sequences. Nucleic Acids Res, 32, e98.
8. Saiki, R. K., Gelfand, D. H., Stoffel, S., Scharf, S. J., Higuchi, R., Horn, G. T., Mullis, K. B. and Erlich, H. A. (1988) Primer-directed enzymatic amplification of DNA with a thermostable DNA polymerase. Science, 239, 487-491.
9. Ohuchi, S., Nakano, H. and Yamane, T. (1998) In vitro method for the generation of protein libraries using PCR amplification of a single DNA molecule and coupled transcription/translation. Nucleic Acids Res, 26, 4339-4346.
10. Nakano, M., Komatsu, J., Kurita, H., Yasuda, H., Katsura, S, and Mizuno, A. (2005) Adaptor polymerase chain reaction for single molecule amplification. J Biosci Bioeng, 100, 216-218.
11. Kraytsberg, Y. and Khrapko, K. (2005) Single-molecule PCR: an artifact-free PCR approach for the analysis of somatic mutations. Expert Rev Mol Diagn, 5, 809-815.
12. Lukyanov, K. A., Matz, M. V., Bogdanova, E. A., Gurskaya, N. G. and Lukyanov, S. A. (1996) Molecule by molecule PCR amplification of complex DNA mixtures for direct sequencing: an approach to in vitro cloning. Nucleic Acids Res, 24, 2194-2195.
13. Margulies, M., Egholm, M., Altman, W. E., Attiya, S., Bader, J. S., Bemben, L. A., Berka, J., Braverman, M. S., Chen, Y. J., Chen, Z. et al. (2005) Genome sequencing in microfabricated high-density picolitre reactors. Nature, 437, 376-380.
14. Shendure, J., Porreca, G. J., Reppas, N. B., Lin, X., McCutcheon, J. P., Rosenbaum, A. M., Wang, M. D., Zhang, K., Mitra, R. D. and Church, G. M. (2005) Accurate multiplex polony sequencing of an evolved bacterial genome. Science, 309, 1728-1732.
15. Hecker, K. H. and Rill, R. L. (1998) Error analysis of chemically synthesized polynucleotides. Biotechniques, 24, 256-260.
16. Nakano, H., Kobayashi, K., Ohuchi, S., Sekiguchi, S, and Yamane, T. (2000) Single-step single-molecule PCR of DNA with a homo-priming sequence using a single primer and hot-startable DNA polymerase. J Biosci Bioeng, 90, 456-458.
17. Tindall; K. R. and Kunkel, T. A. (1988) Fidelity of DNA synthesis by the Thermus aquaticus DNA polymerase. Biochemistry, 27, 6008-6013.
18. Cline, J., Braman, J. C. and Hogrefe, H. H. (1996) PCR fidelity of pfu DNA polymerase and other thermostable DNA polymerases. Nucleic Acids Res, 24, 3546-3551.
19. Hutchison, C. A., 3rd, Smith, H. O., Pfannkoch, C. and Venter, J. C. (2005) Cell-free cloning using phi29 DNA polymerase. Proc Natl Acad Sci USA, 102, 17332-17336.
20. Esteban, J. A., Salas, M. and Blanco, L. (1993) Fidelity of phi 29 DNA polymerase. Comparison between protein-primed initiation and DNA polymerization. J Biol Chem, 268, 2719-2726.

SEQUENCE LISTING

GFP Intermediate and Final Fragments
>GFP_1_16
SEQ ID NO: 1
GGATCCACCGGTCGCCACCATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGGGCGAGGGCGATGCCACCTA
CGGCAAGCTGACCCTGAAGTTCATCTGCACCACCGGCAAGCTGCCCGTGCCCTGGCCCACCCTCGTGAC
CACCCTGACCTACGGCGTGCAGTGCTTCAGCCGCTACCCCGACCACATGAAGCAGCACGACTTCTTCAA
GTCCGCCATGCCCGAAGGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTGAAGGGCATCGACTTCAA
GGAGGACGGCAACATCCTGGGGCACAAGCTGGAGTACAACTACAACAGCCACAACGTCTATATCATGGC
CGACAAGCAGAAGAACGGCATCAAGGTGAACTTCAAGATCCGCCACAACATCGAGGACGGCAGCGTGCA
GCTCGCCGACCACTACCAGCAGAACACCCCCATCGGCGACGGCCCCGTGCTGCTGCCCGACAACCACTA
CCTGAGCACCCAGTCCGCCCTGAGCAAAGACCCCAACGAGAAGCGCGATCACATGGTCCTGCTGGAGTT
CGTGACCGCCGCCGGGATCACTCTCGGCATGGACGAGCTGTACAAGTAAAGCGGCCGCGACTCTAGATC
ATAATCAGC
>GFP_1_8
SEQ ID NO: 2
GGATCCACCGGTCGCCACCATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGGGCGAGGGCGATGCCACCTA
CGGCAAGCTGACCCTGAAGTTCATCTGCACCACCGGCAAGCTGCCCGTGCCCTGGCCCACCCTCGTGAC
CACCCTGACCTACGGCGTGCAGTGCTTCAGCCGCTACCCCGACCACATGAAGCAGCACGACTTCTTCAA
GTCCGCCATGCCCGAAGGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTGAAGGGCATCGACTTCAA
GGAGGACGGCAACATCCTGGGGCACA
>GFP_9_16
SEQ ID NO: 3
TCAAGGAGGACGGCAACATCCTGGGGCACAAGCTGGAGTACAACTACAACAGCCACAACGTCTATATCA
TGGCCGACAAGCAGAAGAACGGCATCAAGGTGAACTTCAAGATCCGCCACAACATCGAGGACGGCAGCG
TGCAGCTCGCCGACCACTACCAGCAGAACACCCCCATCGGCGACGGCCCCGTGCTGCTGCCCGACAACC
ACTACCTGAGCACCCAGTCCGCCCTGAGCAAAGACCCCAACGAGAAGCGCGATCACATGGTCCTGCTGG
AGTTCGTGACCGCCGCCGGGATCACTCTCGGCATGGACGAGCTGTACAAGTAAAGCGGCCGCGACTCTA
GATCATAATCAGC
>GFP_1_4
SEQ ID NO: 4
GGATCCACCGGTCGCCACCATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGGGCGAGGGCGATGCCACCTA
CGGCAAGCTGACCCTGAAGTTCATCTGCACCACCGGCAAGCTGCCCGTGCCCTGGCCCACCCTCGTGAC
CACCCTGACCTACGGCGTGCAGTGCTTCAGCCGCT
>GFP_5_8
SEQ ID NO: 5
ACGGCGTGCAGTGCTTCAGCCGCTACCCCGACCACATGAAGCAGCACGACTTCTTCAAGTCCGCCATGC
CCGAAGGCTACGTCCAGGAGCGCACCATCTTCTTCAAGGACGACGGCAACTACAAGACCCGCGCCGAGG
TGAAGTTCGAGGGCGACACCCTGGTGAACCGCATCGAGCTGAAGGGCATCGACTTCAAGGAGGACGGCA
ACATCCTGGGGCACA
>GFP_9_12
SEQ ID NO: 6
TCAAGGAGGACGGCAACATCCTGGGGCACAAGCTGGAGTACAACTACAACAGCCACAACGTCTATATCA
TGGCCGACAAGCAGAAGAACGGCATCAAGGTGAACTTCAAGATCCGCCACAACATCGAGGACGGCAGCG
TGCAGCTCGCCGACCACTACCAGCAGAACACCCCCATCGGCG
>GFP_13_16
SEQ ID NO: 7
ACCAGCAGAACACCCCCATCGGCGACGGCCCCGTGCTGCTGCCCGACAACCACTACCTGAGCACCCAGT
CCGCCCTGAGCAAAGACCCCAACGAGAAGCGCGATCACATGGTCCTGCTGGAGTTCGTGACCGCCGCCG
GGATCACTCTCGGCATGGACGAGCTGTACAAGTAAAGCGGCCGCGACTCTAGATCATAATCAGC
>GFP_1_2
SEQ ID NO: 8
GGATCCACCGGTCGCCACCATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGGGCGAGGGCGATGCC
>GFP_3_4
SEQ ID NO: 9
GTCCGGCGAGGGCGAGGGCGATGCCACCTACGGCAAGCTGACCCTGAAGTTCATCTGCACCACCGGCAA
GCTGCCCGTGCCCTGGCCCACCCTCGTGACCACCCTGACCTACGGCGTGCAGTGCTTCAGCCGCT
>GFP_5_6
SEQ ID NO: 10
ACGGCGTGCAGTGCTTCAGCCGCTACCCCGACCACATGAAGCAGCACGACTTCTTCAAGTCCGCCATGC
CCGAAGGCTACGTCCAGGAGCGCACCATCTTCTTCAAGGACGACGGCAACTACAAGACCCGCGCCG
>GFP_7_8
SEQ ID NO: 11
CAAGGACGACGGCAACTACAAGACCCGCGCCGAGGTGAAGTTCGAGGGCGACACCCTGGTGAACCGCAT
CGAGCTGAAGGGCATCGACTTCAAGGAGGACGGCAACATCCTGGGGCACA
>GFP_9_10
SEQ ID NO: 12
TCAAGGAGGACGGCAACATCCTGGGGCACAAGCTGGAGTACAACTACAACAGCCACAACGTCTATATCA
TGGCCGACAAGCAGAAGAACGGCATC
>GFP_11_12
SEQ ID NO: 13
GGCCGACAAGCAGAAGAACGGCATCAAGGTGAACTTCAAGATCCGCCACAACATCGAGGACGGCAGCGT
GCAGCTCGCCGACCACTACCAGCAGAACACCCCCATCGGCG
>GFP_13_14
SEQ ID NO: 14
ACCAGCAGAACACCCCCATCGGCGACGGCCCCGTGCTGCTGCCCGACAACCACTACCTGAGCACCCAGT
CCGCCCTGAGCAAAGACCCCAACGAGAAGCGCG
>GFP_15_16
SEQ ID NO: 15
GAGCAAAGACCCCAACGAGAAGCGCGATCACATGGTCCTGCTGGAGTTCGTGACCGCCGCCGGGATCAC
TCTCGGCATGGACGAGCTGTACAAGTAAAGCGGCCGCGACTCTAGATCATAATCAGC
GFP Oligos
>Oli_1
SEQ ID NO: 16
GGATCCACCGGTCGCCACCATGGTGAGCAAGGGCGAGGAGCTGTTCACCGGGGTGGTGCCCATCCTGGT
CGAGCTGG
>Oli_2
SEQ ID NO: 17
GGCATCGCCCTCGCCCTCGCCGGACACGCTGAACTTGTGGCCGTTTACGTCGCCGTCCAGCTCGACCAG
GATGGGCACC
>Oli_3
SEQ ID NO: 18
GTCCGGCGAGGGCGAGGGCGATGCCACCTACGGCAAGCTGACCCTGAAGTTCATCTGCACCACCGGCA
>Oli_4
SEQ ID NO: 19
AGCGGCTGAAGCACTGCACGCCGTAGGTCAGGGTGGTCACGAGGGTGGGCCAGGGCACGGGCAGCTTGC
CGGTGGTGCAGATGA
>Oli_5
SEQ ID NO: 20
ACGGCGTGCAGTGCTTCAGCCGCTACCCCGACCACATGAAGCAGCACGACTTCTTCAAGTCCGCCATGC
CCGAAGGCTACGTCC
>Oli_6
SEQ ID NO: 21
CGGCGCGGGTCTTGTAGTTGCCGTCGTCCTTGAAGAAGATGGTGCGCTCCTGGACGTAGCCTTCGGGCA
TGGCGGACTTG
>Oli_7
SEQ ID NO: 22
CAAGGACGACGGCAACTACAAGACCCGCGCCGAGGTGAAGTTCGAGGGCGACACCCTGGTGAACCGC
>Oli_8
SEQ ID NO: 23
TGTGCCCCAGGATGTTGCCGTCCTCCTTGAAGTCGATGCCCTTCAGCTCGATGCGGTTCACCAGGGTGT
CGCCCTCGAACTTC
>Oli_9
SEQ ID NO: 24
TCAAGGAGGACGGCAACATCCTGGGGCACAAGCTGGAGTACAACTACAACAGCCACAACGTCT
>Oli_10
SEQ ID NO: 25
GATGCCGTTCTTCTGCTTGTCGGCCATGATATAGACGTTGTGGCTGTTGTAGTTGTACTCC
>Oli_11
SEQ ID NO: 26
GGCCGACAAGCAGAAGAACGGCATCAAGGTGAACTTCAAGATCCGCCACAACATCGAGGACGGC
>Oli_12
SEQ ID NO: 27
CGCCGATGGGGGTGTTCTGCTGGTAGTGGTCGGCGAGCTGCACGCTGCCGTCCTCGATGTTGTGGCGGA
TCTTG
>Oli_13
SEQ ID NO: 28
ACCAGCAGAACACCCCCATCGGCGACGGCCCCGTGCTGCTGCCCGACAACCACTACCTGAGC
>Oli_14
SEQ ID NO: 29
CGCGCTTCTCGTTGGGGTCTTTGCTCAGGGCGGACTGGGTGCTCAGGTAGTGGTTGTCGGGC
>Oli_15
SEQ ID NO: 30
GAGCAAAGACCCCAACGAGAAGCGCGATCACATGGTCCTGCTGGAGTTCGTGACCGCCGCCGGGATCAC
TCTCGGCATGG
>Oli_16
SEQ ID NO: 31
GCTGATTATGATCTAGAGTCGCGGCCGCTTTACTTGTACAGCTCGTCCATGCCGAGAGTGATCCCGGCG
GCGGTC
GFP Primers
>Prm_1F
SEQ ID NO: 32
GGATCCACCGGTCGCCA
>Prm_3F
SEQ ID NO: 33
GTCCGGCGAGGGCGA
>Prm_5F
SEQ ID NO: 34
ACGGCGTGCAGTGCTTC
>Prm_7F
SEQ ID NO: 35
CAAGGACGACGGCAACTACAA
>Prm_9F
SEQ ID NO: 36
TCAAGGAGGACGGCAACAT
>Prm_11F
SEQ ID NO: 37
GGCCGACAAGCAGAAGAAC
>Prm_13F
SEQ ID NO: 38
ACCAGCAGAACACCCCCAT
>Prm_15F
SEQ ID NO: 39
GAGCAAAGACCCCAACGAGA
>Prm_2R
SEQ ID NO: 40
GGCATCGCCCTCGCC
>Prm_4R
SEQ ID NO: 41
AGCGGCTGAAGCACTGCAC
>Prm_6R
SEQ ID NO: 42
CGGCGCGGGTCTTGTA
>Prm_8R
SEQ ID NO: 43
TGTGCCCCAGGATGTTG
>Prm_10R
SEQ ID NO: 44
GATGCCGTTCTTCTGCTTGTC
>Prm_12R
SEQ ID NO: 45
CGCCGATGGGGGTGT
>Prm_14R
SEQ ID NO: 46
CGCGCTTCTCGTTGGG
>Prm_16R
SEQ ID NO: 47
GGCTGATTATGATCTAGAGTCGCGG

All Oligos, Primers, Intermediates and Full Length Sequences from the Construction of the 1.8 Kb Mitochondrial DNA Fragment

Primers:
> Primer_1
SEQ ID NO: 48
AGTAGATACAAGAGCATATTTTACTTC
> Primer_2
SEQ ID NO: 49
AAAACATAATTATAACCTTACGGTCTG
> Primer_3
SEQ ID NO: 50
ATAGATATTAAGAATATCATTAATCCAGATATCCATGATAAAGGTAAAT
> Primer_4
SEQ ID NO: 51
ACCTTTATCATGGATATCTGGATTAATGATATTCTTAATATCTATTGTT
ACAGC
> Primer_5
SEQ ID NO: 52
GCGTCTGGATAATCAGGAATACGTCTAGGCA
> Primer_6
SEQ ID NO: 53
CTAGACGTATTCCTGATTATCCAGACGCTTTAAATGGTTG
> Primer_7
SEQ ID NO: 54
AATTACATAATATGTATCATGTAAAGCTATGTCAATAGCAG
> Primer_8
SEQ ID NO: 55
CTGCTATTGACATAGCTTTACATGATACATATTATGTAATTGCTCATTT
CCACTTT
> Primer_11
SEQ ID NO: 56
AGTAGATACAAGAGCATATTTTACTTCTACAACTATTTTAATATCTATT
CCAACTGGTACAAAAGTAT
> Primer_12
SEQ ID NO: 57
ATGTATCATGTAAAGCTATGTCAATAGCAGCATTACCTAATATTACTCC
AGTAGTACCACCAAAAGTAAA
> Primer_15
SEQ ID NO: 58
ATTATGTAATTGCTCATTTCCACTTTGTTCTATCAATTGGAGCAATTAT
TGCATTATTTACAACAG
> Primer_16
SEQ ID NO: 59
AATCAGGAATACGTCTAGGCATTACATTAAATCCAAGGAAATGCATAGG
TAAAAATGTTAAAACTACA
> Primer_17
SEQ ID NO: 60
GTTGCTAATAATACACCTGTTAAAATTTGTATAAAAAATACTATTCCTA
AAAGAAATCC
> Primer_18
SEQ ID NO: 61
AGGAATAGTATTTTTTATACAAATTTTAACAGGTGTATTATTAGCAACT
> Primer_21
SEQ ID NO: 62
ATCCAGACGCTTTAAATGGTTGGAATATGATTTGCTCTATCGGATCAAC
TATGACTTTATTTGGTTTATTAATTTTTA
> Primer_22
SEQ ID NO: 63
TGTATAAAAAATACTATTCCTAAAAGAAATCCATAATTCCATAAGAAAT
TAATATTTAGTGGACATGGATAATG
> Primer_25
SEQ ID NO: 64
AATTTTAACAGGTGTATTATTAGCAACTTGTTATACTCCAGAAATATCT
TATGCATATTATAGTGTACAAC
> Primer_26
SEQ ID NO: 65
TCCAGATATCCATGATAAAGGTAAATATGAATATGAATAATTTAATCCT
CTTAAAATATGTAAGTAAGTTAA
> Primer_27
SEQ ID NO: 66
AAGAAATACCATTCTGGAACAATATGTAAAGGTGTAGCATATCTATCTA
CTGTAATTG
> Primer_28
SEQ ID NO: 67
GCTACACCTTTACATATTGTTCCAGAATGGTATTTCTTACCT
> Primer_29
SEQ ID NO: 68
ATGTGTATAAATACGATACATAAAGCTATAAATGGAA
> Primer_30
SEQ ID NO: 69
CATTTTACTTTTCCATTTATAGCTTTATGTATCGTATTTATACACATAT
TCTTCTTACATCTACAAG
> Primer_33
SEQ ID NO: 70
TTAATGATATTCTTAATATCTATTGTTACAGCTTTTATGGGTTATGTAT
TACCTTGGGGTCAAATGA
> Primer_34
SEQ ID NO: 71
ATACGATACATAAAGCTATAAATGGAAAAGTAAAATGTAATACAAAGAA
TCTTTTTAAAGTTGGGTCAC
> Primer_37
SEQ ID NO: 72
TTATACACATATTCTTCTTACATCTACAAGGTAGCACTAATCCTTTAGG
GTATGATACAGCTTTAAAAA
> Primer_38
SEQ ID NO: 73
TATGTAAAGGTGTAGCATATCTATCTACTGTAATTGCATTATCTGGATG
TGATAATGGTAATATTCCA
> Primer_39
SEQ ID NO: 74
CATCCAATCCATAATAAAGCATAGAATGAACATATAAACCAAATTGT
> Primer_40
SEQ ID NO: 75
TTGGTTTATATGTTCATTCTATGCTTTATTATGGATTGGATGTCAATTA
CCACAAGA
> Primer_43
SEQ ID NO: 76
TTGTTCCAGAATGGTATTTCTTACCTTTTTATGCAATGTTAAAAACCAT
TCCTAACAAAACTGCTG
> Primer_44
SEQ ID NO: 77
ATAATAAAGCATAGAATGAACATATAAACCAAATTGTAGGAACTGAATA
TTCTCTAGCACCAA
> Primer_47
SEQ ID NO: 78
GGATTGGATGTCAATTACCACAAGATATTTACATTTTATATGGTCGTTT
ATTTATTATATTATTCTTTTTTAGTG
> Primer_48
SEQ ID NO: 79
AAAACATAATTATAACCTTACGGTCTGTATTGTTCCGCTCAATGCTCAG
AAATGTCGTCTT
Oligos
> Oligo_9
SEQ ID NO: 80
TATCTATTCCAACTGGTACAAAAGTATTTAATTGGATATGTACATATAT
GGGTAGTAATTTTGGAATAACACATAGTTC
> Oligo_10
SEQ ID NO: 81
ATTACTCCAGTAGTACCACCAAAAGTAAATGTACATATAAATAATAATG
CTAGAAGAGATGAACTATGTGTTATTCCAAA
> Oligo_13
SEQ ID NO: 82
AGCAATTATTGCATTATTTACAACAGTAAGTGCATTCCAAGAAAATTTC
TTTGGTAAACATTTACGTGAAAACTCAATTA
> Oligo_14
SEQ ID NO: 83
ATGCATAGGTAAAAATGTTAAAACTACACCTACGAAGAATAACATTGAC
CATAATATAATAATTGAGTTTTCACGTAA
> Oligo_19
SEQ ID NO: 84
ATCAACTATGACTTTATTTGGTTTATTAATTTTTAAATAATATATAACT
ATTTTTTGTTTATATGAATTATTATTCTATT
> Oligo_20
SEQ ID NO: 85
AGAAATTAATATTTAGTGGACATGGATAATGAATTAAGTGTGCTTTAGC
TAAATTAATAGAATAATAATTCATATAAACA
> Oligo_23
SEQ ID NO: 86
AGAAATATCTTATGCATATTATAGTGTACAACACATATTAAGAGAATTA
TGGAGTGGATGGTGTTTTAGATATATGCATG
> Oligo_24
SEQ ID NO: 87
ATTTAATCCTCTTAAAATATGTAAGTAAGTTAAAATAAATACAAATGAA
GCACCTGTTGCATGCATATATCTAAAACAC
> Oligo_31
SEQ ID NO: 88
TTATGTATTACCTTGGGGTCAAATGAGTTTCTGGGGTGCTACTGTTATA
ACTAATTTACTTTATTTTATTCCTGGACT
> Oligo_32
SEQ ID NO: 89
ACAAAGAATCTTTTTAAAGTTGGGTCACTTACAAGATATCCACCACAAA
TCCATGAAACAAGTCCAGGAATAAAATAAAG
> Oligo_35
SEQ ID NO: 90
CTTTAGGGTATGATACAGCTTTAAAAATACCCTTCTATCCAAATCTTTT
AAGTCTTGACATTAAAGGATTTAATAATGTA
> Oligo_36
SEQ ID NO: 91
CTGGATGTGATAATGGTAATATTCCAAATAAACTTTGAGCTAAGAATAA
TACTAATACATTATTAAATCCTTTAATGTC
> Oligo_41
SEQ ID NO: 92
AAAAACCATTCCTAACAAAACTGCTGGTTTATTAGTTATGTTAGCATCA
CTACAAATATTATTTCTATTAGCAGAACA
> Oligo_42
SEQ ID NO: 93
AACTGAATATTCTCTAGCACCAAAAGCAAATTTAAATTGGATAAGAGTT
GTTAAATTTCTTTGTTCTGCTAATAGAAATA
> Oligo_45
SEQ ID NO: 94
GGTCGTTTATTTATTATATTATTCTTTTTTAGTGGTTTATTTACACTTG
TTCAATCTAAAAGAACACATTATGATTACAG
> Oligo_46
SEQ ID NO: 95
ATGCTCAGAAATGTCGTCTTATCGCAGCCTTGTAATATTAAATGTTTGC
TTGGGAGCTGTAATCATAATGTGTTCT

### All Intermediates and Full Length:

Node id 1:
SEQ ID NO: 96
AGTAGATACAAGAGCATATTTTACTTCTACAACTATTTTAATATCTATT
CCAACTGGTACAAAAGTATTTAATTGGATATGTACATATATGGGTAGTA
ATTTTGGAATAACACATAGTTCATCTCTTCTAGCATTATTATTTATATG
TACATTTACTTTTGGTGGTACTACTGGAGTAATATTAGGTAATGCTGCT
ATTGACATAGCTTTACATGATACATATTATGTAATTGCTCATTTCCACT
TTGTTCTATCAATTGGAGCAATTATTGCATTATTTACAACAGTAAGTGC
ATTCCAAGAAAATTTCTTTGGTAAACATTTACGTGAAAACTCAATTATT
ATATTATGGTCAATGTTATTCTTCGTAGGTGTAGTTTTAACATTTTTAC
CTATGCATTTCCTTGGATTTAATGTAATGCCTAGACGTATTCCTGATTA
TCCAGACGCTTTAAATGGTTGGAATATGATTTGCTCTATCGGATCAACT
ATGACTTTATTTGGTTTATTAATTTTTAAATAATATATAACTATTTTTT
GTTTATATGAATTATTATTCTATTAATTTAGCTAAAGCACACTTAATTC
ATTATCCATGTCCACTAAATATTAATTTCTTATGGAATTATGGATTTCT
TTTAGGAATAGTATTTTTTATACAAATTTTAACAGGTGTATTATTAGCA
ACTTGTTATACTCCAGAAATATCTTATGCATATTATAGTGTACAACACA
TATTAAGAGAATTATGGAGTGGATGGTGTTTTAGATATATGCATGCAAC
AGGTGCTTCATTTGTATTTATTTTAACTTACTTACATATTTTAAGAGGA
TTAAATTATTCATATTCATATTTACCTTTATCATGGATATCTGGATTAA
TGATATTCTTAATATCTATTGTTACAGCTTTTATGGGTTATGTATTACC
TTGGGGTCAAATGAGTTTCTGGGGTGCTACTGTTATAACTAATTTACTT
TATTTTATTCCTGGACTTGTTTCATGGATTTGTGGTGGATATCTTGTAA
GTGACCCAACTTTAAAAAGATTCTTTGTATTACATTTTACTTTTCCATT
TATAGCTTTATGTATCGTATTTATACACATATTCTTCTTACATCTACAA
GGTAGCACTAATCCTTTAGGGTATGATACAGCTTTAAAAATACCCTTCT
ATCCAAATCTTTTAAGTCTTGACATTAAAGGATTTAATAATGTATTAGT
ATTATTCTTAGCTCAAAGTTTATTTGGAATATTACCATTATCACATCCA
GATAATGCAATTACAGTAGATAGATATGCTACACCTTTACATATTGTTC
CAGAATGGTATTTCTTACCTTTTTATGCAATGTTAAAAACCATTCCTAA
CAAAACTGCTGGTTTATTAGTTATGTTAGCATCACTACAAATATTATTT
CTATTAGCAGAACAAAGAAATTTAACAACTCTTATCCAATTTAAATTTG
CTTTTGGTGCTAGAGAATATTCAGTTCCTACAATTTGGTTTATATGTTC
ATTCTATGCTTTATTATGGATTGGATGTCAATTACCACAAGATATTTAC
ATTTTATATGGTCGTTTATTTATTATATTATTCTTTTTTAGTGGTTTAT
TTACACTTGTTCAATCTAAAAGAACACATTATGATTACAGCTCCCAAGC
AAACATTTAATATTACAAGGCTGCGATAAGACGACATTTCTGAGCATTG
AGCGGAACAATACAGACCGTAAGGTTATAATTATGTTTT
Node id 2:
SEQ ID NO: 97
AGTAGATACAAGAGCATATTTTACTTCTACAACTATTTTAATATCTATT
CCAACTGGTACAAAAGTATTTAATTGGATATGTACATATATGGGTAGTA
ATTTTGGAATAACACATAGTTCATCTCTTCTAGCATTATTATTTATATG
TACATTTACTTTTGGTGGTACTACTGGAGTAATATTAGGTAATGCTGCT
ATTGACATAGCTTTACATGATACATATTATGTAATTGCTCATTTCCACT
TTGTTCTATCAATTGGAGCAATTATTGCATTATTTACAACAGTAAGTGC
ATTCCAAGAAAATTTCTTTGGTAAACATTTACGTGAAAACTCAATTATT
ATATTATGGTCAATGTTATTCTTCGTAGGTGTAGTTTTAACATTTTTAC
CTATGCATTTCCTTGGATTTAATGTAATGCCTAGACGTATTCCTGATTA
TCCAGACGCTTTAAATGGTTGGAATATGATTTGCTCTATCGGATCAACT
ATGACTTTATTTGGTTTATTAATTTTTAAATAATATATAACTATTTTTT
GTTTATATGAATTATTATTCTATTAATTTAGCTAAAGCACACTTAATTC
ATTATCCATGTCCACTAAATATTAATTTCTTATGGAATTATGGATTTCT
TTTAGGAATAGTATTTTTTATACAAATTTTAACAGGTGTATTATTAGCA
ACTTGTTATACTCCAGAAATATCTTATGCATATTATAGTGTACAACACA
TATTAAGAGAATTATGGAGTGGATGGTGTTTTAGATATATGCATGCAAC
AGGTGCTTCATTTGTATTTATTTTAACTTACTTACATATTTTAAGAGGA
TTAAATTATTCATATTCATATTTACCTTTATCATGGATATCTGGA
Node id 3:
SEQ ID NO: 98
AGTAGATACAAGAGCATATTTTACTTCTACAACTATTTTAATATCTATT
CCAACTGGTACAAAAGTATTTAATTGGATATGTACATATATGGGTAGTA
ATTTTGGAATAACACATAGTTCATCTCTTCTAGCATTATTATTTATATG
TACATTTACTTTTGGTGGTACTACTGGAGTAATATTAGGTAATGCTGCT
ATTGACATAGCTTTACATGATACATATTATGTAATTGCTCATTTCCACT
TTGTTCTATCAATTGGAGCAATTATTGCATTATTTACAACAGTAAGTGC
ATTCCAAGAAAATTTCTTTGGTAAACATTTACGTGAAAACTCAATTATT
ATATTATGGTCAATGTTATTCTTCGTAGGTGTAGTTTTAACATTTTTAC
CTATGCATTTCCTTGGATTTAATGTAATGCCTAGACGTATTCCTGATT
01: AGTAGATACAAGAGCATATTTTACTTC
Node id 4:
SEQ ID NO: 99
AGTAGATACAAGAGCATATTTTACTTCTACAACTATTTTAATATCTATT
CCAACTGGTACAAAAGTATTTAATTGGATATGTACATATATGGGTAGTA
ATTTTGGAATAACACATAGTTCATCTCTTCTAGCATTATTATTTATATG
TACATTTACTTTTGGTGGTACTACTGGAGTAATATTAGGTAATGCTGCT
ATTGACATAGCTTTACATGATACAT
Node id 7:
SEQ ID NO: 100
ATTATGTAATTGCTCATTTCCACTTTGTTCTATCAATTGGAGCAATTAT
TGCATTATTTACAACAGTAAGTGCATTCCAAGAAAATTTCTTTGGTAAA
CATTTACGTGAAAACTCAATTATTATATTATGGTCAATGTTATTCTTCG
TAGGTGTAGTTTTAACATTTTTACCTATGCATTTCCTTGGATTTAATGT
AATGCCTAGACGTATTCCTGATT
Node id 10:
SEQ ID NO: 101
ATCCAGACGCTTTAAATGGTTGGAATATGATTTGCTCTATCGGATCAAC
TATGACTTTATTTGGTTTATTAATTTTTAAATAATATATAACTATTTTT
TGTTTATATGAATTATTATTCTATTAATTTAGCTAAAGCACACTTAATT
CATTATCCATGTCCACTAAATATTAATTTCTTATGGAATTATGGATTTC
TTTTAGGAATAGTATTTTTTATACAAATTTTAACAGGTGTATTATTAGC
AACTTGTTATACTCCAGAAATATCTTATGCATATTATAGTGTACAACAC
ATATTAAGAGAATTATGGAGTGGATGGTGTTTTAGATATATGCATGCAA
CAGGTGCTTCATTTGTATTTATTTTAACTTACTTACATATTTTAAGAGG
ATTAAATTATTCATATTCATATTTACCTTTATCATGGATATCTGGA
Node id 11:
SEQ ID NO: 102
ATCCAGACGCTTTAAATGGTTGGAATATGATTTGCTCTATCGGATCAAC
TATGACTTTATTTGGTTTATTAATTTTTAAATAATATATAACTATTTTT
TGTTTATATGAATTATTATTCTATTAATTTAGCTAAAGCACACTTAATT
CATTATCCATGTCCACTAAATATTAATTTCTTATGGAATTATGGATTTC
TTTTAGGAATAGTATTTTTTATACA
Node id 14:
SEQ ID NO: 103
AATTTTAACAGGTGTATTATTAGCAACTTGTTATACTCCAGAAATATCT
TATGCATATTATAGTGTACAACACATATTAAGAGAATTATGGAGTGGAT
GGTGTTTTAGATATATGCATGCAACAGGTGCTTCATTTGTATTTATTTT
AACTTACTTACATATTTTAAGAGGATTAAATTATTCATATTCATATTTA
CCTTTATCATGGATATCTGGA
Node id 17:
SEQ ID NO: 104
TTAATGATATTCTTAATATCTATTGTTACAGCTTTTATGGGTTATGTAT
TACCTTGGGGTCAAATGAGTTTCTGGGGTGCTACTGTTATAACTAATTT
ACTTTATTTTATTCCTGGACTTGTTTCATGGATTTGTGGTGGATATCTT
GTAAGTGACCCAACTTTAAAAAGATTCTTTGTATTACATTTTACTTTTC
CATTTATAGCTTTATGTATCGTATTTATACACATATTCTTCTTACATCT
ACAAGGTAGCACTAATCCTTTAGGGTATGATACAGCTTTAAAAATACCC
TTCTATCCAAATCTTTTAAGTCTTGACATTAAAGGATTTAATAATGTAT
TAGTATTATTCTTAGCTCAAAGTTTATTTGGAATATTACCATTATCACA
TCCAGATAATGCAATTACAGTAGATAGATATGCTACACCTTTACATATT
GTTCCAGAATGGTATTTCTTACCTTTTTATGCAATGTTAAAAACCATTC
CTAACAAAACTGCTGGTTTATTAGTTATGTTAGCATCACTACAAATATT
ATTTCTATTAGCAGAACAAAGAAATTTAACAACTCTTATCCAATTTAAA
TTTGCTTTTGGTGCTAGAGAATATTCAGTTCCTACAATTTGGTTTATAT
GTTCATTCTATGCTTTATTATGGATTGGATGTCAATTACCACAAGATAT
TTACATTTTATATGGTCGTTTATTTATTATATTATTCTTTTTTAGTGGT
TTATTTACACTTGTTCAATCTAAAAGAACACATTATGATTACAGCTCCC
AAGCAAACATTTAATATTACAAGGCTGCGATAAGACGACATTTCTGAGC
ATTGAGCGGAACAATACAGACCGTAAGGTTATAATTATGTTTT
Node id 18:
SEQ ID NO: 105
TTAATGATATTCTTAATATCTATTGTTACAGCTTTTATGGGTTATGTAT
TACCTTGGGGTCAAATGAGTTTCTGGGGTGCTACTGTTATAACTAATTT
ACTTTATTTTATTCCTGGACTTGTTTCATGGATTTGTGGTGGATATCTT
GTAAGTGACCCAACTTTAAAAAGATTCTTTGTATTACATTTTACTTTTC
CATTTATAGCTTTATGTATCGTATTTATACACATATTCTTCTTACATCT
ACAAGGTAGCACTAATCCTTTAGGGTATGATACAGCTTTAAAAATACCC
TTCTATCCAAATCTTTTAAGTCTTGACATTAAAGGATTTAATAATGTAT
TAGTATTATTCTTAGCTCAAAGTTTATTTGGAATATTACCATTATCACA
TCCAGATAATGCAATTACAGTAGATAGATATGCTACACCTTTACATA
Node id 19 LEN 220 Type 4:
SEQ ID NO: 106
TTAATGATATTCTTAATATCTATTGTTACAGCTTTTATGGGTTATGTAT
TACCTTGGGGTCAAATGAGTTTCTGGGGTGCTACTGTTATAACTAATTT
ACTTTATTTTATTCCTGGACTTGTTTCATGGATTTGTGGTGGATATCTT
GTAAGTGACCCAACTTTAAAAAGATTCTTTGTATTACATTTTACTTTTC
CATTTATAGCTTTATGTATCGTAT
Node id 22 LEN 219 Type 4:
SEQ ID NO: 107
TTATACACATATTCTTCTTACATCTACAAGGTAGCACTAATCCTTTAGG
GTATGATACAGCTTTAAAAATACCCTTCTATCCAAATCTTTTAAGTCTT
GACATTAAAGGATTTAATAATGTATTAGTATTATTCTTAGCTCAAAGTT
TATTTGGAATATTACCATTATCACATCCAGATAATGCAATTACAGTAGA
TAGATATGCTACACCTTTACATA
Node id 25:
SEQ ID NO: 108
TTGTTCCAGAATGGTATTTCTTACCTTTTTATGCAATGTTAAAAACCAT
TCCTAACAAAACTGCTGGTTTATTAGTTATGTTAGCATCACTACAAATA
TTATTTCTATTAGCAGAACAAAGAAATTTAACAACTCTTATCCAATTTA
AATTTGCTTTTGGTGCTAGAGAATATTCAGTTCCTACAATTTGGTTTAT
ATGTTCATTCTATGCTTTATTATGGATTGGATGTCAATTACCACAAGAT
ATTTACATTTTATATGGTCGTTTATTTATTATATTATTCTTTTTTAGTG
GTTTATTTACACTTGTTCAATCTAAAAGAACACATTATGATTACAGCTC
CCAAGCAAACATTTAATATTACAAGGCTGCGATAAGACGACATTTCTGA
GCATTGAGCGGAACAATACAGACCGTAAGGTTATAATTATGTTTT
Node id 26:
SEQ ID NO: 109
TTGTTCCAGAATGGTATTTCTTACCTTTTTATGCAATGTTAAAAACCAT
TCCTAACAAAACTGCTGGTTTATTAGTTATGTTAGCATCACTACAAATA
TTATTTCTATTAGCAGAACAAAGAAATTTAACAACTCTTATCCAATTTA
AATTTGCTTTTGGTGCTAGAGAATATTCAGTTCCTACAATTTGGTTTAT
ATGTTCATTCTATGCTTTATTAT
Node id 29:
SEQ ID NO: 110
GGATTGGATGTCAATTACCACAAGATATTTACATTTTATATGGTCGTTT
ATTTATTATATTATTCTTTTTTAGTGGTTTATTTACACTTGTTCAATCT
AAAAGAACACATTATGATTACAGCTCCCAAGCAAACATTTAATATTACA
AGGCTGCGATAAGACGACATTTCTGAGCATTGAGCGGAACAATACAGAC
CGTAAGGTTATAATTATGTTTT
CA Primer
SEQ ID NO: 111
CAACACACCACCCACCCAAC
>M1_Primer_1_smPCR_Adaptor
SEQ ID NO: 112
CAACACACCACCCACCCAACAGTAGATACAAGAGCATATTTTACTTC
>M1_Primer_2_smPCR_Adaptor
SEQ ID NO: 113
CAACACACCACCCACCCAACAAAACATAATTATAACCTTACGGTCTG
>M1_Primer_3_smPCR_Adaptor
SEQ ID NO: 114
CAACACACCACCCACCCAACATAGATATTAAGAATATCATTAATCCAGA
TATCCATGATAAAGGTAAAT
>M1_Primer_4_smPCR_Adaptor
SEQ ID NO: 115
CAACACACCACCCACCCAACACCTTTATCATGGATATCTGGATTAATGA
TATTCTTAATATCTATTGTTACAGC

1.-10. (canceled)

11. A method for cloning a target polynucleotide in the absence of a cell, comprising: analyzing the target polynucleotide to determine a plurality of shorter fragments; providing said plurality of shorter fragments as actual molecules; Amplifying said actual molecules as single stranded polynucleotides in a smPCR process; and constructing the target polynucleotide from said amplified actual molecules.

12. The method of claim 11, wherein said providing said plurality of shorter fragments further comprises amplifying said actual molecules according to a PCR process for introducing one or more sites for said smPCR process.

13. The method of claim 12, wherein said amplifying said actual molecules as single stranded polynucleotides in a smPCR process and said constructing the target polynucleotide from said amplified actual molecules comprise: Synthesizing a plurality of oligonucleotides;
Assembling said oligonucleotides to form a plurality of polynucleotide fragments;
Amplifying said polynucleotide fragments as single stranded polynucleotides in said smPCR process;
Assembling said fragments to form the target molecule.

14. The method of claim 13, wherein said polynucleotide fragments are up to about 500 bases in length.

15. The method of claim 14, wherein said assembling said fragments to form the target molecule further comprises:
Sequencing said fragments; and Selecting error-free fragments for said assembling.

16. The method of claim 15, wherein said analyzing the target polynucleotide further comprises determining a hierarchical process for preparing successively larger fragments at each level until the target molecule is constructed; and wherein said assembling said oligonucleotides to form a plurality of polynucleotide fragments and said assembling said fragments to form the target molecule are performed according to said hierarchical process.

17. The method of claim 16, wherein said hierarchical process is determined by performing the Divide and Conquer analytical method.

18. The method of claim 13, wherein said synthesizing said plurality of oligonucleotides comprises synthesizing at least one oligonucleotide featuring an error.

19. The method of claim 15, performed automatically without manual intervention.

20. (canceled)


SEQUENCE LISTING (115 sequences)

SEQ ID NO: 1 - DNA, 768 bp, Artificial Sequence
  GFP fragments
ggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagc

SEQ ID NO: 2 - DNA, 440 bp, Artificial Sequence
  GFP fragments
ggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcaca

SEQ ID NO: 3 - DNA, 358 bp, Artificial Sequence
  GFP fragments
tcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagc

SEQ ID NO: 4 - DNA, 242 bp, Artificial Sequence
  GFP fragments
ggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgct

SEQ ID NO: 5 - DNA, 222 bp, Artificial Sequence
  GFP fragments
acggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcaca

SEQ ID NO: 6 - DNA, 180 bp, Artificial Sequence
  GFP fragments
tcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcg

SEQ ID NO: 7 - DNA, 202 bp, Artificial Sequence
  GFP fragments
accagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagc

SEQ ID NO: 8 - DNA, 133 bp, Artificial Sequence
  GFP fragments
ggatccaccggtcgccaccatggtgagcaagggcgaggagctgttcaccggggtggtgcccatcctggtcgagctggacggcgacgtaaacggccacaagttcagcgtgtccggcgagggcgagggcgatgcc

SEQ ID NO: 9 - DNA, 134 bp, Artificial Sequence
  GFP fragments
gtccggcgagggcgagggcgatgccacctacggcaagctgaccctgaagttcatctgcaccaccggcaagctgcccgtgccctggcccaccctcgtgaccaccctgacctacggcgtgcagtgcttcagccgct

SEQ ID NO: 10 - DNA, 135 bp, Artificial Sequence
  GFP fragments
acggcgtgcagtgcttcagccgctaccccgaccacatgaagcagcacgacttcttcaagtccgccatgcccgaaggctacgtccaggagcgcaccatcttcttcaaggacgacggcaactacaagacccgcgccg

SEQ ID NO: 11 - DNA, 119 bp, Artificial Sequence
  GFP fragments
caaggacgacggcaactacaagacccgcgccgaggtgaagttcgagggcgacaccctggtgaaccgcatcgagctgaagggcatcgacttcaaggaggacggcaacatcctggggcaca

SEQ ID NO: 12 - DNA, 95 bp, Artificial Sequence
  GFP fragments
tcaaggaggacggcaacatcctggggcacaagctggagtacaactacaacagccacaacgtctatatcatggccgacaagcagaagaacggcatc

SEQ ID NO: 13 - DNA, 110 bp, Artificial Sequence
  GFP fragments
ggccgacaagcagaagaacggcatcaaggtgaacttcaagatccgccacaacatcgaggacggcagcgtgcagctcgccgaccactaccagcagaacacccccatcggcg

SEQ ID NO: 14 - DNA, 102 bp, Artificial Sequence
  GFP fragments
accagcagaacacccccatcggcgacggccccgtgctgctgcccgacaaccactacctgagcacccagtccgccctgagcaaagaccccaacgagaagcgcg

SEQ ID NO: 15 - DNA, 126 bp, Artificial Sequence
  GFP fragments
gagcaaagaccccaacgagaagcgcgatcacatggtcctgctggagttcgtgaccgccgccgggatcactctcggcatggacgagctgtacaagtaaagcggccgcgactctagatcataatcagc

SEQ ID NO: 16 - DNA, 77 bp, Artificial Sequence
  GFP Oligo
ggatccaccggtcgccaccatggtgagcaagggcgaggagctgttcaccggggtggtgcccatcctggtcgagctgg

SEQ ID NO: 17 - DNA, 79 bp, Artificial Sequence
  GFP Oligo
ggcatcgccctcgccctcgccggacacgctgaacttgtggccgtttacgtcgccgtccagctcgaccaggatgggcacc

SEQ ID NO: 18 - DNA, 68 bp, Artificial Sequence
  GFP Oligo
gtccggcgagggcgagggcgatgccacctacggcaagctgaccctgaagttcatctgcaccaccggca

SEQ ID NO: 19 - DNA, 84 bp, Artificial Sequence
  GFP Oligo
agcggctgaagcactgcacgccgtaggtcagggtggtcacgagggtgggccagggcacgggcagcttgccggtggtgcagatga

SEQ ID NO: 20 - DNA, 84 bp, Artificial Sequence
  GFP Oligo
acggcgtgcagtgcttcagccgctaccccgaccacatgaagcagcacgacttcttcaagtccgccatgcccgaaggctacgtcc

SEQ ID NO: 21 - DNA, 80 bp, Artificial Sequence
  GFP Oligo
cggcgcgggtcttgtagttgccgtcgtccttgaagaagatggtgcgctcctggacgtagccttcgggcatggcggacttg

SEQ ID NO: 22 - DNA, 67 bp, Artificial Sequence
  GFP Oligo
caaggacgacggcaactacaagacccgcgccgaggtgaagttcgagggcgacaccctggtgaaccgc

SEQ ID NO: 23 - DNA, 83 bp, Artificial Sequence
  GFP Oligo
tgtgccccaggatgttgccgtcctccttgaagtcgatgcccttcagctcgatgcggttcaccagggtgtcgccctcgaacttc

SEQ ID NO: 24 - DNA, 63 bp, Artificial Sequence
  GFP Oligo
tcaaggaggacggcaacatcctggggcacaagctggagtacaactacaacagccacaacgtct

SEQ ID NO: 25 - DNA, 61 bp, Artificial Sequence
  GFP Oligo
gatgccgttcttctgcttgtcggccatgatatagacgttgtggctgttgtagttgtactcc

SEQ ID NO: 26 - DNA, 64 bp, Artificial Sequence
  GFP Oligo
ggccgacaagcagaagaacggcatcaaggtgaacttcaagatccgccacaacatcgaggacggc

SEQ ID NO: 27 - DNA, 74 bp, Artificial Sequence
  GFP Oligo
cgccgatgggggtgttctgctggtagtggtcggcgagctgcacgctgccgtcctcgatgttgtggcggatcttg

SEQ ID NO: 28 - DNA, 62 bp, Artificial Sequence
  GFP Oligo
accagcagaacacccccatcggcgacggccccgtgctgctgcccgacaaccactacctgagc

SEQ ID NO: 29 - DNA, 62 bp, Artificial Sequence
  GFP Oligo
cgcgcttctcgttggggtctttgctcagggcggactgggtgctcaggtagtggttgtcgggc

SEQ ID NO: 30 - DNA, 80 bp, Artificial Sequence
  GFP Oligo
gagcaaagaccccaacgagaagcgcgatcacatggtcctgctggagttcgtgaccgccgccgggatcactctcggcatgg

SEQ ID NO: 31 - DNA, 75 bp, Artificial Sequence
  GFP Oligo
gctgattatgatctagagtcgcggccgctttacttgtacagctcgtccatgccgagagtgatcccggcggcggtc

SEQ ID NO: 32 - DNA, 17 bp, Artificial Sequence
  GFP Primer
ggatccaccggtcgcca

SEQ ID NO: 33 - DNA, 15 bp, Artificial Sequence
  GFP Primer
gtccggcgagggcga

SEQ ID NO: 34 - DNA, 17 bp, Artificial Sequence
  GFP Primer
acggcgtgcagtgcttc

SEQ ID NO: 35 - DNA, 21 bp, Artificial Sequence
  GFP Primer
caaggacgacggcaactacaa

SEQ ID NO: 36 - DNA, 19 bp, Artificial Sequence
  GFP Primer
tcaaggaggacggcaacat

SEQ ID NO: 37 - DNA, 19 bp, Artificial Sequence
  GFP Primer
ggccgacaagcagaagaac

SEQ ID NO: 38 - DNA, 19 bp, Artificial Sequence
  GFP Primer
accagcagaacacccccat

SEQ ID NO: 39 - DNA, 20 bp, Artificial Sequence
  GFP Primer
gagcaaagaccccaacgaga

SEQ ID NO: 40 - DNA, 15 bp, Artificial Sequence
  GFP Primer
ggcatcgccctcgcc

SEQ ID NO: 41 - DNA, 19 bp, Artificial Sequence
  GFP Primer
agcggctgaagcactgcac

SEQ ID NO: 42 - DNA, 16 bp, Artificial Sequence
  GFP Primer
cggcgcgggtcttgta

SEQ ID NO: 43 - DNA, 17 bp, Artificial Sequence
  GFP Primer
tgtgccccaggatgttg

SEQ ID NO: 44 - DNA, 21 bp, Artificial Sequence
  GFP Primer
gatgccgttcttctgcttgtc

SEQ ID NO: 45 - DNA, 15 bp, Artificial Sequence
  GFP Primer
cgccgatgggggtgt

SEQ ID NO: 46 - DNA, 16 bp, Artificial Sequence
  primer
cgcgcttctcgttggg

SEQ ID NO: 47 - DNA, 25 bp, Artificial Sequence
  GFP Primer
ggctgattatgatctagagtcgcgg

SEQ ID NO: 48 - DNA, 27 bp, Artificial Sequence
  1.8Kb Mitochondrial DNA fragment primer
agtagatacaagagcatattttacttc

SEQ ID NO: 49 - DNA, 27 bp, Artificial Sequence
  1.8Kb Mitochondrial DNA fragment primer
aaaacataattataaccttacggtctg

SEQ ID NO: 50 - DNA, 49 bp, Artificial Sequence
  1.8Kb Mitochondrial DNA fragment primer
atagatattaagaatatcattaatccagatatccatgataaaggtaaat

SEQ ID NO: 51 - DNA, 54 bp, Artificial Sequence
  1.8Kb Mitochondrial DNA fragment primer
acctttatcatggatatctggattaatgatattcttaatatctattgttacagc

SEQ ID NO: 52 - DNA, 31 bp, Artificial Sequence
  1.8Kb Mitochondrial DNA fragment primer
gcgtctggataatcaggaatacgtctaggca

SEQ ID NO: 53 - DNA, 40 bp, Artificial Sequence
  1.8Kb Mitochondrial DNA fragment primer
ctagacgtattcctgattatccagacgctttaaatggttg

SEQ ID NO: 54 - DNA, 41 bp, Artificial Sequence
  1.8Kb Mitochondrial DNA fragment primer
aattacataatatgtatcatgtaaagctatgtcaatagcag

SEQ ID NO: 55 - DNA, 56 bp, Artificial Sequence
  1.8Kb Mitochondrial DNA fragment primer
ctgctattgacatagctttacatgatacatattatgtaattgctcatttccacttt

SEQ ID NO: 56 - DNA, 68 bp, Artificial Sequence
  1.8Kb Mitochondrial DNA fragment primer
agtagatacaagagcatattttacttctacaactattttaatatctattccaactggtacaaaagtat

SEQ ID NO: 57 - DNA, 70 bp, Artificial Sequence
  1.8Kb Mitochondrial DNA fragment primer
atgtatcatgtaaagctatgtcaatagcagcattacctaatattactccagtagtaccaccaaaagtaaa

SEQ ID NO: 58 - DNA, 66 bp, Artificial Sequence
  1.8Kb Mitochondrial DNA fragment primer
attatgtaattgctcatttccactttgttctatcaattggagcaattattgcattatttacaacag

SEQ ID NO: 59 - DNA, 68 bp, Artificial Sequence
  1.8Kb Mitochondrial DNA fragment primer
aatcaggaatacgtctaggcattacattaaatccaaggaaatgcataggtaaaaatgttaaaactaca

SEQ ID NO: 60 - DNA, 59 bp, Artificial Sequence
  1.8Kb Mitochondrial DNA fragment primer
gttgctaataatacacctgttaaaatttgtataaaaaatactattcctaaaagaaatcc

SEQ ID NO: 61 - DNA, 49 bp, Artificial Sequence
  1.8Kb Mitochondrial DNA fragment primer
aggaatagtattttttatacaaattttaacaggtgtattattagcaact

SEQ ID NO: 62 - DNA, 78 bp, Artificial Sequence
  1.8Kb Mitochondrial DNA fragment primer
atccagacgctttaaatggttggaatatgatttgctctatcggatcaactatgactttatttggtttattaattttta

SEQ ID NO: 63 - DNA, 74 bp, Artificial Sequence
  1.8Kb Mitochondrial DNA fragment primer
tgtataaaaaatactattcctaaaagaaatccataattccataagaaattaatatttagtggacatggataatg

SEQ ID NO: 64 - DNA, 71 bp, Artificial Sequence
  1.8Kb Mitochondrial DNA fragment primer
aattttaacaggtgtattattagcaacttgttatactccagaaatatcttatgcatattatagtgtacaac

SEQ ID NO: 65 - DNA, 72 bp, Artificial Sequence
  primer
tccagatatccatgataaaggtaaatatgaatatgaataatttaatcctcttaaaatatgtaagtaagttaa

SEQ ID NO: 66 - DNA, 58 bp, Artificial Sequence
  1.8Kb Mitochondrial DNA fragment primer
aagaaataccattctggaacaatatgtaaaggtgtagcatatctatctactgtaattg

SEQ ID NO: 67 - DNA, 42 bp, Artificial Sequence
  1.8Kb Mitochondrial DNA fragment primer
gctacacctttacatattgttccagaatggtatttcttacct

SEQ ID NO: 68 - DNA, 37 bp, Artificial Sequence
  1.8Kb Mitochondrial DNA fragment primer
atgtgtataaatacgatacataaagctataaatggaa

SEQ ID NO: 69 - DNA, 67 bp, Artificial Sequence
  1.8Kb Mitochondrial DNA fragment primer
cattttacttttccatttatagctttatgtatcgtatttatacacatattcttcttacatctacaag

SEQ ID NO: 70 - DNA, 67 bp, Artificial Sequence
  1.8Kb Mitochondrial DNA fragment primer
ttaatgatattcttaatatctattgttacagcttttatgggttatgtattaccttggggtcaaatga

SEQ ID NO: 71 - DNA, 69 bp, Artificial Sequence
  1.8Kb Mitochondrial DNA fragment primer
atacgatacataaagctataaatggaaaagtaaaatgtaatacaaagaatctttttaaagttgggtcac

SEQ ID NO: 72 - DNA, 69 bp, Artificial Sequence
  1.8Kb Mitochondrial DNA fragment primer
ttatacacatattcttcttacatctacaaggtagcactaatcctttagggtatgatacagctttaaaaa

SEQ ID NO: 73 - DNA, 68 bp, Artificial Sequence
  1.8Kb Mitochondrial DNA fragment primer
tatgtaaaggtgtagcatatctatctactgtaattgcattatctggatgtgataatggtaatattcca

SEQ ID NO: 74 - DNA, 47 bp, Artificial Sequence
  1.8Kb Mitochondrial DNA fragment primer
catccaatccataataaagcatagaatgaacatataaaccaaattgt

SEQ ID NO: 75 - DNA, 57 bp, Artificial Sequence
  1.8Kb Mitochondrial DNA fragment primer
ttggtttatatgttcattctatgctttattatggattggatgtcaattaccacaaga

SEQ ID NO: 76 - DNA, 66 bp, Artificial Sequence
  1.8Kb Mitochondrial DNA fragment primer
ttgttccagaatggtatttcttacctttttatgcaatgttaaaaaccattcctaacaaaactgctg

SEQ ID NO: 77 - DNA, 63 bp, Artificial Sequence
  1.8Kb Mitochondrial DNA fragment primer
ataataaagcatagaatgaacatataaaccaaattgtaggaactgaatattctctagcaccaa

SEQ ID NO: 78 - DNA, 75 bp, Artificial Sequence
  1.8Kb Mitochondrial DNA fragment primer
ggattggatgtcaattaccacaagatatttacattttatatggtcgtttatttattatattattcttttttagtg

SEQ ID NO: 79 - DNA, 61 bp, Artificial Sequence
  1.8Kb Mitochondrial DNA fragment primer
aaaacataattataaccttacggtctgtattgttccgctcaatgctcagaaatgtcgtctt

SEQ ID NO: 80 - DNA, 79 bp, Artificial Sequence
  1.8Kb Mitochondrial DNA fragment oligo
tatctattccaactggtacaaaagtatttaattggatatgtacatatatgggtagtaattttggaataacacatagttc

SEQ ID NO: 81 - DNA, 80 bp, Artificial Sequence
  1.8Kb Mitochondrial DNA fragment oligo
attactccagtagtaccaccaaaagtaaatgtacatataaataataatgctagaagagatgaactatgtgttattccaaa

SEQ ID NO: 82 - DNA, 80 bp, Artificial Sequence
  1.8Kb Mitochondrial DNA fragment oligo
agcaattattgcattatttacaacagtaagtgcattccaagaaaatttctttggtaaacatttacgtgaaaactcaatta

SEQ ID NO: 83 - DNA, 78 bp, Artificial Sequence
  1.8Kb Mitochondrial DNA fragment oligo
atgcataggtaaaaatgttaaaactacacctacgaagaataacattgaccataatataataattgagttttcacgtaa

SEQ ID NO: 84 - DNA, 80 bp, Artificial Sequence
  1.8Kb Mitochondrial DNA fragment oligo
atcaactatgactttatttggtttattaatttttaaataatatataactattttttgtttatatgaattattattctatt

SEQ ID NO: 85 - DNA, 80 bp, Artificial Sequence
  1.8Kb Mitochondrial DNA fragment oligo
agaaattaatatttagtggacatggataatgaattaagtgtgctttagctaaattaatagaataataattcatataaaca

SEQ ID NO: 86 - DNA, 80 bp, Artificial Sequence
  1.8Kb Mitochondrial DNA fragment oligo
agaaatatcttatgcatattatagtgtacaacacatattaagagaattatggagtggatggtgttttagatatatgcatg

SEQ ID NO: 87 - DNA, 79 bp, Artificial Sequence
  1.8Kb Mitochondrial DNA fragment oligo
atttaatcctcttaaaatatgtaagtaagttaaaataaatacaaatgaagcacctgttgcatgcatatatctaaaacac

SEQ ID NO: 88 - DNA, 78 bp, Artificial Sequence
  1.8Kb Mitochondrial DNA fragment oligo
ttatgtattaccttggggtcaaatgagtttctggggtgctactgttataactaatttactttattttattcctggact

SEQ ID NO: 89 - DNA, 80 bp, Artificial Sequence
  1.8Kb Mitochondrial DNA fragment oligo
acaaagaatctttttaaagttgggtcacttacaagatatccaccacaaatccatgaaacaagtccaggaataaaataaag

SEQ ID NO: 90 - DNA, 80 bp, Artificial Sequence
  1.8Kb Mitochondrial DNA fragment oligo
ctttagggtatgatacagctttaaaaatacccttctatccaaatcttttaagtcttgacattaaaggatttaataatgta

SEQ ID NO: 91 - DNA, 79 bp, Artificial Sequence
  1.8Kb Mitochondrial DNA fragment oligo
ctggatgtgataatggtaatattccaaataaactttgagctaagaataatactaatacattattaaatcctttaatgtc

SEQ ID NO: 92 - DNA, 78 bp, Artificial Sequence
  1.8Kb Mitochondrial DNA fragment oligo
aaaaaccattcctaacaaaactgctggtttattagttatgttagcatcactacaaatattatttctattagcagaaca

SEQ ID NO: 93 - DNA, 80 bp, Artificial Sequence
  1.8Kb Mitochondrial DNA fragment oligo
aactgaatattctctagcaccaaaagcaaatttaaattggataagagttgttaaatttctttgttctgctaatagaaata

SEQ ID NO: 94 - DNA, 80 bp, Artificial Sequence
  1.8Kb Mitochondrial DNA fragment oligo
ggtcgtttatttattatattattcttttttagtggtttatttacacttgttcaatctaaaagaacacattatgattacag

SEQ ID NO: 95 - DNA, 76 bp, Artificial Sequence
  1.8Kb Mitochondrial DNA fragment oligo
atgctcagaaatgtcgtcttatcgcagccttgtaatattaaatgtttgcttgggagctgtaatcataatgtgttct

SEQ ID NO: 96 - DNA, 1754 bp, Artificial Sequence
  1.8Kb Mitochondrial DNA fragment node
agtagatacaagagcatattttacttctacaactattttaatatctattccaactggtacaaaagtatttaattggatatgtacatatatgggtagtaattttggaataacacatagttcatctcttctagcattattatttatatgtacatttacttttggtggtactactggagtaatattaggtaatgctgctattgacatagctttacatgatacatattatgtaattgctcatttccactttgttctatcaattggagcaattattgcattatttacaacagtaagtgcattccaagaaaatttctttggtaaacatttacgtgaaaactcaattattatattatggtcaatgttattcttcgtaggtgtagttttaacatttttacctatgcatttccttggatttaatgtaatgcctagacgtattcctgattatccagacgctttaaatggttggaatatgatttgctctatcggatcaactatgactttatttggtttattaatttttaaataatatataactattttttgtttatatgaattattattctattaatttagctaaagcacacttaattcattatccatgtccactaaatattaatttcttatggaattatggatttcttttaggaatagtattttttatacaaattttaacaggtgtattattagcaacttgttatactccagaaatatcttatgcatattatagtgtacaacacatattaagagaattatggagtggatggtgttttagatatatgcatgcaacaggtgcttcatttgtatttattttaacttacttacatattttaagaggattaaattattcatattcatatttacctttatcatggatatctggattaatgatattcttaatatctattgttacagcttttatgggttatgtattaccttggggtcaaatgagtttctggggtgctactgttataactaatttactttattttattcctggacttgtttcatggatttgtggtggatatcttgtaagtgacccaactttaaaaagattctttgtattacattttacttttccatttatagctttatgtatcgtatttatacacatattcttcttacatctacaaggtagcactaatcctttagggtatgatacagctttaaaaatacccttctatccaaatcttttaagtcttgacattaaaggatttaataatgtattagtattattcttagctcaaagtttatttggaatattaccattatcacatccagataatgcaattacagtagatagatatgctacacctttacatattgttccagaatggtatttcttacctttttatgcaatgttaaaaaccattcctaacaaaactgctggtttattagttatgttagcatcactacaaatattatttctattagcagaacaaagaaatttaacaactcttatccaatttaaatttgcttttggtgctagagaatattcagttcctacaatttggtttatatgttcattctatgctttattatggattggatgtcaattaccacaagatatttacattttatatggtcgtttatttattatattattcttttttagtggtttatttacacttgttcaatctaaaagaacacattatgattacagctcccaagcaaacatttaatattacaaggctgcgataagacgacatttctgagcattgagcggaacaatacagaccgtaaggttataattatgtttt

SEQ ID NO: 97 - DNA, 878 bp, Artificial Sequence
  1.8Kb Mitochondrial DNA fragment node
agtagatacaagagcatattttacttctacaactattttaatatctattccaactggtacaaaagtatttaattggatatgtacatatatgggtagtaattttggaataacacatagttcatctcttctagcattattatttatatgtacatttacttttggtggtactactggagtaatattaggtaatgctgctattgacatagctttacatgatacatattatgtaattgctcatttccactttgttctatcaattggagcaattattgcattatttacaacagtaagtgcattccaagaaaatttctttggtaaacatttacgtgaaaactcaattattatattatggtcaatgttattcttcgtaggtgtagttttaacatttttacctatgcatttccttggatttaatgtaatgcctagacgtattcctgattatccagacgctttaaatggttggaatatgatttgctctatcggatcaactatgactttatttggtttattaatttttaaataatatataactattttttgtttatatgaattattattctattaatttagctaaagcacacttaattcattatccatgtccactaaatattaatttcttatggaattatggatttcttttaggaatagtattttttatacaaattttaacaggtgtattattagcaacttgttatactccagaaatatcttatgcatattatagtgtacaacacatattaagagaattatggagtggatggtgttttagatatatgcatgcaacaggtgcttcatttgtatttattttaacttacttacatattttaagaggattaaattattcatattcatatttacctttatcatggatatctgga

SEQ ID NO: 98 - DNA, 467 bp, Artificial Sequence
  1.8Kb Mitochondrial DNA fragment node
agtagatacaagagcatattttacttctacaactattttaatatctattccaactggtacaaaagtatttaattggatatgtacatatatgggtagtaattttggaataacacatagttcatctcttctagcattattatttatatgtacatttacttttggtggtactactggagtaatattaggtaatgctgctattgacatagctttacatgatacatattatgtaattgctcatttccactttgttctatcaattggagcaattattgcattatttacaacagtaagtgcattccaagaaaatttctttggtaaacatttacgtgaaaactcaattattatattatggtcaatgttattcttcgtaggtgtagttttaacatttttacctatgcatttccttggatttaatgtaatgcctagacgtattcctgattagtagatacaagagcatattttacttc

SEQ ID NO: 99 - DNA, 221 bp, Artificial Sequence
  1.8Kb Mitochondrial DNA fragment node
agtagatacaagagcatattttacttctacaactattttaatatctattccaactggtacaaaagtatttaattggatatgtacatatatgggtagtaattttggaataacacatagttcatctcttctagcattattatttatatgtacatttacttttggtggtactactggagtaatattaggtaatgctgctattgacatagctttacatgatacat

SEQ ID NO: 100 - DNA, 219 bp, Artificial Sequence
  1.8Kb Mitochondrial DNA fragment node
attatgtaattgctcatttccactttgttctatcaattggagcaattattgcattatttacaacagtaagtgcattccaagaaaatttctttggtaaacatttacgtgaaaactcaattattatattatggtcaatgttattcttcgtaggtgtagttttaacatttttacctatgcatttccttggatttaatgtaatgcctagacgtattcctgatt

SEQ ID NO: 101 - DNA, 438 bp, Artificial Sequence
  1.8Kb Mitochondrial DNA fragment node
atccagacgctttaaatggttggaatatgatttgctctatcggatcaactatgactttatttggtttattaatttttaaataatatataactattttttgtttatatgaattattattctattaatttagctaaagcacacttaattcattatccatgtccactaaatattaatttcttatggaattatggatttcttttaggaatagtattttttatacaaattttaacaggtgtattattagcaacttgttatactccagaaatatcttatgcatattatagtgtacaacacatattaagagaattatggagtggatggtgttttagatatatgcatgcaacaggtgcttcatttgtatttattttaacttacttacatattttaagaggattaaattattcatattcatatttacctttatcatggatatctgga

SEQ ID NO: 102 - DNA, 221 bp, Artificial Sequence
  1.8Kb Mitochondrial DNA fragment node
atccagacgctttaaatggttggaatatgatttgctctatcggatcaactatgactttatttggtttattaatttttaaataatatataactattttttgtttatatgaattattattctattaatttagctaaagcacacttaattcattatccatgtccactaaatattaatttcttatggaattatggatttcttttaggaatagtattttttataca

SEQ ID NO: 103 - DNA, 217 bp, Artificial Sequence
  1.8Kb Mitochondrial DNA fragment node
aattttaacaggtgtattattagcaacttgttatactccagaaatatcttatgcatattatagtgtacaacacatattaagagaattatggagtggatggtgttttagatatatgcatgcaacaggtgcttcatttgtatttattttaacttacttacatattttaagaggattaaattattcatattcatatttacctttatcatggatatctgga

SEQ ID NO: 104 - DNA, 876 bp, Artificial Sequence
  1.8Kb Mitochondrial DNA fragment node
ttaatgatattcttaatatctattgttacagcttttatgggttatgtattaccttggggtcaaatgagtttctggggtgctactgttataactaatttactttattttattcctggacttgtttcatggatttgtggtggatatcttgtaagtgacccaactttaaaaagattctttgtattacattttacttttccatttatagctttatgtatcgtatttatacacatattcttcttacatctacaaggtagcactaatcctttagggtatgatacagctttaaaaatacccttctatccaaatcttttaagtcttgacattaaaggatttaataatgtattagtattattcttagctcaaagtttatttggaatattaccattatcacatccagataatgcaattacagtagatagatatgctacacctttacatattgttccagaatggtatttcttacctttttatgcaatgttaaaaaccattcctaacaaaactgctggtttattagttatgttagcatcactacaaatattatttctattagcagaacaaagaaatttaacaactcttatccaatttaaatttgcttttggtgctagagaatattcagttcctacaatttggtttatatgttcattctatgctttattatggattggatgtcaattaccacaagatatttacattttatatggtcgtttatttattatattattcttttttagtggtttatttacacttgttcaatctaaaagaacacattatgattacagctcccaagcaaacatttaatattacaaggctgcgataagacgacatttctgagcattgagcggaacaatacagaccgtaaggttataattatgtttt

SEQ ID NO: 105 - DNA, 439 bp, Artificial Sequence
  1.8Kb Mitochondrial DNA fragment node
ttaatgatattcttaatatctattgttacagcttttatgggttatgtattaccttggggtcaaatgagtttctggggtgctactgttataactaatttactttattttattcctggacttgtttcatggatttgtggtggatatcttgtaagtgacccaactttaaaaagattctttgtattacattttacttttccatttatagctttatgtatcgtatttatacacatattcttcttacatctacaaggtagcactaatcctttagggtatgatacagctttaaaaatacccttctatccaaatcttttaagtcttgacattaaaggatttaataatgtattagtattattcttagctcaaagtttatttggaatattaccattatcacatccagataatgcaattacagtagatagatatgctacacctttacata

SEQ ID NO: 106 - DNA, 220 bp, Artificial Sequence
  1.8Kb Mitochondrial DNA fragment node
ttaatgatattcttaatatctattgttacagcttttatgggttatgtattaccttggggtcaaatgagtttctggggtgctactgttataactaatttactttattttattcctggacttgtttcatggatttgtggtggatatcttgtaagtgacccaactttaaaaagattctttgtattacattttacttttccatttatagctttatgtatcgtat

SEQ ID NO: 107 - DNA, 219 bp, Artificial Sequence
  1.8Kb Mitochondrial DNA fragment node
ttatacacatattcttcttacatctacaaggtagcactaatcctttagggtatgatacagctttaaaaatacccttctatccaaatcttttaagtcttgacattaaaggatttaataatgtattagtattattcttagctcaaagtttatttggaatattaccattatcacatccagataatgcaattacagtagatagatatgctacacctttacata

SEQ ID NO: 108 - DNA, 437 bp, Artificial Sequence
  1.8Kb Mitochondrial DNA fragment node
ttgttccagaatggtatttcttacctttttatgcaatgttaaaaaccattcctaacaaaactgctggtttattagttatgttagcatcactacaaatattatttctattagcagaacaaagaaatttaacaactcttatccaatttaaatttgcttttggtgctagagaatattcagttcctacaatttggtttatatgttcattctatgctttattatggattggatgtcaattaccacaagatatttacattttatatggtcgtttatttattatattattcttttttagtggtttatttacacttgttcaatctaaaagaacacattatgattacagctcccaagcaaacatttaatattacaaggctgcgataagacgacatttctgagcattgagcggaacaatacagaccgtaaggttataattatgtttt

SEQ ID NO: 109 - DNA, 219 bp, Artificial Sequence
  1.8Kb Mitochondrial DNA fragment node
ttgttccagaatggtatttcttacctttttatgcaatgttaaaaaccattcctaacaaaactgctggtttattagttatgttagcatcactacaaatattatttctattagcagaacaaagaaatttaacaactcttatccaatttaaatttgcttttggtgctagagaatattcagttcctacaatttggtttatatgttcattctatgctttattat

SEQ ID NO: 110 - DNA, 218 bp, Artificial Sequence
  1.8Kb Mitochondrial DNA fragment node
ggattggatgtcaattaccacaagatatttacattttatatggtcgtttatttattatattattcttttttagtggtttatttacacttgttcaatctaaaagaacacattatgattacagctcccaagcaaacatttaatattacaaggctgcgataagacgacatttctgagcattgagcggaacaatacagaccgtaaggttataattatgtttt

SEQ ID NO: 111 - DNA, 20 bp, Artificial Sequence
  primer
caacacaccacccacccaac

SEQ ID NO: 112 - DNA, 47 bp, Artificial Sequence
  primer
caacacaccacccacccaacagtagatacaagagcatattttacttc

SEQ ID NO: 113 - DNA, 47 bp, Artificial Sequence
  primer
caacacaccacccacccaacaaaacataattataaccttacggtctg

SEQ ID NO: 114 - DNA, 69 bp, Artificial Sequence
  primer
caacacaccacccacccaacatagatattaagaatatcattaatccagatatccatgataaaggtaaat

SEQ ID NO: 115 - DNA, 74 bp, Artificial Sequence
  primer
caacacaccacccacccaacacctttatcatggatatctggattaatgatattcttaatatctattgttacagc